(19) United States
(12) Patent Application Publication
(10) Pub. No.: US 2023/0192719 A1
(43) Pub. Date: Jun. 22, 2023

(54) ORGANIC COMPOUND AND ORGANIC LIGHT-EMITTING ELEMENT

(71) Applicant: CANON KABUSHIKI KAISHA, Tokyo (JP)

(72) Inventors: HIROKAZU MIYASHITA, Kanagawa (JP); JUN KAMATANI, Tokyo (JP); NAOKI YAMADA, Tokyo (JP); HIRONOBU IWAWAKI, Kanagawa (JP); HIROKI OHRUI, Tokyo (JP); YOSUKE NISHIDE, Kanagawa (JP)

(21) Appl. No.: 18/065,868

(22) Filed: Dec. 14, 2022

(30) Foreign Application Priority Data
Dec. 17, 2021 (JP) 2021-205221

(51) Int. Cl. C07D 498/06; H10K 85/60; C07D 513/06; C07D 519/00; C07D 471/06; C07D 498/16; C07D 513/16; C07D 471/16; C07F 7/08; H10K 85/40; H10K 85/30; H10K 50/80

(52) U.S. Cl. CPC C07D 498/06; H01L 51/0071; H01L 51/0073; C07D 513/06; C07D 519/00; C07D 471/06; H01L 51/0072; H01L 51/0067; H01L 51/0074; C07D 498/16; C07D 513/16; C07D 471/16; C07F 7/0812; H01L 51/0094; H01L 51/0064; H01L 51/007; H01L 51/0085; H01L 51/52; C07B 2200/05

(57) ABSTRACT

An organic compound represented by the formula [1]:

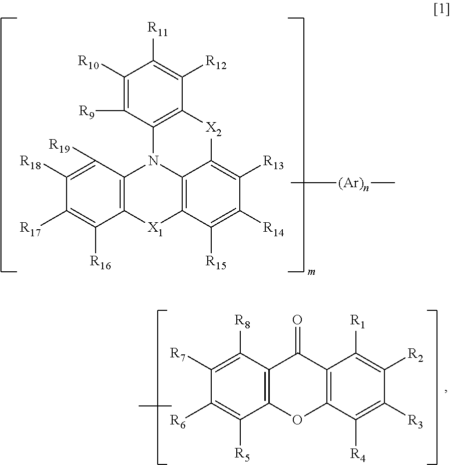

R₁ to R₁₉ are independently selected from the group consisting of a hydrogen atom, a deuterium atom, a halogen atom, and a substituent, and R₉ and R₁₉ are optionally bonded together. When R₉ and R₁₉ are bonded together, the bonding is selected from the group consisting of direct bonding, bonding via an oxygen atom or a sulfur atom, and bonding via CR₂₀R₂₁. R₂₀ and R₂₁ independently have the same meaning as R₁ to R₁₉. X₁ and X₂ independently denote an oxygen atom, a sulfur atom, CR₂₀R₂₁, or a single bond. Ar denotes an aromatic hydrocarbon group or a heterocyclic group, n denotes an integer in the range of 0 to 2, and m denotes an integer in the range of 1 to 4. When n is 0, any one of R₁ to R₈ is directly bonded to any one of R₉ to R₁₉.

### BACKGROUND OF THE INVENTION

### Field of the Invention

The present disclosure relates to an organic compound and an organic light-emitting element including the organic compound.

### Description of the Related Art

An organic light-emitting element (hereinafter sometimes referred to as an “organic electroluminescent element” or an “organic EL element”) is an electronic element that includes a pair of electrodes and an organic compound layer between the electrodes. Electrons and holes are injected from the pair of electrodes to generate an exciton of a light-emitting organic compound in the organic compound layer. When the exciton returns to its ground state, the organic light-emitting element emits light.

With recent significant advances in organic light-emitting elements, it is possible to realize low drive voltage, various emission wavelengths, high-speed responsivity, and thin and light light-emitting devices.

Compounds suitable for organic light-emitting elements have been actively developed. This is because a compound that provides an element with good emission properties and lifetime characteristics is important for high-performance organic light-emitting elements. As a compound developed, WO 2006/114966 describes the following compound 1-A as an example of a compound with a xanthone moiety. WO 2015/002213 describes the following compound 1-B.

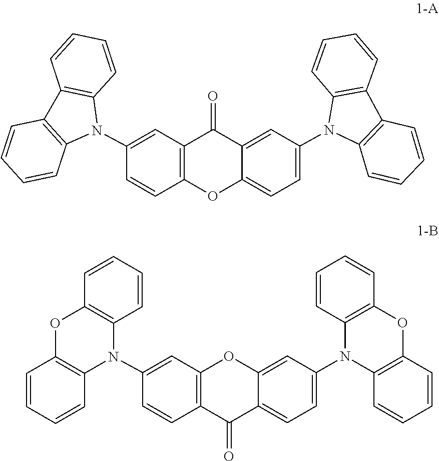 [SMILES: O=c1c2cc(-n3c4ccccc4c4ccccc43)ccc2oc2ccc(-n3c4ccccc4c4ccccc43)cc12 ; O=c1c2ccc(N3c4ccccc4Oc4ccccc43)cc2oc2cc(N3c4ccccc4Oc4ccccc43)ccc12]

The present inventors have found that the compounds 1-A and 1-B have low triplet energy (T1 energy), as described later, and organic light-emitting elements produced by using the compounds 1-A and 1-B therefore have low light emission efficiency and have room for improvement in durability.

### SUMMARY OF THE INVENTION

The present disclosure provides an organic compound that provides an organic light-emitting element with good emission properties and durability, and provides an organic light-emitting element using the organic compound.

An organic compound according to the present disclosure is represented by the following formula [1]:

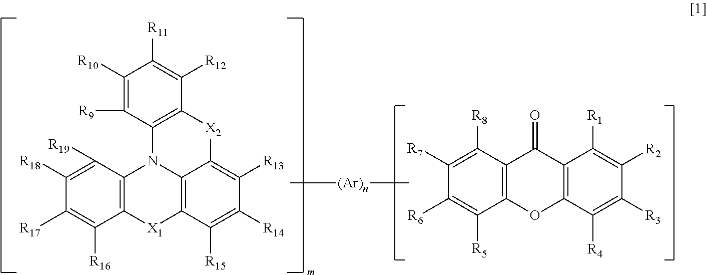

wherein R₁ to R₁₉ are independently selected from the group consisting of a hydrogen atom, a deuterium atom, a halogen atom, a substituted or unsubstituted alkyl group, a substituted or unsubstituted alkoxy group, a substituted or unsubstituted amino group, a substituted or unsubstituted aryloxy group, a substituted or unsubstituted heteroaryloxy group, a substituted or unsubstituted silyl group, a substituted or unsubstituted aromatic hydrocarbon group, a substituted or unsubstituted heterocyclic group, and a cyano group, and R₉ and R₁₉ are optionally bonded together,

when R₉ and R₁₉ are bonded together, the bonding is selected from the group consisting of direct bonding, bonding via an oxygen atom, bonding via a sulfur atom, and bonding via CR₂₀R₂₁, wherein R₂₀ and R₂₁ independently have the same meaning as R₁ to R₁₉,

X₁ and X₂ independently denote an oxygen atom, a sulfur atom, CR₂₀R₂₁, or a single bond,

Ar denotes a substituted or unsubstituted aromatic hydrocarbon group or a substituted or unsubstituted heterocyclic group,

n denotes an integer in the range of 0 to 2, and when n is 0, any one of R₁ to R₈ is directly bonded to any one of R₉ to R₁₉, and

m denotes an integer in the range of 1 to 4.

Further features of the present disclosure will become apparent from the following description of exemplary embodiments with reference to the attached drawings.

### BRIEF DESCRIPTION OF THE DRAWINGS

FIG. 1 is a table comparing the flatness of an electron-donating moiety between an organic compound according to the present disclosure and a comparative compound.

FIG. 2 is a table comparing the distributions of HOMO and LUMO between an organic compound according to the present disclosure and a comparative compound.

FIG. 3 is a table comparing the flatness of an electron-donating moiety between organic compounds according to the present disclosure.

FIG. 4A is a schematic cross-sectional view of an example of a pixel of a display apparatus according to an embodiment of the present disclosure.

FIG. 4B is a schematic cross-sectional view of an example of a display apparatus including an organic light-emitting element according to an embodiment of the present disclosure.

FIG. 5A is a schematic view of an image-forming apparatus according to an embodiment of the present disclosure.

FIG. 5B is a schematic view of a plurality of light-emitting portions of an exposure light source arranged on a long substrate.

FIG. 5C is a schematic view of a plurality of light-emitting portions of an exposure light source arranged on a long substrate.

FIG. 6 is a schematic view of an example of a display apparatus according to an embodiment of the present disclosure.

FIG. 7A is a schematic view of an example of an imaging apparatus according to an embodiment of the present disclosure.

FIG. 7B is a schematic view of an example of electronic equipment according to an embodiment of the present disclosure.

FIG. 8A is a schematic view of an example of a display apparatus according to an embodiment of the present disclosure.

FIG. 8B is a schematic view of an example of a foldable display apparatus.

FIG. 9A is a schematic view of an example of a lighting apparatus according to an embodiment of the present disclosure.

FIG. 9B is a schematic view of an example of an automobile with a vehicle lamp according to an embodiment of the present disclosure.

FIG. 10A is a schematic view of an example of a wearable device according to an embodiment of the present disclosure.

FIG. 10B is a schematic view of an example of a wearable device according to an embodiment of the present disclosure with an imaging apparatus.

### DESCRIPTION OF THE EMBODIMENTS

The present disclosure discloses an organic compound represented by the following general formula [1]:

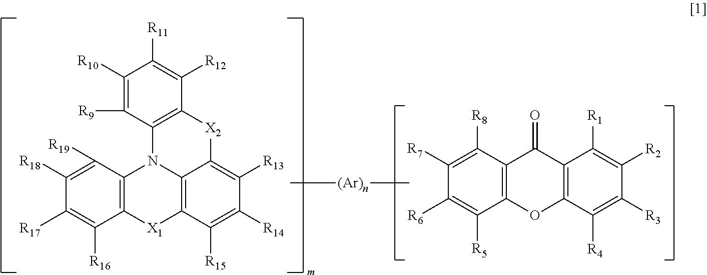

wherein R₁ to R₁₉ are independently selected from the group consisting of a hydrogen atom, a deuterium atom, a halogen atom, a substituted or unsubstituted alkyl group, a substituted or unsubstituted alkoxy group, a substituted or unsubstituted amino group, a substituted or unsubstituted aryloxy group, a substituted or unsubstituted heteroaryloxy group, a substituted or unsubstituted silyl group, a substituted or unsubstituted aromatic hydrocarbon group, a substituted or unsubstituted heterocyclic group, and a cyano group, and R₉ and R₁₉ are bonded together, when R₉ and R₁₉ are bonded together, the bonding is selected from the group consisting of direct bonding, bonding via an oxygen atom, bonding via a sulfur atom, and bonding via CR₂₀R₂₁, wherein R₂₀ and R₂₁ independently have the same meaning as R₁ to R₁₉,

X₁ and X₂ independently denote an oxygen atom, a sulfur atom, CR₂₀R₂₁, or a single bond,

Ar denotes a substituted or unsubstituted aromatic hydrocarbon group or a substituted or unsubstituted heterocyclic group,

n denotes an integer in the range of 0 to 2, and when n is 0, any one of R₁ to R₈ is directly bonded to any one of R₉ to R₁₉, and

m denotes an integer in the range of 1 to 4.

The halogen atom, alkyl group, alkoxy group, amino group, aryloxy group, heteroaryloxy group, silyl group, aromatic hydrocarbon group, and heterocyclic group denoted by R₁ to R₂₁ are more specifically described.

Examples of the halogen atom include, but are not limited to, fluorine, chlorine, bromine, and iodine.

Examples of the alkyl group include, but are not limited to, a methyl group, an ethyl group, a n-propyl group, an isopropyl group, a n-butyl group, a t-butyl group, a sec-butyl group, an octyl group, a cyclohexyl group, a 1-adamantyl group, and a 2-adamantyl group.

Examples of the alkoxy group include, but are not limited to, a methoxy group, an ethoxy group, a propoxy group, a 2-ethyl-octyloxy group, and a benzyloxy group.

Examples of the amino group include, but are not limited to, an N-methylamino group, an N-ethylamino group, an N,N-dimethylamino group, an N,N-diethylamino group, an N-methyl-N-ethylamino group, an N-benzylamino group, an N-methyl-N-benzylamino group, an N,N-dibenzylamino group, an anilino group, an N,N-diphenylamino group, an N,N-dinaphthylamino group, an N,N-difluorenylamino group, an N-phenyl-N-tolylamino group, an N,N-ditolylamino group, an N-methyl-N-phenylamino group, an N,N-dianisolylamino group, an N-mesityl-N-phenylamino group, an N,N-dimesitylamino group, an N-phenyl-N-(4-t-butylphenyl)amino group, an N-phenyl-N-(4-trifluoromethylphenyl)amino group, and an N-piperidyl group.

Examples of the aryloxy group and the heteroaryloxy group include, but are not limited to, a phenoxy group and a thienyloxy group.

Examples of the silyl group include, but are not limited to, a trimethylsilyl group and a triphenylsilyl group.

Examples of the aromatic hydrocarbon group, but are not limited to, a phenyl group, a naphthyl group, an indenyl group, a biphenyl group, a terphenyl group, a fluorenyl group, a phenanthryl group, a fluoranthenyl group, and a triphenylenyl group.

Examples of the heterocyclic group include, but are not limited to, a pyridyl group, an oxazolyl group, an oxadiazolyl group, a thiazolyl group, a thiadiazolyl group, a carbazolyl group, an acridinyl group, a phenanthrolyl group, a dibenzofuranyl group, and a dibenzothiophenyl group.

An additional optional substituent of the alkyl group, alkoxy group, amino group, aryloxy group, heteroaryloxy group, silyl group, aromatic hydrocarbon group, and heterocyclic group may be, but is not limited to, a halogen atom, such as fluorine, chlorine, bromine, or iodine; an alkyl group, such as a methyl group, an ethyl group, a n-propyl group, an isopropyl group, a n-butyl group, or a t-butyl group; an alkoxy group, such as a methoxy group, an ethoxy group, or a propoxy group; an amino group, such as a dimethylamino group, a diethylamino group, a dibenzylamino group, a diphenylamino group, or a ditolylamino group; an aryloxy group, such as a phenoxy group; an aromatic hydrocarbon group, such as a phenyl group or a biphenyl group; a heterocyclic group, such as a pyridyl group or a pyrrolyl group; a cyano group, or a deuterium atom.

Next, a method for synthesizing the organic compound according to the present embodiment is described. For example, the organic compound according to the present embodiment is synthesized in accordance with the following reaction scheme.

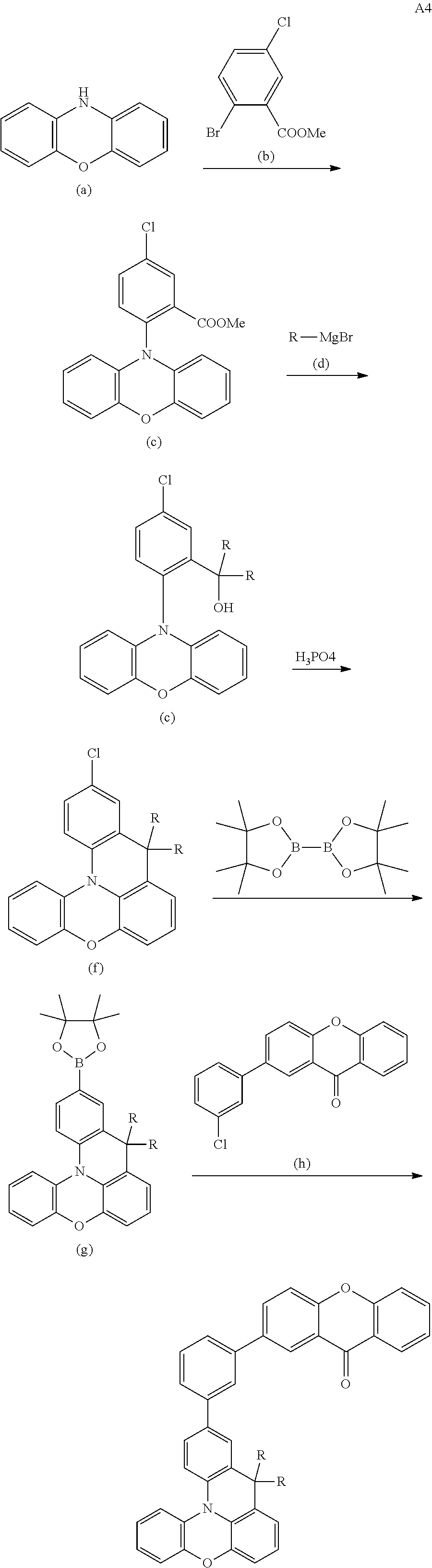

The compounds represented by (a) to (h) can be appropriately chosen to produce various compounds. The present embodiment is not limited to the synthesis scheme described above, and various synthesis schemes and reagents can be used. The synthesis method is described in detail in exemplary embodiments.

Next, properties of the organic compound according to the present embodiment are described. The organic compound according to the present embodiment has the following characteristics (1) to (4) and therefore has high triplet energy (T1 energy) and stability. Furthermore, the organic compound can be used to provide an organic light-emitting element with high light emission efficiency and durability.

(1) The electron-donating moiety has a structure in which triphenylamine is cross-linked, and is not bonded to a xanthone skeleton, which is an electron-withdrawing moiety, via the N atom. Thus, the T1 energy is high.
(2) The electron-donating moiety has a structure in which phenyl groups of triphenylamine are cross-linked at two or more positions, and therefore has high bond stability.
(3) The electron-donating moiety has a structure in which triphenylamine is cross-linked, and therefore has low flatness.
(4) The highest occupied molecular orbital (HOMO) and the lowest unoccupied molecular orbital (LUMO) are separated in the electron-donating moiety and the electron-withdrawing moiety. Thus, the organic compound is useful as a bipolar host.

The characteristics (1) to (4) are described below.

(1) The electron-donating moiety has a structure in which triphenylamine is cross-linked, and is not bonded to a xanthone skeleton, which is an electron-withdrawing moiety, via the N atom. Thus, the T1 energy is high.

In the disclosure of the organic compound according to the present embodiment, the present inventors have focused on the structure of an electron-donating moiety and an electron-withdrawing moiety. More specifically, the organic compound according to the present embodiment has a structure in which triphenylamine is cross-linked as an electron-donating moiety, and has a xanthone skeleton as an electron-withdrawing moiety. Thus, the triplet energy (T1) is high. This is because an electron-donating moiety and an electron-withdrawing moiety in the molecule induce an excited state due to charge transfer (CT) transition and result in high T1 energy.

Table 1 shows the result of comparing triplet energy (T1) between an exemplary compound A4, which is the organic compound according to the present embodiment, and comparative compounds 1 and 2. The comparative compound 1 is the compound 1-A described in Patent Literature 1. The comparative compound 2 has a structure similar to the compound 1-B described in Patent Literature 2. The compound 1-B has phenoxazine bonded to positions 3 and 6 of xanthone, whereas the comparative compound 2 has phenoxazine bonded to positions 2 and 7 of xanthone. The compound 1-B and the comparative compound 2 are considered to have almost the same T1 energy. The T1 energy was measured with “F-4500” manufactured by Hitachi, Ltd. by photoluminescence (PL) in a diluted toluene container at 77 k at an excitation wavelength of 350 nm and was calculated from a peak wavelength on the short-wavelength side of an emission spectrum.

TABLE 1

| | Structure | T1 (nm) |
|---|---|---|
| Exemplary compound A4 | 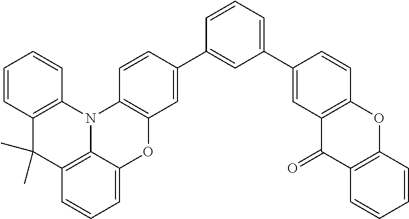 [SMILES: CC1(C)c2ccccc2N2c3ccc(-c4cccc(-c5ccc6oc7ccccc7c(=O)c6c5)c4)cc3Oc3cccc1c32] | 457 |
| Comparative compound 1 | 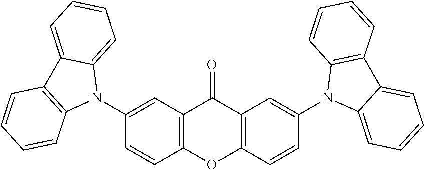 [SMILES: O=c1c2cc(-n3c4ccccc4c4ccccc43)ccc2oc2ccc(-n3c4ccccc4c4ccccc43)cc12] | 475 |
| Comparative compound 2 | 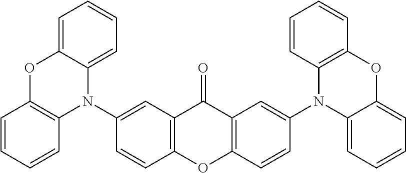 [SMILES: O=c1c2cc(N3c4ccccc4Oc4ccccc43)ccc2oc2ccc(N3c4ccccc4Oc4ccccc43)cc12] | 511 |

Table 1 shows that the exemplary compound A4 had T1 of 457 nm. On the other hand, the comparative compound 1 had T1 of 475 nm, and the comparative compound 2 had T1 of 511 nm. Thus, the exemplary compound A4 has higher T1 energy. This was considered as follows.

In the comparative compounds 1 and 2, the electron-donating moiety bonded to the xanthone skeleton via the N atom enhances the CT between the electron-donating moiety and the electron-withdrawing moiety and lowers the T1 energy. In contrast, the organic compound according to the present embodiment represented by the general formula [1], which has a structure in which the electron-donating moiety is bonded to the xanthone skeleton via at least one phenyl group, can appropriately maintain the CT between the electron-donating moiety and the electron-withdrawing moiety and have high T1 energy.

The effects of the high T1 energy in the organic light-emitting element are described below. Phosphorescent elements are organic light-emitting elements that use the T1 energy for light emission. A light-emitting layer host material of an organic light-emitting element and a peripheral layer in contact with the light-emitting layer should have higher T1 energy than phosphorescent materials that emit phosphorescence. Thus, when the organic compound according to the present embodiment is used for a phosphorescent element, particularly as a host material for a light-emitting layer, due to its high T1 energy, the organic compound according to the present embodiment can provide efficient element characteristics.

Delayed fluorescent elements are organic light-emitting elements that use S1 energy for light emission by reverse intersystem crossing. To induce the reverse intersystem crossing, the T1 energy should be sufficiently high to decrease the difference between the S1 energy and the T1 energy. Thus, when the organic compound according to the present embodiment is used for a delayed fluorescent element, particularly used with a light-emitting material of a light-emitting layer, due to its high T1 energy, the organic compound according to the present embodiment is expected to provide efficient element characteristics.

(2) The electron-donating moiety has a structure in which triphenylamine is cross-linked, and therefore has high bond stability.

In the disclosure of the organic compound according to the present embodiment, the present inventors have focused on the bond stability of an electron-donating moiety. More specifically, in the organic compound according to the present embodiment, phenyl groups of triphenylamine are cross-linked. Thus, the organic compound has high bond stability.

Table 2 shows the result of comparing the bond distance between an exemplary compound B4, which is the organic compound according to the present embodiment, and the comparative compounds 1 and 2. The bond stability increases as the bond distance decreases. The bond indicated by “a” in Table 2 is the bond with the longest bond distance in the molecule.

TABLE 2

| | Structure | Bond distance |
|---|---|---|
| Exemplary compound B4 | 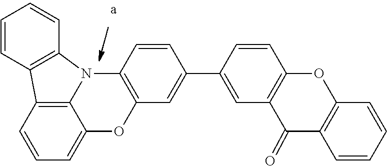 [SMILES: O=c1c2ccccc2oc2ccc(-c3ccc4c(c3)Oc3cccc5c6ccccc6n-4c35)cc12] | 1.41 |
| Comparative compound 1 | 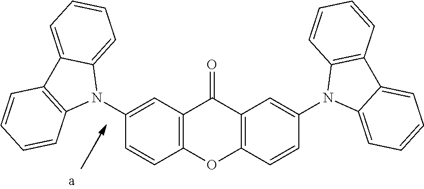 [SMILES: O=c1c2cc(-n3c4ccccc4c4ccccc43)ccc2oc2ccc(-n3c4ccccc4c4ccccc43)cc12] | 1.42 |
| Comparative compound 2 | 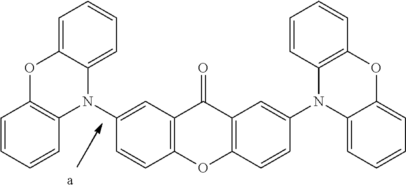 [SMILES: O=c1c2cc(N3c4ccccc4Oc4ccccc43)ccc2oc2ccc(N3c4ccccc4Oc4ccccc43)cc12] | 1.43 |

Table 2 shows that, in the comparative compounds 1 and 2, the C—N bond between the electron-donating moiety and the electron-withdrawing moiety has the longest bond distance. In contrast, in the organic compound according to the present embodiment, in which triphenylamine has a cross-linked structure, the C—N bond of the electron-donating moiety has a shorter bond distance. This results in higher bond stability.

Furthermore, the cross-linked structure has the following effects. In the comparative compounds 1 and 2, bond cleavage in the C—N bond with low bond stability converts the structure into a different structure by disproportionation, that is, decomposes the structure. In contrast, in the organic compound according to the present embodiment, even when bond cleavage occurs in the C—N bond, due to the cross-linked structure, recombination occurs with high probability and can reduce decomposition.

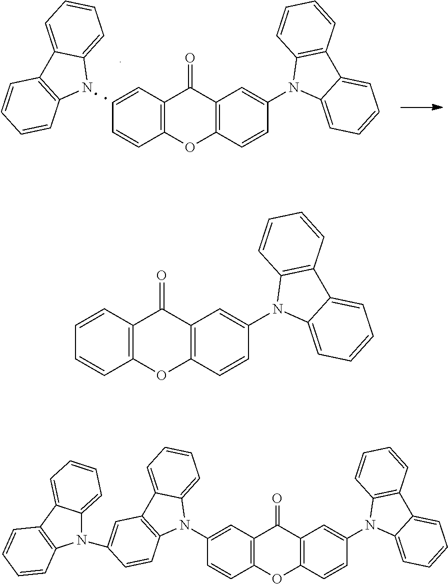

In the case of an organic compound according to the present disclosure

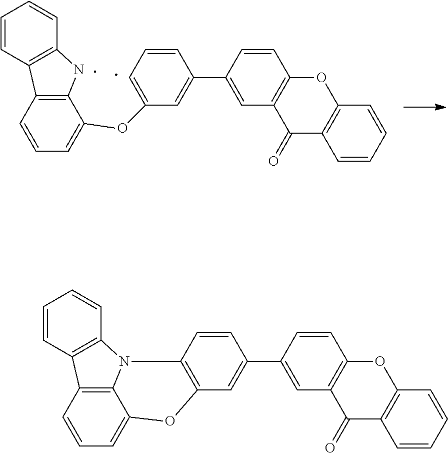

Thus, it is understood that the organic compound according to the present embodiment has a stable bond that forms the molecular structure. An organic light-emitting element including the organic compound according to the present embodiment has high durability because the molecule with high bond stability rarely decomposes.

The bond distance was estimated by molecular orbital calculation. The calculation method in the molecular orbital calculation method utilized a widely used density functional theory (DFT). B3LYP was used as the functional, and 6-31G* was used as the basis function. The molecular orbital calculation method was performed using widely used Gaussian 09 (Gaussian 09, Revision C. 01, M. J. Frisch, G. W. Trucks, H. B. Schlegel, G. E. Scuseria, M. A. Robb, J. R. Cheeseman, G. Scalmani, V. Barone, B. Mennucci, G. A. Petersson, H. Nakatsuji, M. Caricato, X. Li, H. P. Hratchian, A. F. Izmaylov, J. Bloino, G. Zheng, J. L. Sonnenberg, M. Hada, M. Ehara, K. Toyota, R. Fukuda, J. Hasegawa, M. Ishida, T. Nakajima, Y. Honda, O. Kitao, H. Nakai, T. Vreven, J. A. Montgomery, Jr., J. E. Peralta, F. Ogliaro, M. Bearpark, J. J. Heyd, E. Brothers, K. N. Kudin, V. N. Staroverov, T. Keith, R. Kobayashi, J. Normand, K. Raghavachari, A. Rendell, J. C. Burant, S. S. Iyengar, J. Tomasi, M. Cossi, N. Rega, J. M. Millam, M. Klene, J. E. Knox, J. B. Cross, V. Bakken, C. Adamo, J. Jaramillo, R. Gomperts, R. E. Stratmann, O. Yazyev, A. J. Austin, R. Cammi, C. Pomelli, J. W. Ochterski, R. L. Martin, K. Morokuma, V. G. Zakrzewski, G. A. Voth, P. Salvador, J. J. Dannenberg, S. Dapprich, A. D. Daniels, O. Farkas, J. B. Foresman, J. V. Ortiz, J. Cioslowski, and D. J. Fox, Gaussian, Inc., Wallingford Conn., 2010.). This method was used for the molecular orbital calculation described in the present specification.

(3) The electron-donating moiety has a structure in which triphenylamine is cross-linked, and therefore has low flatness.

In the disclosure of the organic compound according to the present embodiment, the present inventors have focused on the flatness of an electron-donating moiety. More specifically, in the organic compound according to the present embodiment, the xanthone skeleton, which is the electron-withdrawing moiety, has high flatness, and the electron-donating moiety can therefore have low flatness. This is because the electron-donating moiety and the electron-withdrawing moiety both having high flatness result in high crystallinity. In other words, this is not preferable because this promotes molecular packing in which molecules are superposed with each other and impairs film properties. The organic compound according to the present embodiment has the electron-donating moiety with low flatness, which suppresses molecular packing and crystallization and enhances amorphous properties.

Enhanced amorphous properties, that is, enhanced film properties are preferred for organic light-emitting elements. This is because high amorphous properties reduce the generation of crystal grain boundaries, trap levels, and quenchers associated with fine crystallization even while the element is driven, and high carrier transport ability and efficient emission properties can be maintained. Consequently, an organic light-emitting element with high durability and efficiency can be provided.

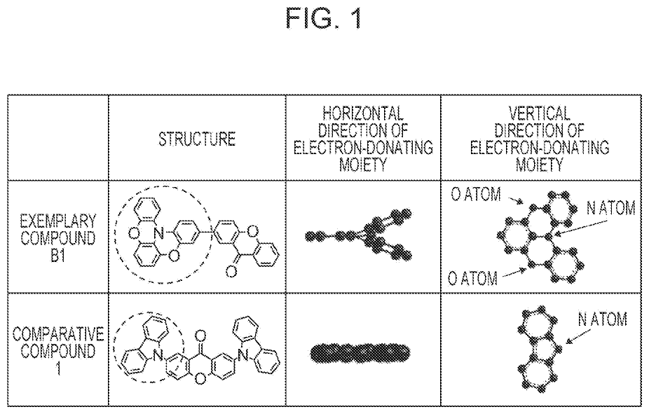

FIG. 1 shows the result of comparing the flatness of the electron-donating moiety (the circular dotted line portion in the table) between an exemplary compound B1, which is the organic compound according to the present embodiment, and the comparative compound 1. As illustrated in FIG. 1, the comparative compound 1 has a carbazole skeleton with high flatness in the electron-donating moiety. In contrast, the exemplary compound B1 has triphenylamine with a cross-linked structure in the electron-donating moiety and has lower flatness due to the steric repulsion of the phenyl groups.

Thus, the organic compound according to the present embodiment has good film properties and can provide an organic light-emitting element with high durability and efficiency.

(4) The HOMO and the LUMO are separated in the electron-donating moiety and the electron-withdrawing moiety. Thus, the organic compound is useful as a bipolar host.

In the disclosure of the organic compound according to the present embodiment, the present inventors have focused on charge separation between HOMO and LUMO. More specifically, in the organic compound according to the present embodiment, HOMO is localized in a cross-linked triphenylamine, which is the electron-donating moiety, and LUMO is localized in the xanthone skeleton, which is the electron-withdrawing moiety. Thus, the organic compound is useful as a bipolar host. This is because triphenylamine typically has electron-donating ability and is resistant to oxidation but is not resistant to reduction. In other words, it is vulnerable to electrons. Thus, the molecular orbital of LUMO may not be distributed to triphenylamine. On the other hand, the xanthone skeleton has electron-withdrawing ability and is resistant to reduction but is not resistant to oxidation. In other words, it is vulnerable to holes. Thus, the molecular orbital of HOMO may not be distributed to the xanthone skeleton. In short, HOMO can be localized in the electron-donating moiety, and LUMO can be localized in the electron-withdrawing moiety.

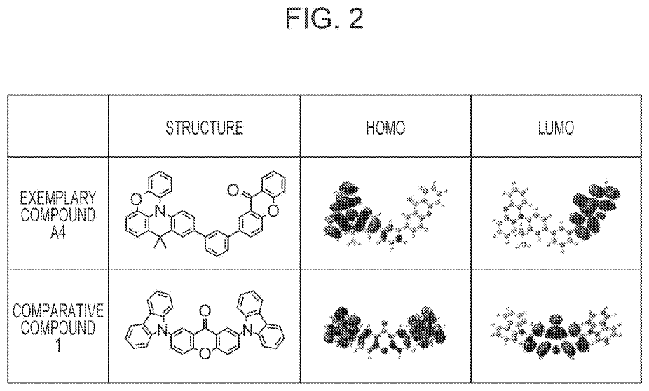

FIG. 2 shows the result of comparing the distributions of HOMO and LUMO between the organic compound according to the present embodiment and the comparative compound. In FIG. 2, in the organic compound according to the present embodiment, HOMO is localized in a cross-linked triphenylamine, which is the electron-donating moiety, and LUMO is localized in xanthone, which is the electron-withdrawing moiety. Thus, it is a bipolar host with separated functions. In contrast, the comparative compound 1 is not preferable as a bipolar host because HOMO is distributed to the xanthone moiety.

The evaluation of emission properties and lifetime characteristics of the organic compound according to the present embodiment described in the characteristics (1) to (4) is described in more detail in the exemplary embodiments described later.

When further having the following characteristics, the organic compound according to the present embodiment can be particularly suitably used for an organic light-emitting element.

(5) In the compound represented by the general formula [1], at least one of X₁ and X₂ is CR₂₀R₂₁.
(6) In the compound represented by the general formula [1], at least one of X₁ and X₂ contains an oxygen atom or a sulfur atom.
(7) In the general formula [1], m is 1, and the triphenylamine skeleton is bonded to any one of R₂, R₃, R₆, and R₇ of the xanthone skeleton.

These characteristics are described below.

(5) A compound in which at least one of X₁ and X₂ is CR₂₀R₂₁.

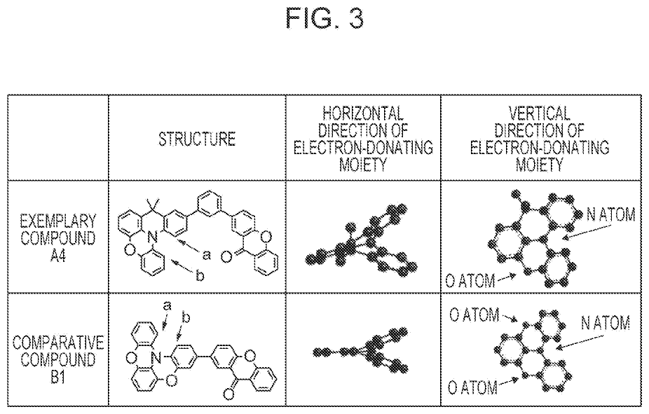

In the organic compound according to the present embodiment, at least one of X₁ and X₂ can be CR₂₀R₂₁. This is because such a structure has a substituent in the direction perpendicular to the in-plane direction of the electron-donating moiety and can therefore particularly suppress the overlap between electron-donating moieties. In FIG. 3, the flatness of the electron-donating moiety of the organic compound according to the present embodiment is compared. In FIG. 3, the flatness of the electron-donating moiety is compared in terms of the dihedral angle of benzene indicated by “a” and “b”.

The exemplary compound A4 had a dihedral angle of 41 degrees, whereas the exemplary compound B1 had a dihedral angle of 33 degrees. This shows that the flatness of a cross-linked triphenylamine is lower in the exemplary compound A4. Furthermore, due to CR₂₀R₂₁ (in the exemplary compound A4, R₂₀ and R₂₁ are methyl groups), there is a substituent in the direction perpendicular to the in-plane direction of the electron-donating moiety. This can more effectively suppress planar molecular packing.

(6) A compound in which at least one of X₁ and X₂ contains an oxygen atom or a sulfur atom.

The organic compound according to the present embodiment can contain an oxygen atom or a sulfur atom in the cross-linking moiety of triphenylamine. Abundant lone pairs in these atoms can enhance charge transport ability and make it particularly easy to adjust the carrier balance of the compound. Thus, the compound is useful as a bipolar host and can be used as a host material for a light-emitting layer.

(7) In the general formula [1], m is 1, and the triphenylamine skeleton is bonded to any one of R₂, R₃, R₆, and R₇ of the xanthone skeleton.

In the organic compound according to the present embodiment, m in the general formula [1] can be 1. This is because the organic compound is useful as a bipolar host when m is 1.

The distributions of HOMO and LUMO of the exemplary compounds A4 and A19 are compared as shown in Table 3. The exemplary compound A4 has m=1, whereas the exemplary compound A19 has m=2. In the exemplary compound A19, LUMO localized in the xanthone skeleton makes it difficult to transfer electrons to an adjacent molecule via the LUMO. This is because the LUMO localized in the xanthone skeleton is surrounded by an electron-donating moiety present at both ends, which inhibits carrier transfer between LUMOs. This slightly decreases electron-transport ability and leaves room for improvement as a bipolar host.

In contrast, in the exemplary compound A4, only one electron-donating moiety does not surround LUMO localized in the xanthone skeleton and does not inhibit electron transfer between LUMOs. Thus, the exemplary compound A4 can maintain good electron-transport ability and is useful as a bipolar host.

TABLE 3

| | Structure | HOMO | LUMO |
|---|---|---|---|
| Exemplary compound A4 | 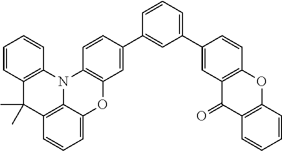 [SMILES: CC1(C)c2ccccc2N2c3ccc(-c4cccc(-c5ccc6oc7ccccc7c(=O)c6c5)c4)cc3Oc3cccc1c32] | 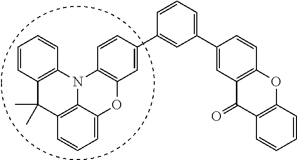 | 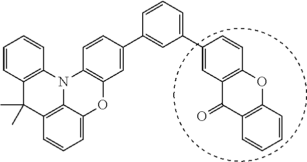 |
| Exemplary compound A19 | 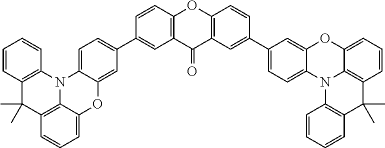 | 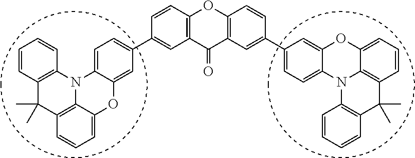 | 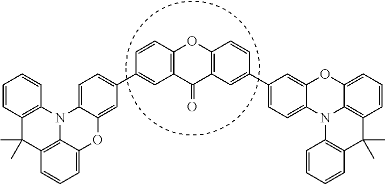 |

The triphenylamine skeleton can be bonded to any one of R₂, R₃, R₆, and R₇ of the xanthone skeleton. This is because bonding at these substitution positions decreases the bond distance. A shorter bond distance results in higher bond stability and a more stable compound.

Specific examples of the organic compound according to the present embodiment are described below. However, the present embodiments are not limited to these examples.

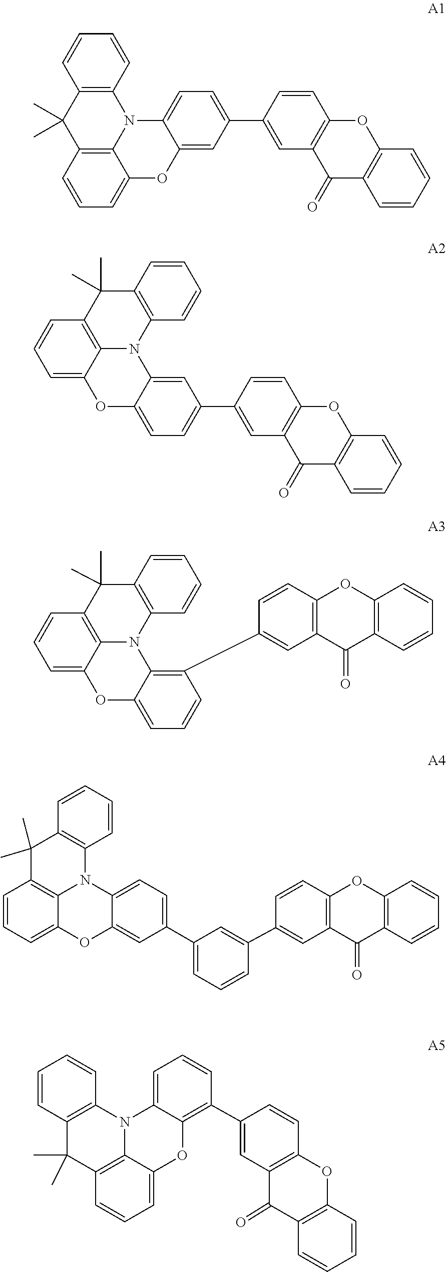 [SMILES: CC1(C)c2ccccc2N2c3ccc(-c4ccc5oc6ccccc6c(=O)c5c4)cc3Oc3cccc1c32 ; CC1(C)c2ccccc2N2c3cc(-c4ccc5oc6ccccc6c(=O)c5c4)ccc3Oc3cccc1c32 ; CC1(C)c2ccccc2N2c3c(cccc3-c3ccc4oc5ccccc5c(=O)c4c3)Oc3cccc1c32 ; CC1(C)c2ccccc2N2c3ccc(-c4cccc(-c5ccc6oc7ccccc7c(=O)c6c5)c4)cc3Oc3cccc1c32 ; CC1(C)c2ccccc2N2c3cccc(-c4ccc5oc6ccccc6c(=O)c5c4)c3Oc3cccc1c32]

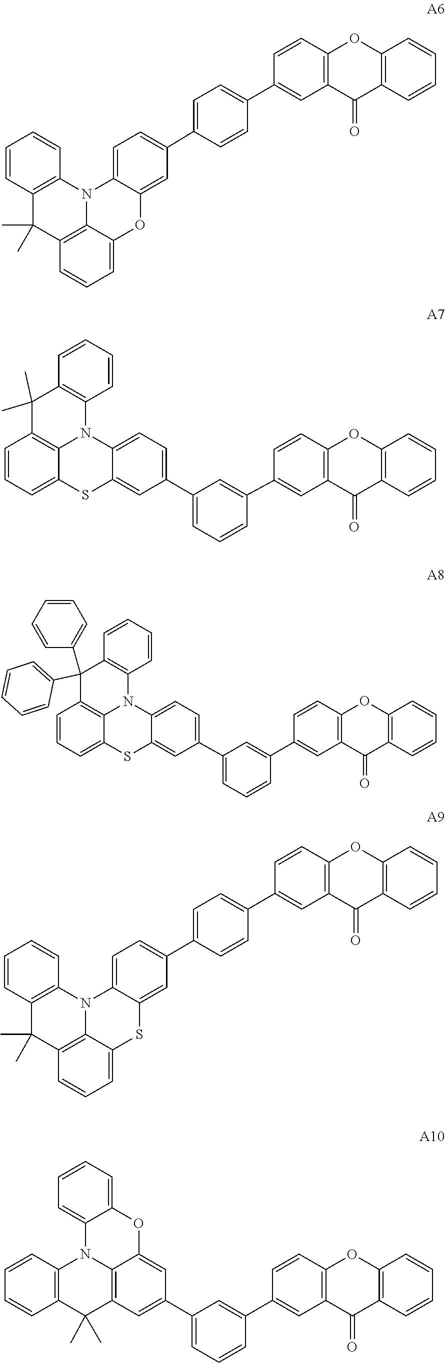 [SMILES: CC1(C)c2ccccc2N2c3ccc(-c4ccc(-c5ccc6oc7ccccc7c(=O)c6c5)cc4)cc3Oc3cccc1c32 ; CC1(C)c2ccccc2N2c3ccc(-c4cccc(-c5ccc6oc7ccccc7c(=O)c6c5)c4)cc3Sc3cccc1c32 ; O=c1c2ccccc2oc2ccc(-c3cccc(-c4ccc5c(c4)Sc4cccc6c4N5c4ccccc4C6(c4ccccc4)c4ccccc4)c3)cc12 ; CC1(C)c2ccccc2N2c3ccc(-c4ccc(-c5ccc6oc7ccccc7c(=O)c6c5)cc4)cc3Sc3cccc1c32 ; CC1(C)c2ccccc2N2c3ccccc3Oc3cc(-c4cccc(-c5ccc6oc7ccccc7c(=O)c6c5)c4)cc1c32]

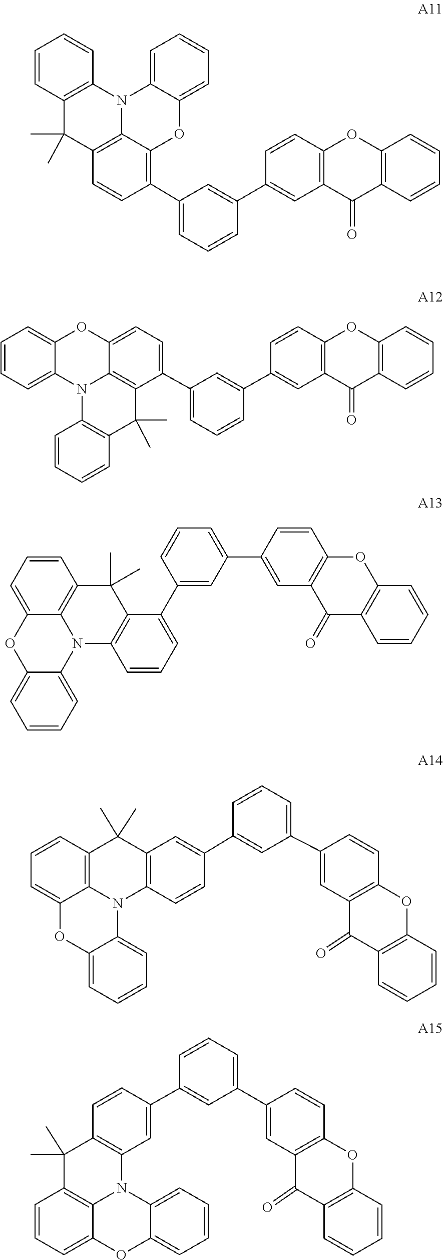 [SMILES: CC1(C)c2ccccc2N2c3ccccc3Oc3c(-c4cccc(-c5ccc6oc7ccccc7c(=O)c6c5)c4)ccc1c32 ; CC1(C)c2ccccc2N2c3ccccc3Oc3ccc(-c4cccc(-c5ccc6oc7ccccc7c(=O)c6c5)c4)c1c32 ; CC1(C)c2cccc3c2N(c2ccccc2O3)c2cccc(-c3cccc(-c4ccc5oc6ccccc6c(=O)c5c4)c3)c21 ; CC1(C)c2cc(-c3cccc(-c4ccc5oc6ccccc6c(=O)c5c4)c3)ccc2N2c3ccccc3Oc3cccc1c32 ; CC1(C)c2ccc(-c3cccc(-c4ccc5oc6ccccc6c(=O)c5c4)c3)cc2N2c3ccccc3Oc3cccc1c32]

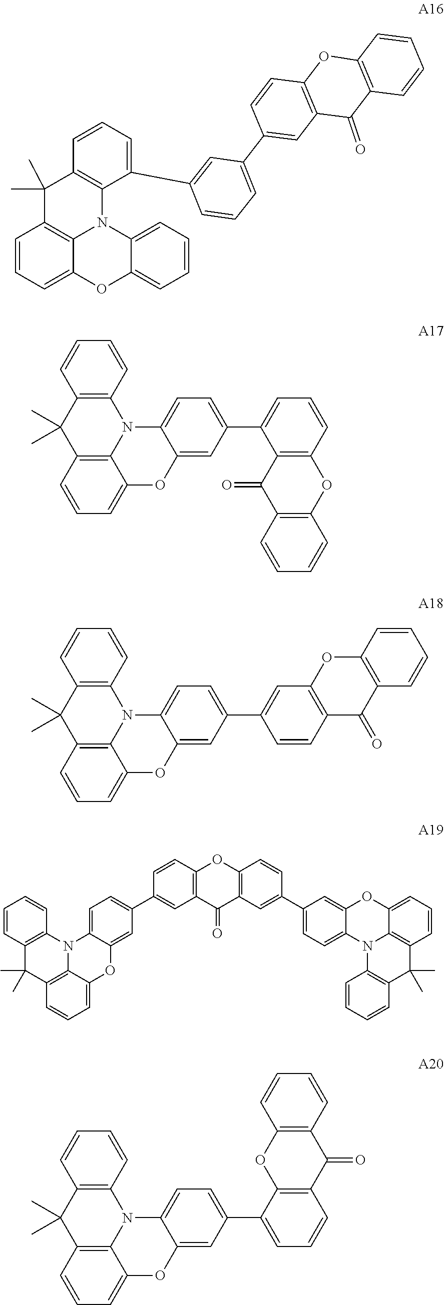

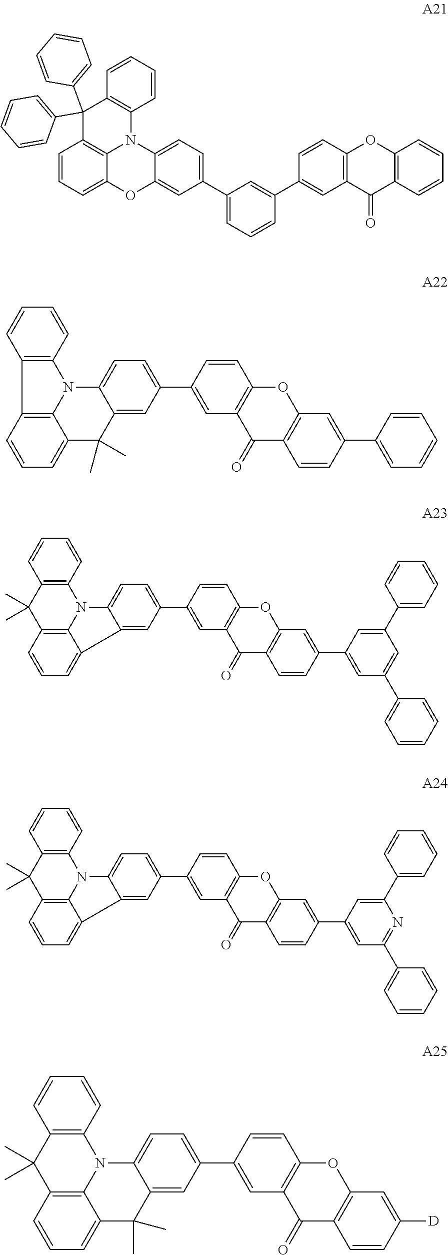 [SMILES: O=c1c2ccccc2oc2ccc(-c3cccc(-c4ccc5c(c4)Oc4cccc6c4N5c4ccccc4C6(c4ccccc4)c4ccccc4)c3)cc12 ; CC1(C)c2cc(-c3ccc4oc5cc(-c6ccccc6)ccc5c(=O)c4c3)ccc2-n2c3ccccc3c3cccc1c32 ; CC1(C)c2ccccc2-n2c3ccc(-c4ccc5oc6cc(-c7cc(-c8ccccc8)cc(-c8ccccc8)c7)ccc6c(=O)c5c4)cc3c3cccc1c32 ; CC1(C)c2ccccc2-n2c3ccc(-c4ccc5oc6cc(-c7cc(-c8ccccc8)nc(-c8ccccc8)c7)ccc6c(=O)c5c4)cc3c3cccc1c32 ; [2H]c1ccc2c(=O)c3cc(-c4ccc5c(c4)C(C)(C)c4cccc6c4N5c4ccccc4C6(C)C)ccc3oc2c1]

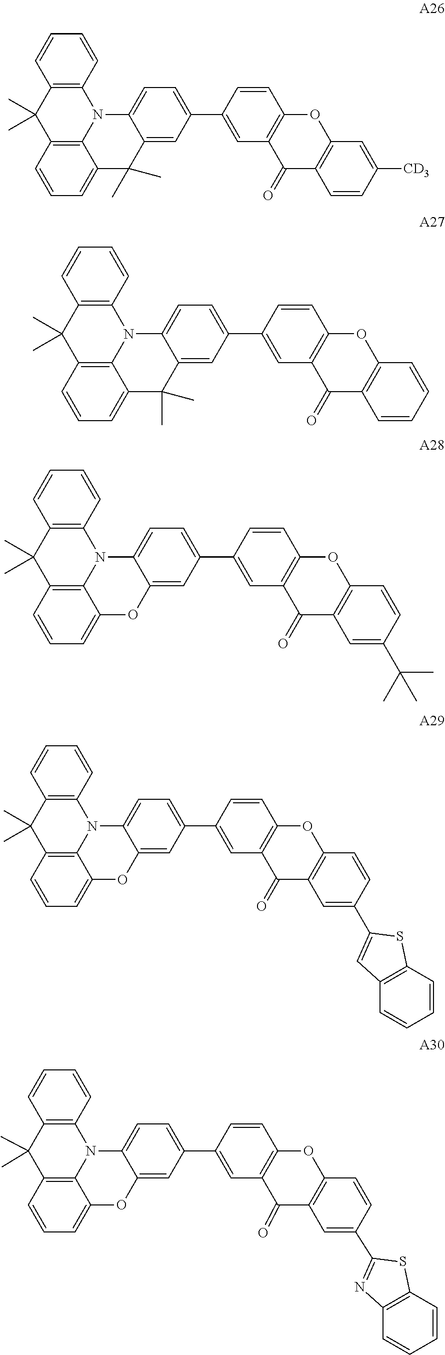

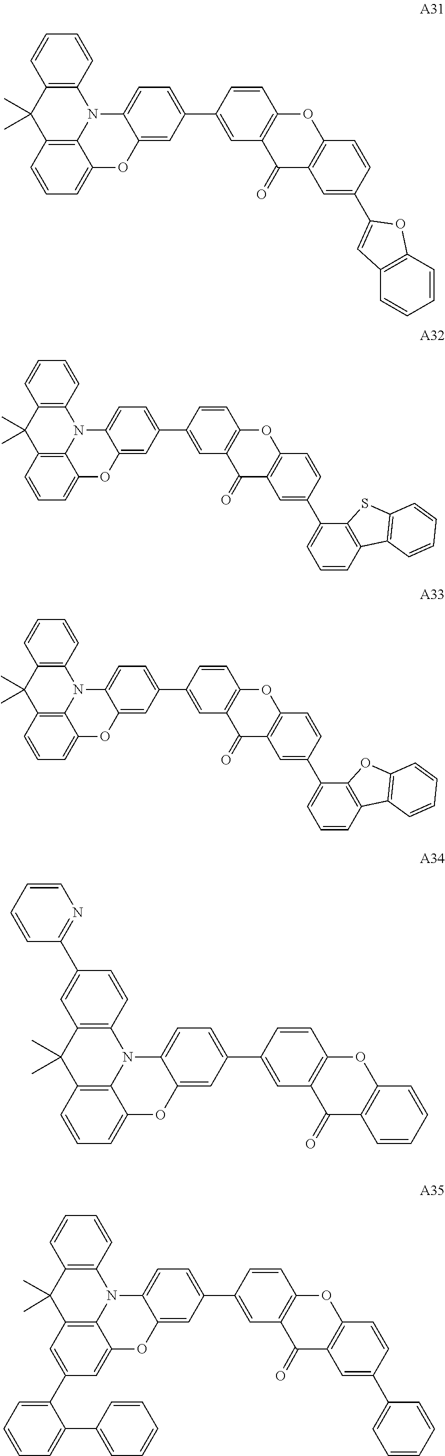 [SMILES: CC1(C)c2ccccc2N2c3ccc(-c4ccc5oc6ccc(-c7cc8ccccc8o7)cc6c(=O)c5c4)cc3Oc3cccc1c32 ; CC1(C)c2ccccc2N2c3ccc(-c4ccc5oc6ccc(-c7cccc8c7sc7ccccc78)cc6c(=O)c5c4)cc3Oc3cccc1c32 ; CC1(C)c2ccccc2N2c3ccc(-c4ccc5oc6ccc(-c7cccc8c7oc7ccccc78)cc6c(=O)c5c4)cc3Oc3cccc1c32 ; CC1(C)c2cc(-c3ccccn3)ccc2N2c3ccc(-c4ccc5oc6ccccc6c(=O)c5c4)cc3Oc3cccc1c32 ; CC1(C)c2ccccc2N2c3ccc(-c4ccc5oc6ccc(-c7ccccc7)cc6c(=O)c5c4)cc3Oc3cc(-c4ccccc4-c4ccccc4)cc1c32]

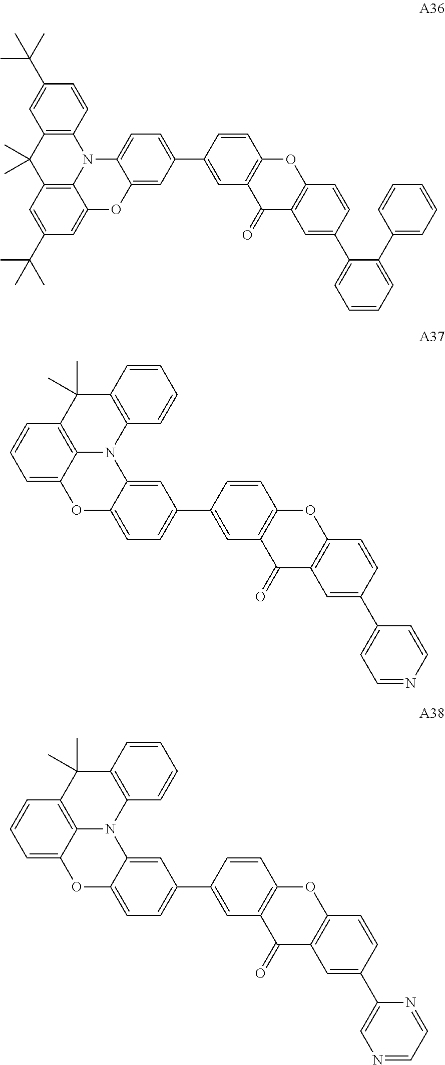 [SMILES: CC(C)(C)c1ccc2c(c1)C(C)(C)c1cc(C(C)(C)C)cc3c1N2c1ccc(-c2ccc4oc5ccc(-c6ccccc6-c6ccccc6)cc5c(=O)c4c2)cc1O3 ; CC1(C)c2ccccc2N2c3cc(-c4ccc5oc6ccc(-c7ccncc7)cc6c(=O)c5c4)ccc3Oc3cccc1c32 ; CC1(C)c2ccccc2N2c3cc(-c4ccc5oc6ccc(-c7cnccn7)cc6c(=O)c5c4)ccc3Oc3cccc1c32]

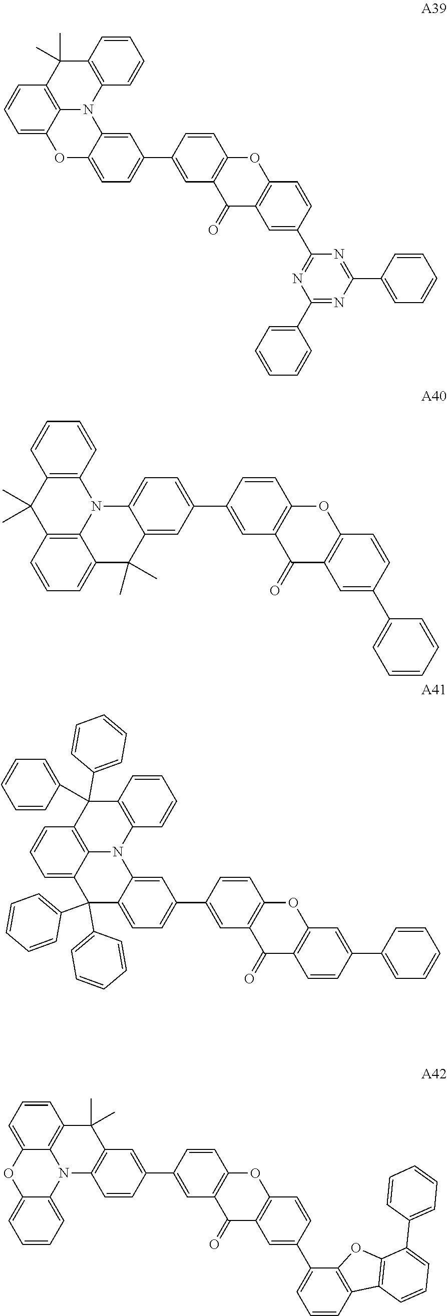 [SMILES: CC1(C)c2ccccc2N2c3cc(-c4ccc5oc6ccc(-c7nc(-c8ccccc8)nc(-c8ccccc8)n7)cc6c(=O)c5c4)ccc3Oc3cccc1c32 ; CC1(C)c2ccccc2N2c3ccc(-c4ccc5oc6ccc(-c7ccccc7)cc6c(=O)c5c4)cc3C(C)(C)c3cccc1c32 ; O=c1c2ccc(-c3ccccc3)cc2oc2ccc(-c3ccc4c(c3)N3c5ccccc5C(c5ccccc5)(c5ccccc5)c5cccc(c53)C4(c3ccccc3)c3ccccc3)cc12 ; CC1(C)c2cc(-c3ccc4oc5ccc(-c6cccc7c6oc6c(-c8ccccc8)cccc67)cc5c(=O)c4c3)ccc2N2c3ccccc3Oc3cccc1c32]

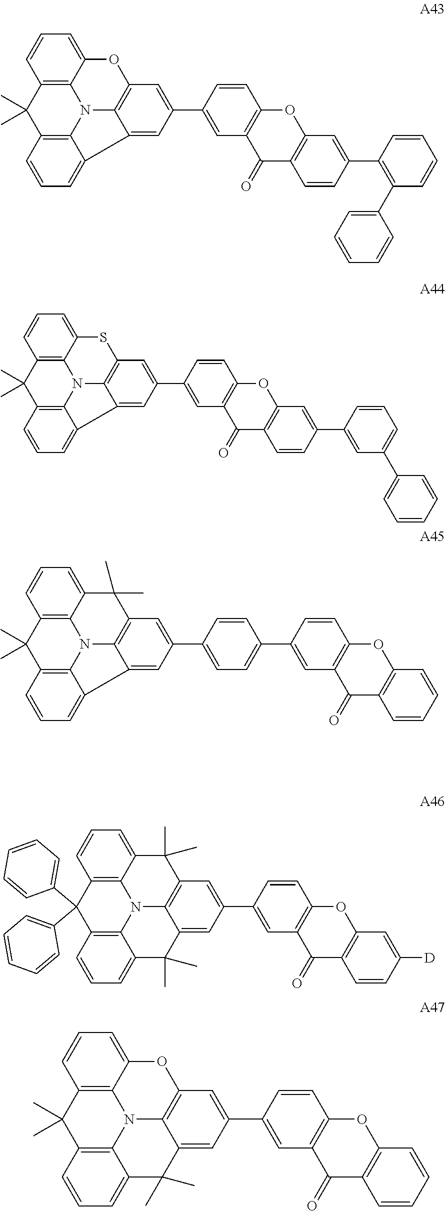 [SMILES: CC1(C)c2cccc3c2-n2c4c(cc(-c5ccc6oc7cc(-c8ccccc8-c8ccccc8)ccc7c(=O)c6c5)cc4c4cccc1c42)O3 ; CC1(C)c2cccc3c2-n2c4c(cc(-c5ccc6oc7cc(-c8cccc(-c9ccccc9)c8)ccc7c(=O)c6c5)cc4c4cccc1c42)S3 ; CC1(C)c2cccc3c2-n2c4c1cccc4c1cc(-c4ccc(-c5ccc6oc7ccccc7c(=O)c6c5)cc4)cc(c12)C3(C)C ; [2H]c1ccc2c(=O)c3cc(-c4cc5c6c(c4)C(C)(C)c4cccc7c4N6c4c(cccc4C7(c4ccccc4)c4ccccc4)C5(C)C)ccc3oc2c1 ; CC1(C)c2cccc3c2N2c4c(cc(-c5ccc6oc7ccccc7c(=O)c6c5)cc4C(C)(C)c4cccc1c42)O3]

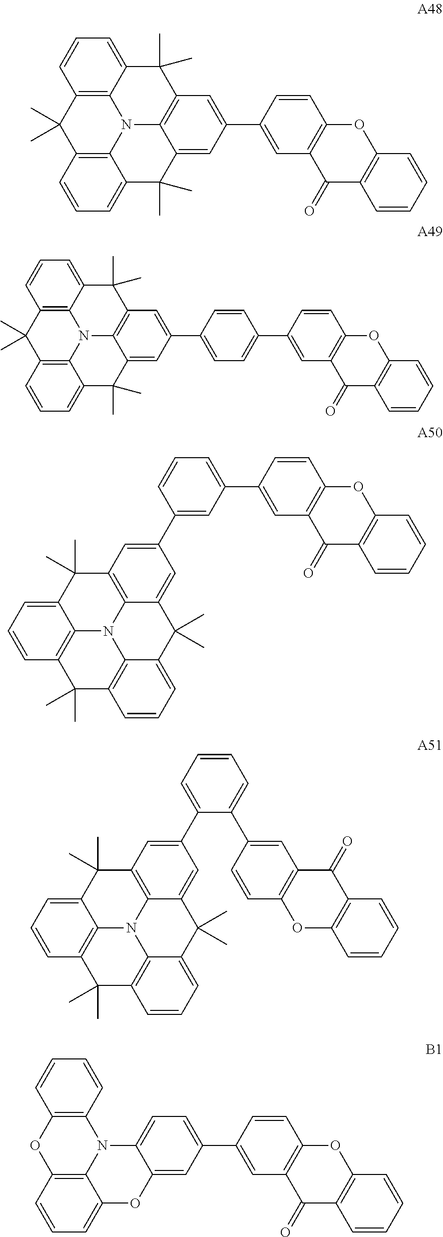

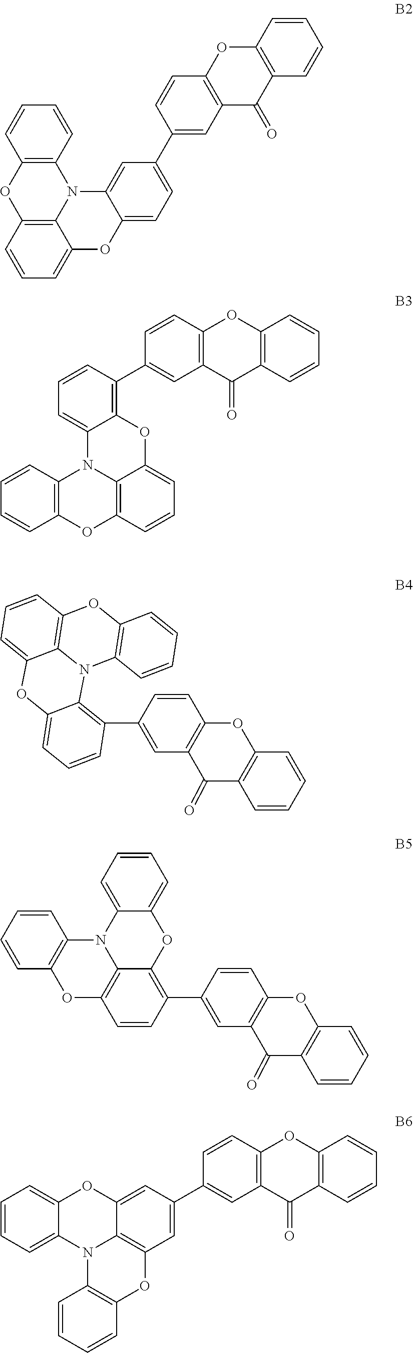 [SMILES: O=c1c2ccccc2oc2ccc(-c3ccc4c(c3)N3c5ccccc5Oc5cccc(c53)O4)cc12 ; O=c1c2ccccc2oc2ccc(-c3cccc4c3Oc3cccc5c3N4c3ccccc3O5)cc12 ; O=c1c2ccccc2oc2ccc(-c3cccc4c3N3c5ccccc5Oc5cccc(c53)O4)cc12 ; O=c1c2ccccc2oc2ccc(-c3ccc4c5c3Oc3ccccc3N5c3ccccc3O4)cc12 ; O=c1c2ccccc2oc2ccc(-c3cc4c5c(c3)Oc3ccccc3N5c3ccccc3O4)cc12]

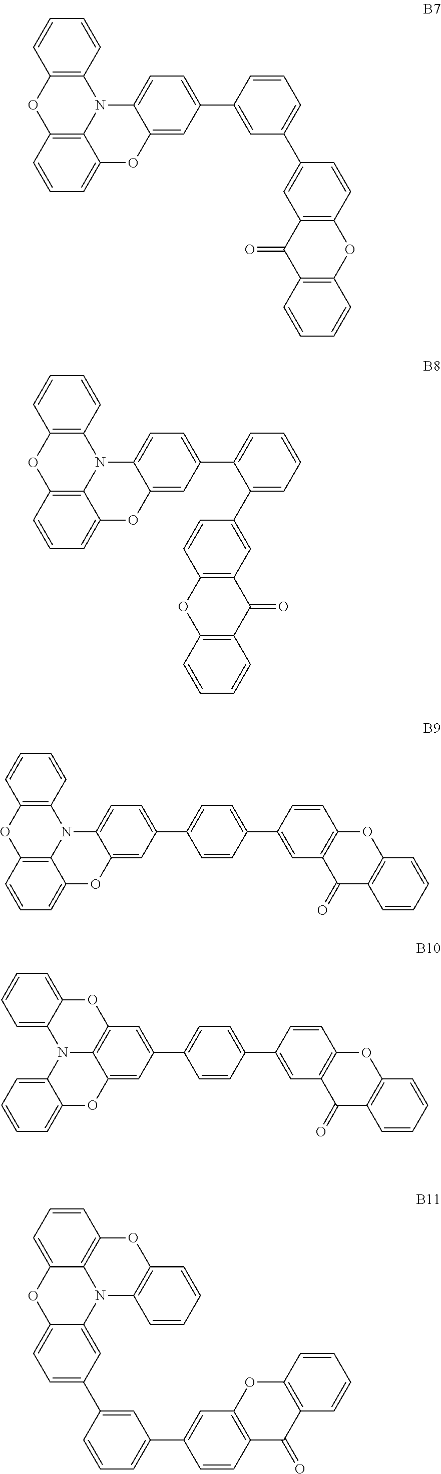 [SMILES: O=c1c2ccccc2oc2ccc(-c3cccc(-c4ccc5c(c4)Oc4cccc6c4N5c4ccccc4O6)c3)cc12 ; O=c1c2ccccc2oc2ccc(-c3ccccc3-c3ccc4c(c3)Oc3cccc5c3N4c3ccccc3O5)cc12 ; O=c1c2ccccc2oc2ccc(-c3ccc(-c4ccc5c(c4)Oc4cccc6c4N5c4ccccc4O6)cc3)cc12 ; O=c1c2ccccc2oc2ccc(-c3ccc(-c4cc5c6c(c4)Oc4ccccc4N6c4ccccc4O5)cc3)cc12 ; O=c1c2ccccc2oc2cc(-c3cccc(-c4ccc5c(c4)N4c6ccccc6Oc6cccc(c64)O5)c3)ccc12]

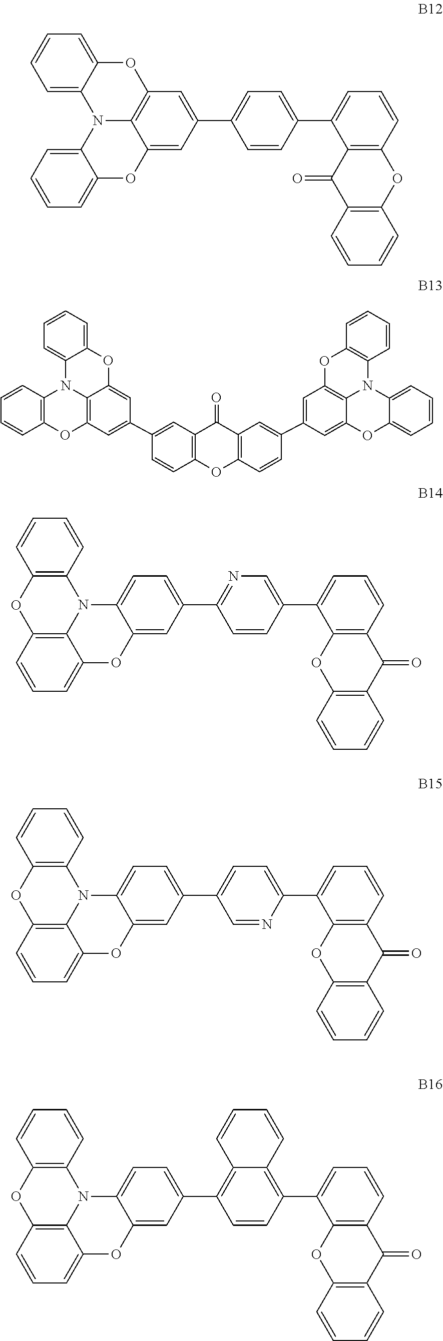 [SMILES: O=c1c2ccccc2oc2cccc(-c3ccc(-c4cc5c6c(c4)Oc4ccccc4N6c4ccccc4O5)cc3)c12 ; O=c1c2cc(-c3cc4c5c(c3)Oc3ccccc3N5c3ccccc3O4)ccc2oc2ccc(-c3cc4c5c(c3)Oc3ccccc3N5c3ccccc3O4)cc12 ; O=c1c2ccccc2oc2c(-c3ccc(-c4ccc5c(c4)Oc4cccc6c4N5c4ccccc4O6)nc3)cccc12 ; O=c1c2ccccc2oc2c(-c3ccc(-c4ccc5c(c4)Oc4cccc6c4N5c4ccccc4O6)cn3)cccc12 ; O=c1c2ccccc2oc2c(-c3ccc(-c4ccc5c(c4)Oc4cccc6c4N5c4ccccc4O6)c4ccccc34)cccc12]

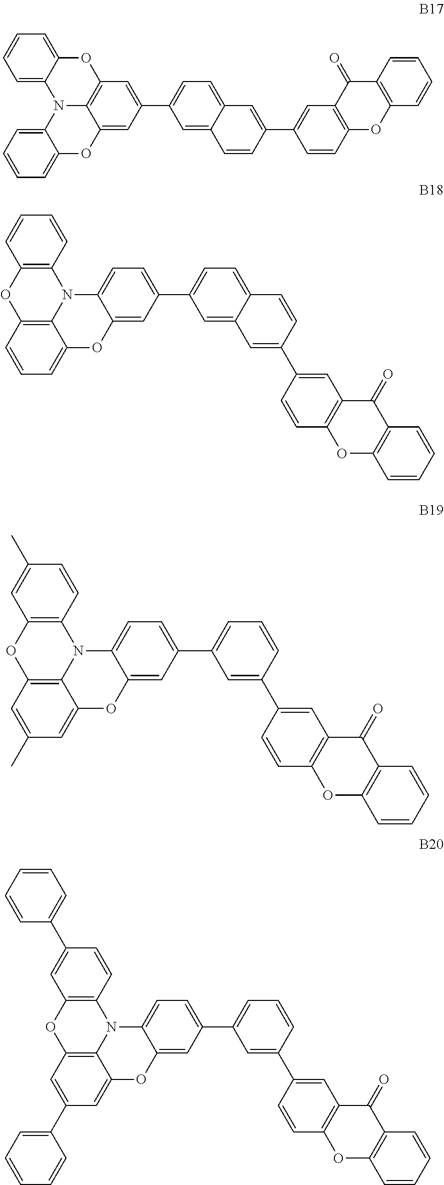

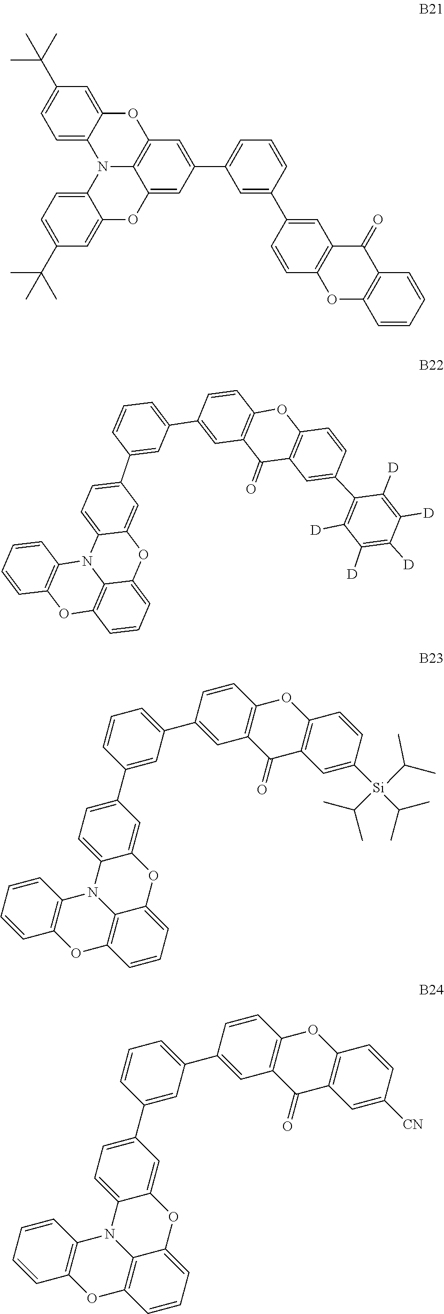 [SMILES: CC(C)(C)c1ccc2c(c1)Oc1cc(-c3cccc(-c4ccc5oc6ccccc6c(=O)c5c4)c3)cc3c1N2c1ccc(C(C)(C)C)cc1O3 ; [2H]c1c([2H])c([2H])c(-c2ccc3oc4ccc(-c5cccc(-c6ccc7c(c6)Oc6cccc8c6N7c6ccccc6O8)c5)cc4c(=O)c3c2)c([2H])c1[2H] ; CC(C)[Si](c1ccc2oc3ccc(-c4cccc(-c5ccc6c(c5)Oc5cccc7c5N6c5ccccc5O7)c4)cc3c(=O)c2c1)(C(C)C)C(C)C ; N#Cc1ccc2oc3ccc(-c4cccc(-c5ccc6c(c5)Oc5cccc7c5N6c5ccccc5O7)c4)cc3c(=O)c2c1]

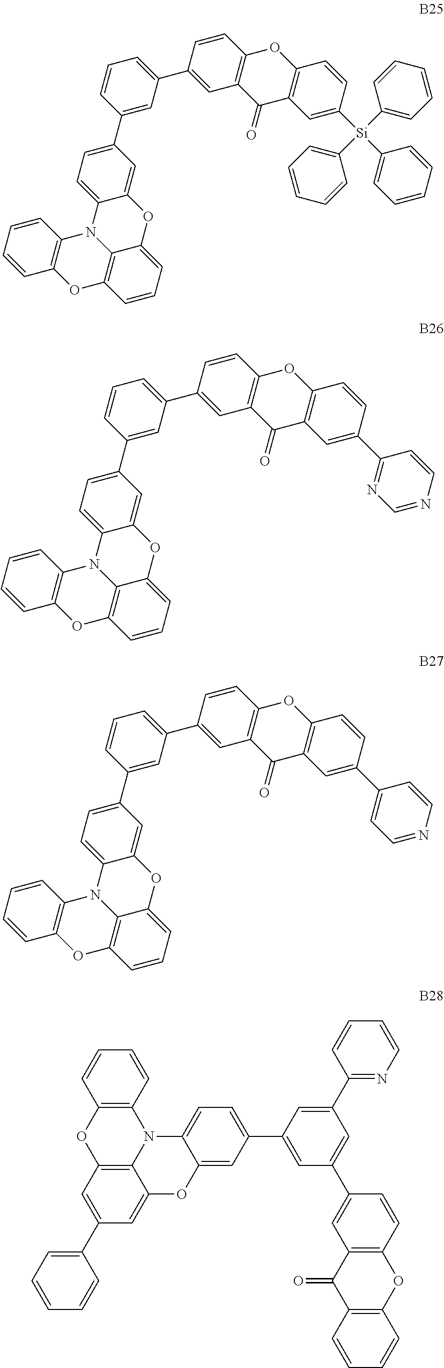 [SMILES: O=c1c2cc(-c3cccc(-c4ccc5c(c4)Oc4cccc6c4N5c4ccccc4O6)c3)ccc2oc2ccc([Si](c3ccccc3)(c3ccccc3)c3ccccc3)cc12 ; O=c1c2cc(-c3cccc(-c4ccc5c(c4)Oc4cccc6c4N5c4ccccc4O6)c3)ccc2oc2ccc(-c3ccncn3)cc12 ; O=c1c2cc(-c3ccncc3)ccc2oc2ccc(-c3cccc(-c4ccc5c(c4)Oc4cccc6c4N5c4ccccc4O6)c3)cc12 ; O=c1c2ccccc2oc2ccc(-c3cc(-c4ccc5c(c4)Oc4cc(-c6ccccc6)cc6c4N5c4ccccc4O6)cc(-c4ccccn4)c3)cc12]

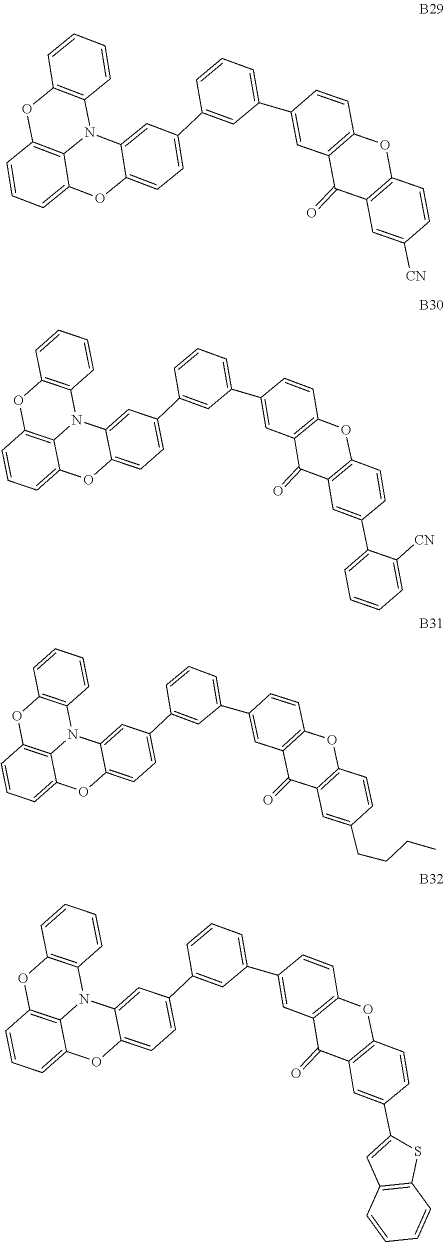 [SMILES: N#Cc1ccc2oc3ccc(-c4cccc(-c5ccc6c(c5)N5c7ccccc7Oc7cccc(c75)O6)c4)cc3c(=O)c2c1 ; N#Cc1ccccc1-c1ccc2oc3ccc(-c4cccc(-c5ccc6c(c5)N5c7ccccc7Oc7cccc(c75)O6)c4)cc3c(=O)c2c1 ; CCCCc1ccc2oc3ccc(-c4cccc(-c5ccc6c(c5)N5c7ccccc7Oc7cccc(c75)O6)c4)cc3c(=O)c2c1 ; O=c1c2cc(-c3cccc(-c4ccc5c(c4)N4c6ccccc6Oc6cccc(c64)O5)c3)ccc2oc2ccc(-c3cc4ccccc4s3)cc12]

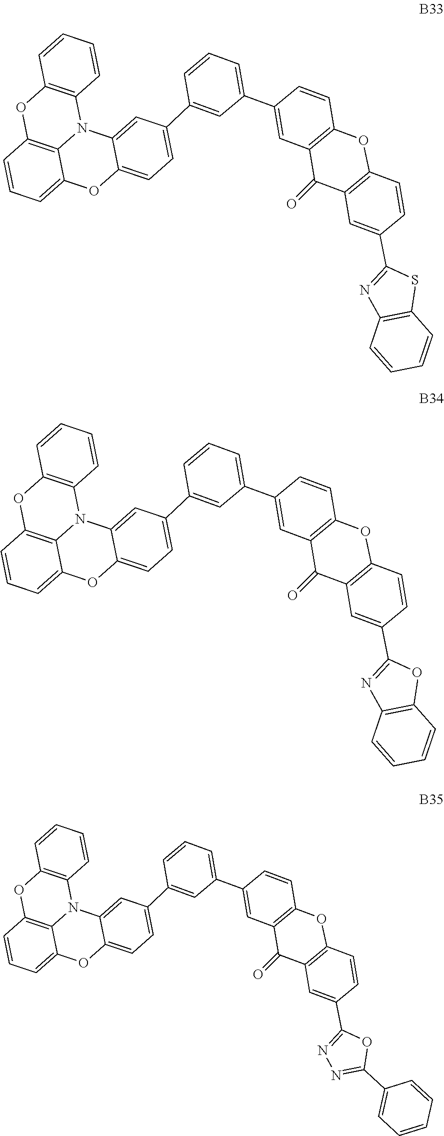 [SMILES: O=c1c2cc(-c3cccc(-c4ccc5c(c4)N4c6ccccc6Oc6cccc(c64)O5)c3)ccc2oc2ccc(-c3nc4ccccc4s3)cc12 ; O=c1c2cc(-c3cccc(-c4ccc5c(c4)N4c6ccccc6Oc6cccc(c64)O5)c3)ccc2oc2ccc(-c3nc4ccccc4o3)cc12 ; O=c1c2cc(-c3cccc(-c4ccc5c(c4)N4c6ccccc6Oc6cccc(c64)O5)c3)ccc2oc2ccc(-c3nnc(-c4ccccc4)o3)cc12]

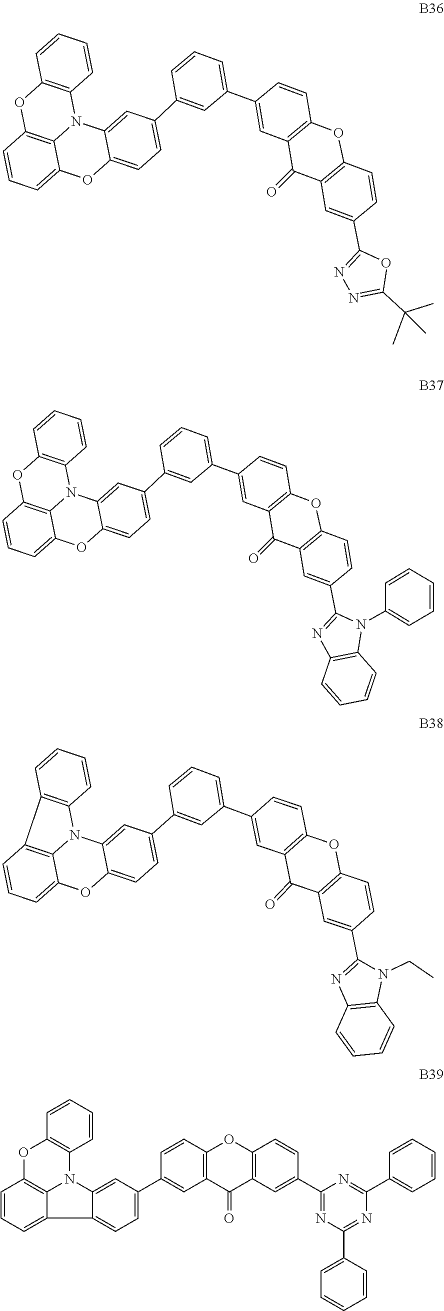 [SMILES: CC(C)(C)c1nnc(-c2ccc3oc4ccc(-c5cccc(-c6ccc7c(c6)N6c8ccccc8Oc8cccc(c86)O7)c5)cc4c(=O)c3c2)o1 ; O=c1c2cc(-c3cccc(-c4ccc5c(c4)N4c6ccccc6Oc6cccc(c64)O5)c3)ccc2oc2ccc(-c3nc4ccccc4n3-c3ccccc3)cc12 ; CCn1c(-c2ccc3oc4ccc(-c5cccc(-c6ccc7c(c6)-n6c8ccccc8c8cccc(c86)O7)c5)cc4c(=O)c3c2)nc2ccccc21 ; O=c1c2cc(-c3ccc4c5cccc6c5n(c4c3)-c3ccccc3O6)ccc2oc2ccc(-c3nc(-c4ccccc4)nc(-c4ccccc4)n3)cc12]

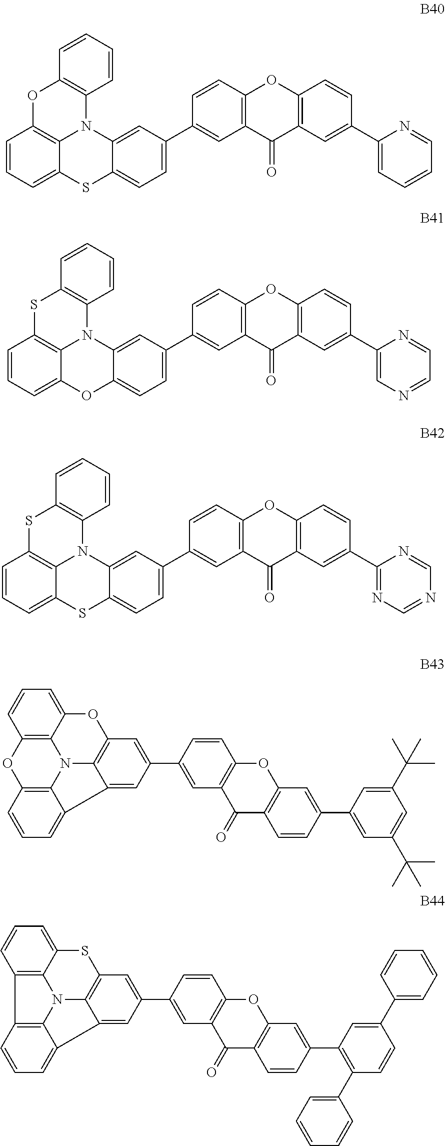 [SMILES: O=c1c2cc(-c3ccc4c(c3)N3c5ccccc5Oc5cccc(c53)S4)ccc2oc2ccc(-c3ccccn3)cc12 ; O=c1c2cc(-c3ccc4c(c3)N3c5ccccc5Sc5cccc(c53)O4)ccc2oc2ccc(-c3cnccn3)cc12 ; O=c1c2cc(-c3ccc4c(c3)N3c5ccccc5Sc5cccc(c53)S4)ccc2oc2ccc(-c3ncncn3)cc12 ; CC(C)(C)c1cc(-c2ccc3c(=O)c4cc(-c5cc6c7c(c5)c5cccc8c5n7-c5c(cccc5O6)O8)ccc4oc3c2)cc(C(C)(C)C)c1 ; O=c1c2ccc(-c3cc(-c4ccccc4)ccc3-c3ccccc3)cc2oc2ccc(-c3cc4c5c(c3)c3cccc6c7cccc(c7n5c63)S4)cc12]

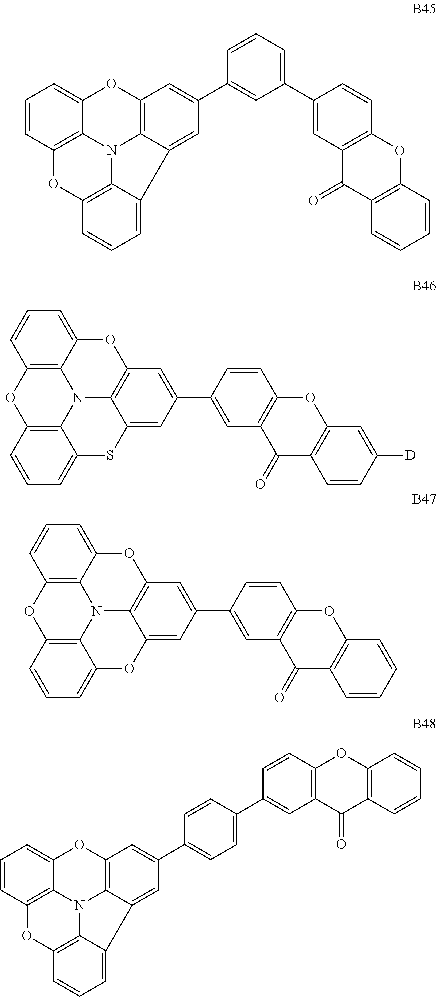

Among the above exemplary compounds, the exemplary compounds belonging to the group A are compounds in which at least one of X₁ and X₂ is CR₂₀R₂₁. These compounds have a substituent at CR₂₀R₂₁, have the substituent in the direction perpendicular to the in-plane direction of the electron-donating moiety, and can therefore particularly suppress the overlap between electron-donating moieties. Thus, these compounds have particularly good amorphous properties.

Among the above exemplary compounds, the exemplary compounds belonging to the group B are compounds in which at least one of X₁ and X₂ contains an oxygen atom or a sulfur atom. These compounds contain an oxygen atom or a sulfur atom in the electron-donating moiety. Abundant lone pairs in these atoms can enhance charge transport ability and make it particularly easy to adjust the carrier balance of the compounds.

Furthermore, the compound according to the present embodiment can be used in a light-emitting layer in an organic light-emitting element under the following conditions.

(6) The compound according to the present embodiment constitutes 50% to 99% by mass as a host material in a light-emitting layer.
(7) The compound according to the present embodiment constitutes 10% to 49% by mass as an assist material in a light-emitting layer.
(8) A light-emitting material to be mixed with the compound according to the present embodiment in a light-emitting layer is a phosphorescent material with a tricyclic or higher polycyclic fused ring in a ligand thereof.

These conditions are described below.

(6) The compound according to the present embodiment constitutes 50% to 99% by mass as a host material in a light-emitting layer.

Due to its enhanced amorphous properties, the organic compound according to the present embodiment is a material suitable for a host material for a light-emitting layer and can constitute 50% or more by mass. Even when constituting 99% by mass, the organic compound is rarely crystallized and sufficiently exhibits a function with good characteristics.

This is due to the structural characteristics of the organic compound according to the present embodiment. The organic compound according to the present embodiment characteristically has a structure in which triphenylamine is cross-linked to decrease the flatness of the electron-donating moiety. Thus, it is possible to provide a light-emitting element with good characteristics in which the compound is less likely to aggregate and crystal grain boundaries associated with molecular aggregation are rarely formed even while the organic light-emitting element is driven. The organic compound has the electron-donating moiety and the electron-withdrawing moiety and has bipolar nature. Thus, the organic compound is useful as a bipolar host.

(7) The compound according to the present embodiment constitutes 10% to 49% by mass as an assist material in a light-emitting layer.

When the organic compound according to the present embodiment is used for a light-emitting layer, the organic compound may be used as an assist material to improve the film properties of the light-emitting layer. The organic compound used as an assist material may constitute 10% to 49% by mass. Furthermore, exploiting its bipolar nature, the organic compound according to the present embodiment can also be used as a hole trapping assist or an electron trapping assist.

As described above in (6), when the organic compound according to the present embodiment is used as a host material in a light-emitting layer, the organic compound preferably constitutes 50% to 99% by mass. It can therefore be said that the organic compound according to the present embodiment can constitute 10% to 99% by mass as a host material or an assist material in a light-emitting layer.

(8) A light-emitting material to be mixed with the compound according to the present embodiment in a light-emitting layer is a phosphorescent material with a tricyclic or higher polycyclic fused ring in a ligand thereof.

The organic compound according to the present embodiment is a compound with a structure in which triphenylamine is cross-linked and with a xanthone skeleton. Thus, a phosphorescent material used with the organic compound according to the present embodiment in a light-emitting layer can have a structure in which π-conjugation of a ligand is extended. More specifically, the ligand can have a tricyclic or higher polycyclic fused ring. This is because, as in host materials, a structure with high flatness allows moieties with high flatness to approach each other through interaction. More specifically, a flat moiety of a host material easily approaches a ligand of an organometallic complex. This can be expected to decrease the intermolecular distance between the host material and the organometallic complex.

It is known that triplet energy utilized in a phosphorescent element is transferred by the Dexter mechanism. The Dexter mechanism includes energy transfer by intermolecular contact. More specifically, the intermolecular distance between a host material and a guest material is shortened for efficient energy transfer from the host material to the guest material.

In the present embodiment, the use of a highly flat organometallic complex with a tricyclic or higher polycyclic fused ring in the structure of a ligand decreases the intermolecular distance between the organometallic complex and a host material, which is the organic compound according to the present embodiment, and facilitates more efficient energy transfer from the host material to the organometallic complex. Consequently, an efficient organic light-emitting element can be provided.

A tricyclic or higher polycyclic fused-ring structure with high flatness in a ligand thereof refers to triphenylene, phenanthrene, fluorene, benzofluorene, dibenzofuran, dibenzothiophene, benzoisoquinoline, or naphthoisoquinoline. Thus, using an organometallic complex with at least one of these structures in a ligand as a light-emitting material, the organic compound according to the present embodiment can provide a more efficient light-emitting element. More specifically, it is a compound represented by the following general formula [2]:

Ir(L)ₘ(L′)ₙ(L″)ᵣ [2]

wherein L, L′, and L″ independently denote a different bidentate ligand.

m is an integer in the range of 1 to 3, and n and r are independently selected from 0 to 2, provided that m+n+r=3.

The partial structure Ir(L)ₘ is represented by any one of the following general formulae [Ir-1] to [Ir-11].

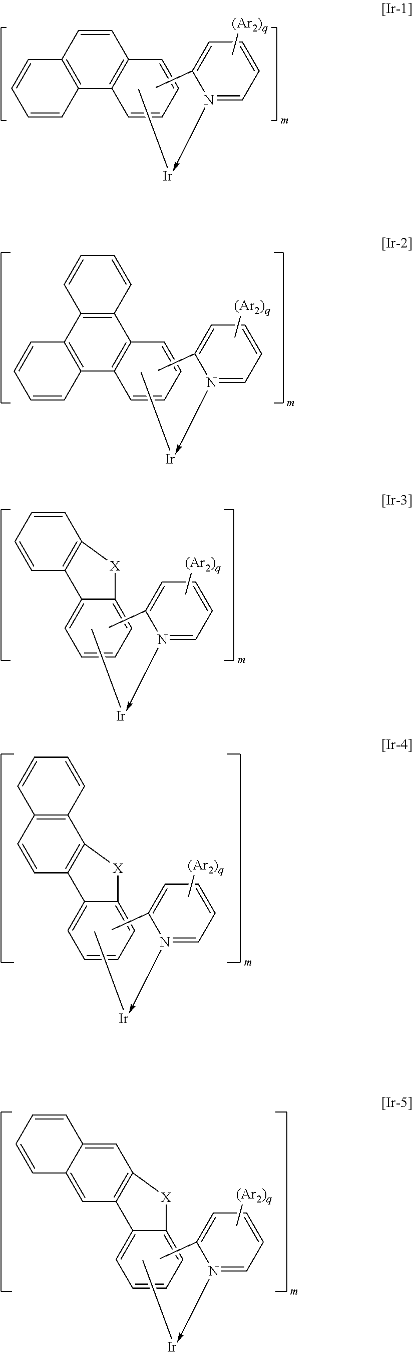

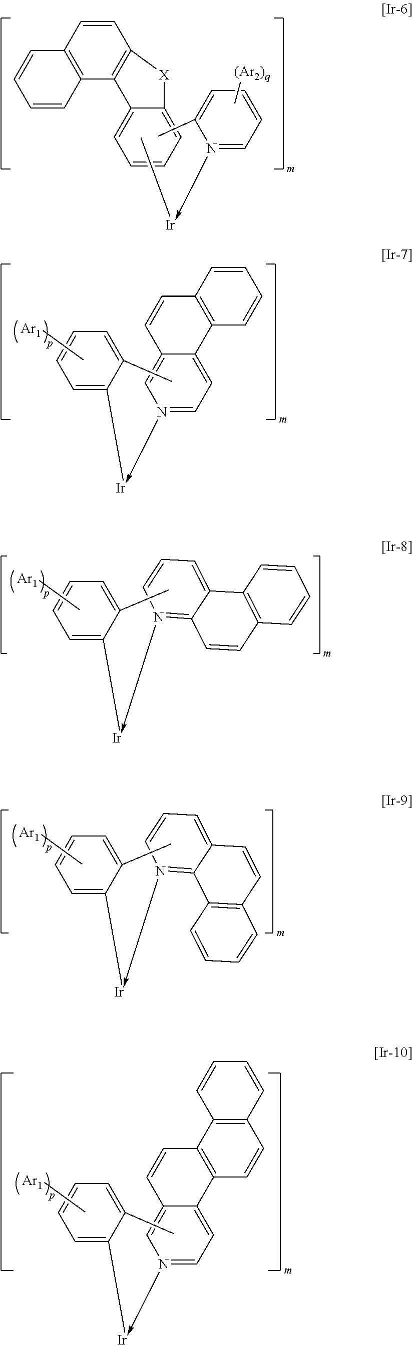

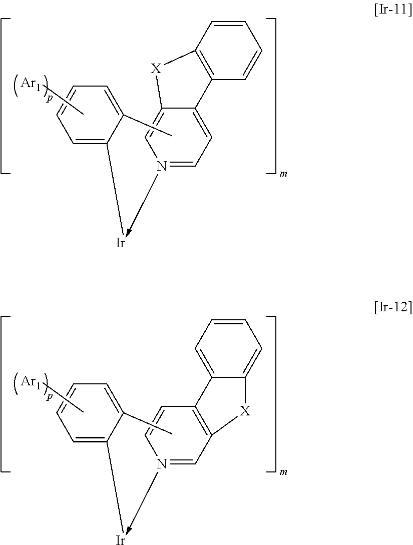

In the formulae [Ir-1] to [Ir-11], Ar₁ and Ar₂ independently denote a substituted or unsubstituted aryl group or a substituted or unsubstituted heterocyclic group, p and q denote an integer in the range of 0 to 4, and X is selected from the group consisting of an oxygen atom, a sulfur atom, a substituted or unsubstituted carbon atom, and a substituted or unsubstituted nitrogen atom.

L may further have a deuterium atom, a fluorine atom, a substituted or unsubstituted alkyl group, a deuterium-substituted alkyl group, an alkoxy group, a substituted or unsubstituted silyl group, a cyano group, a substituted or unsubstituted aryl group, and/or a substituted or unsubstituted heterocyclic group.

Specific examples of an organometallic complex that can be suitably used with the compound according to the present embodiment are described below. However, the present embodiments are not limited to these examples.

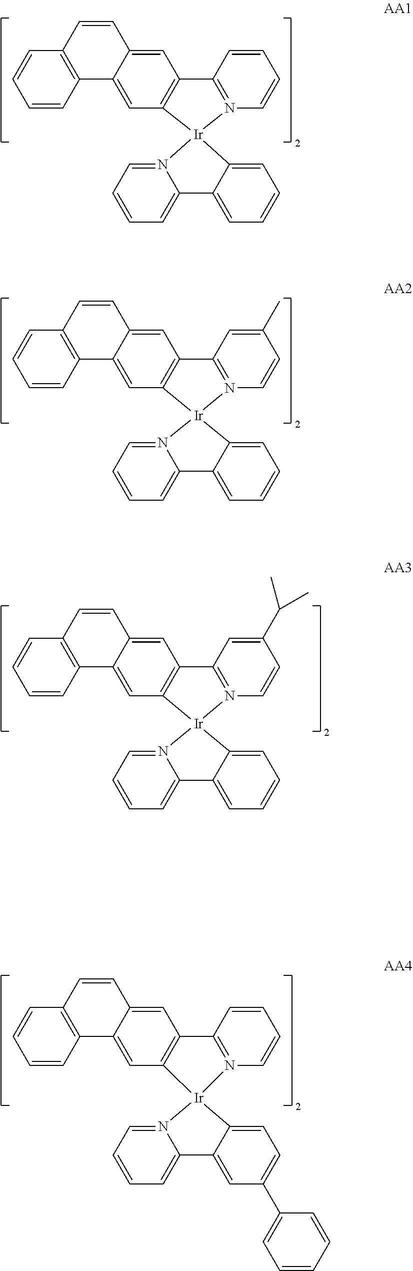

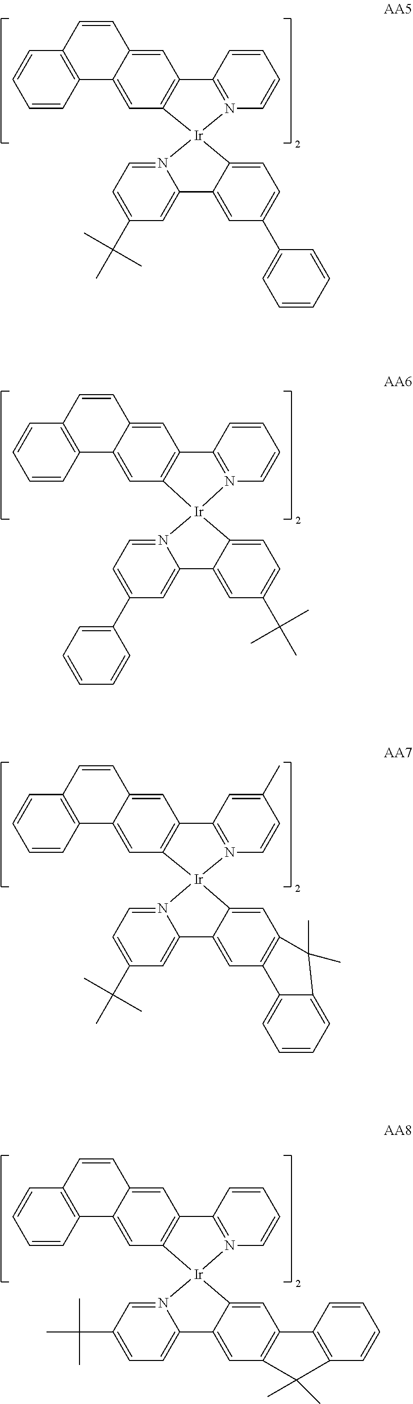

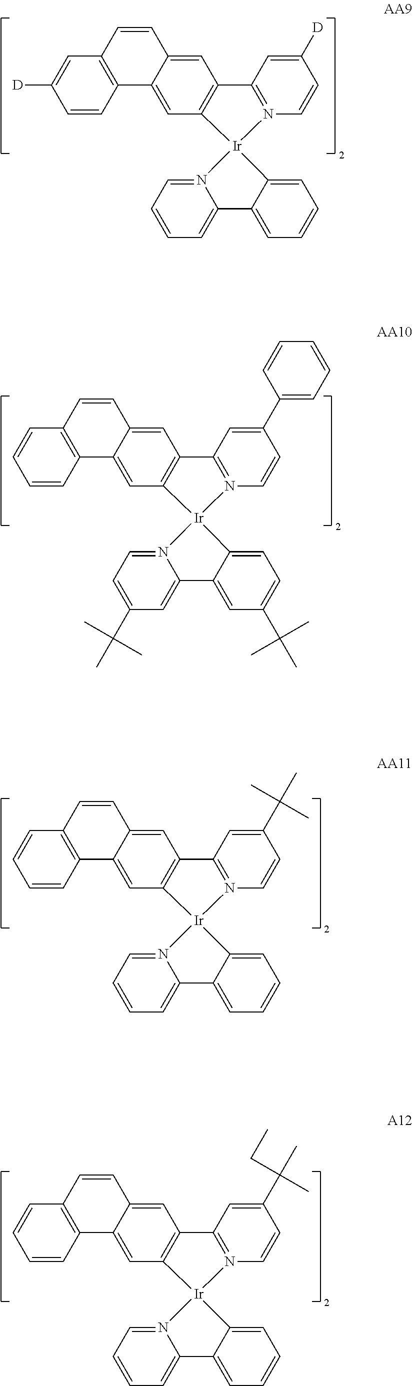

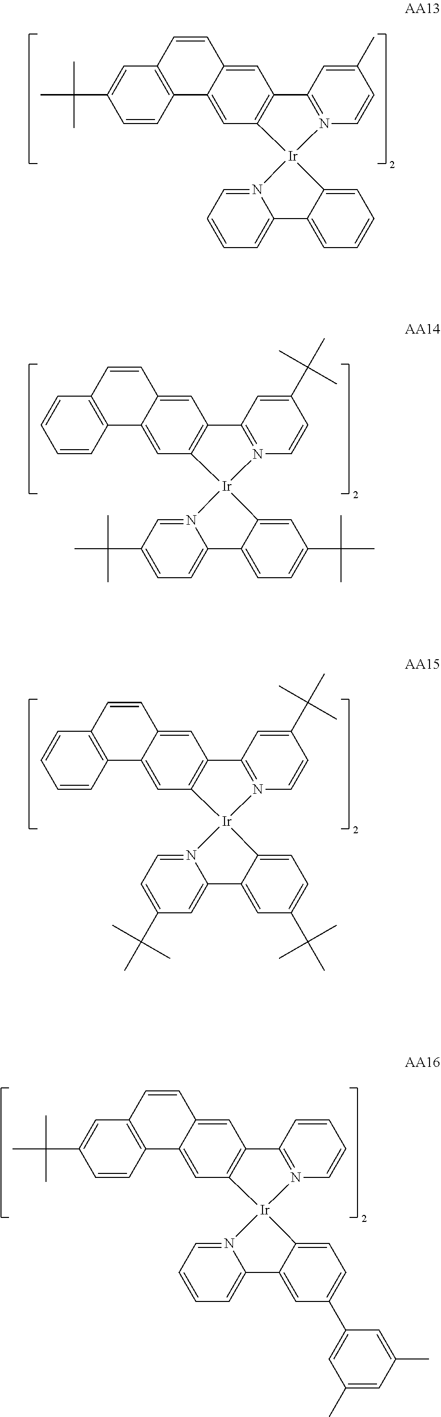

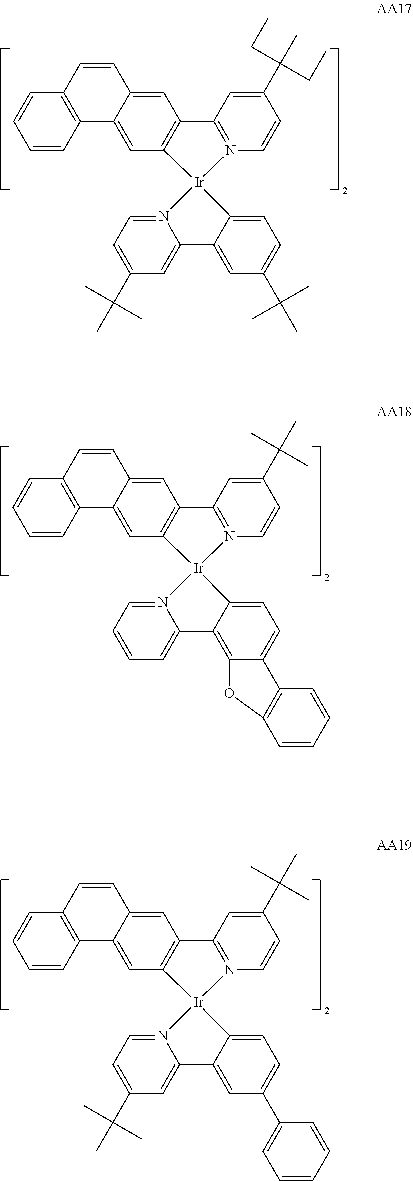

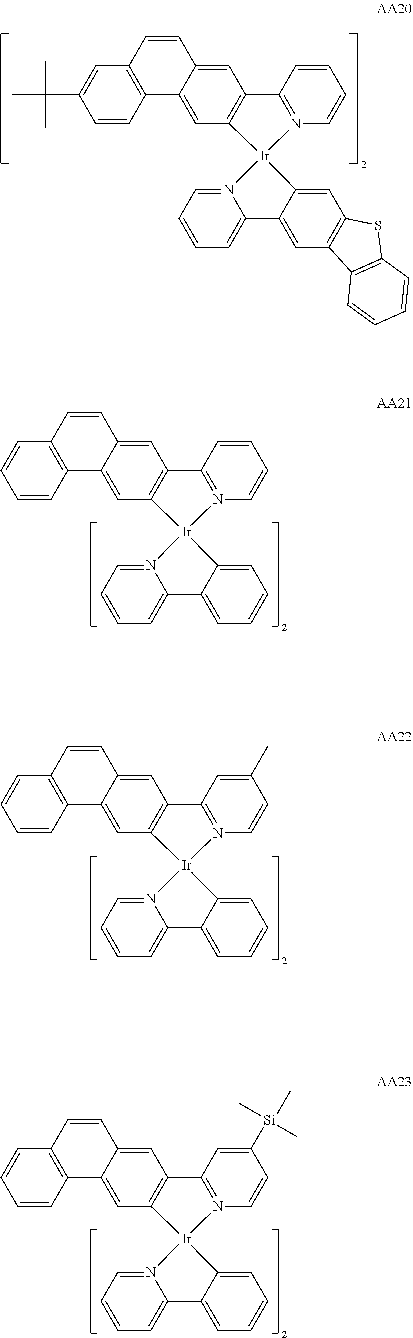

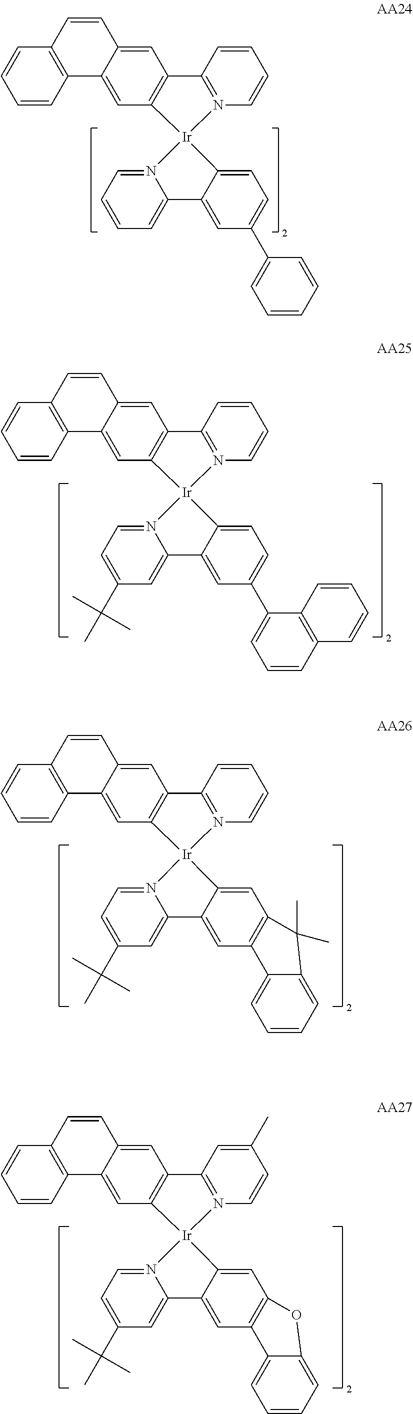

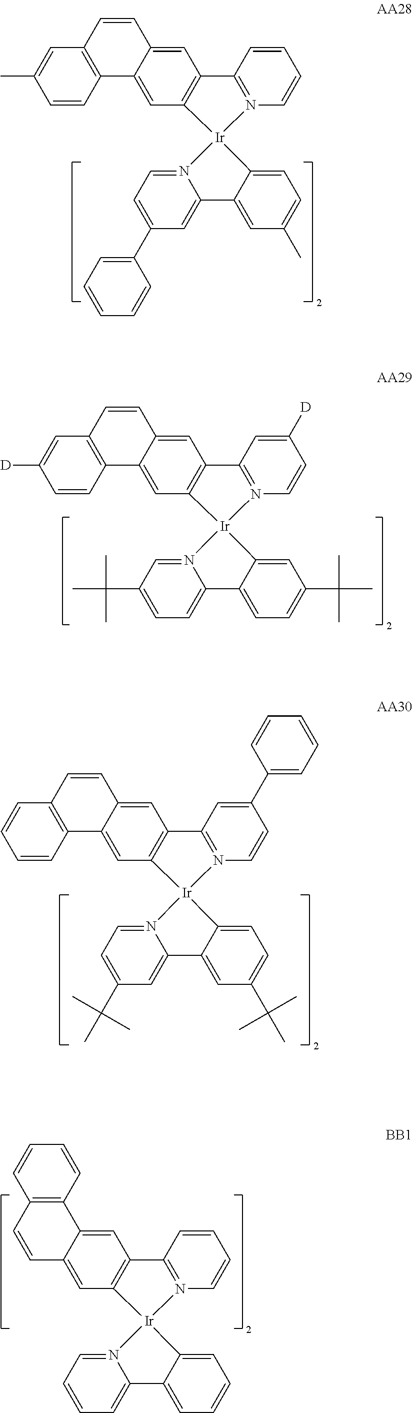

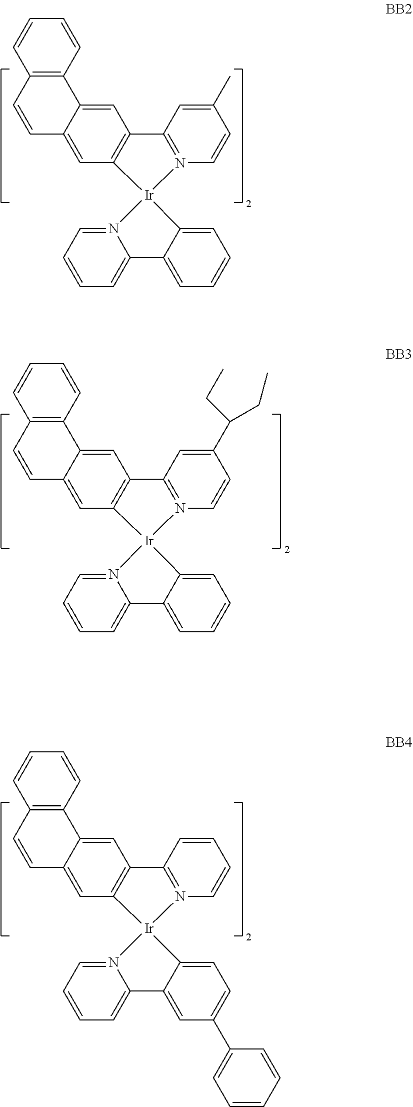

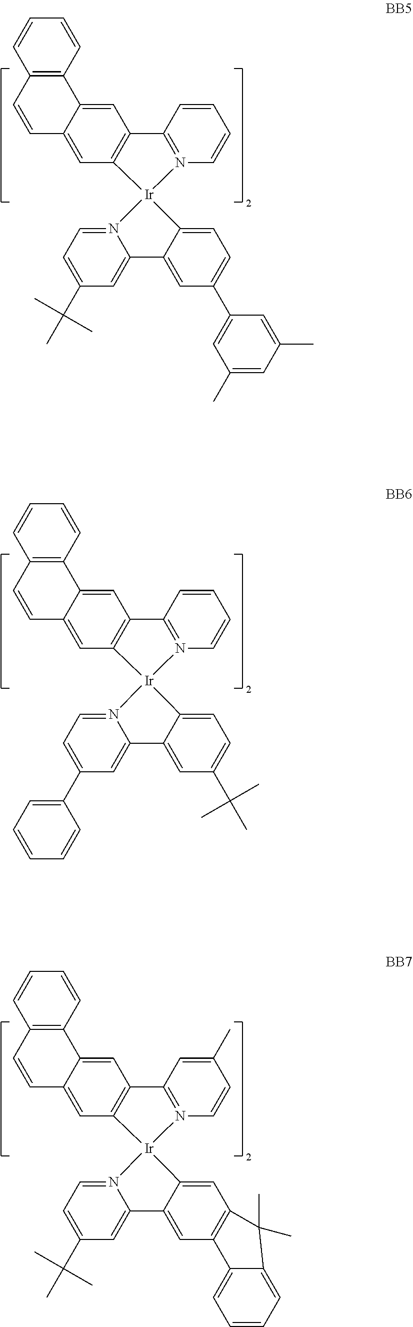

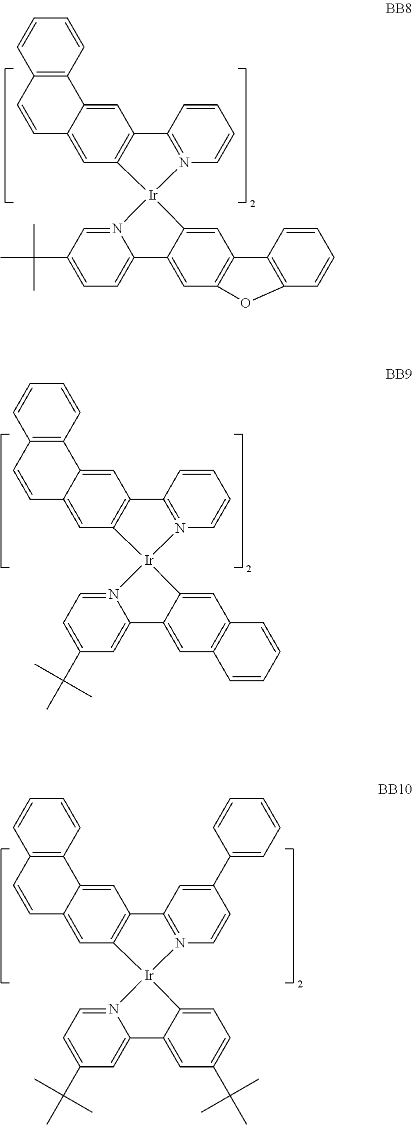

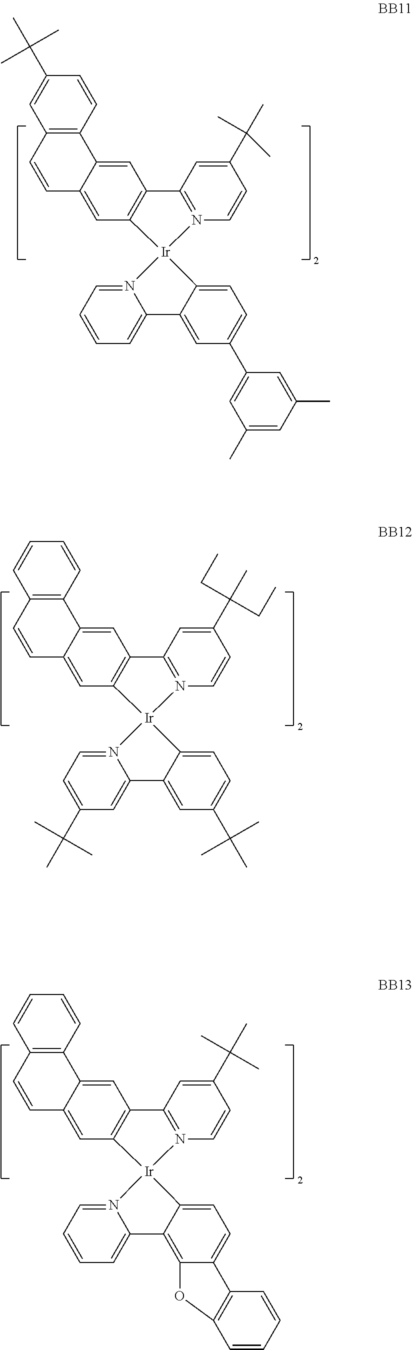

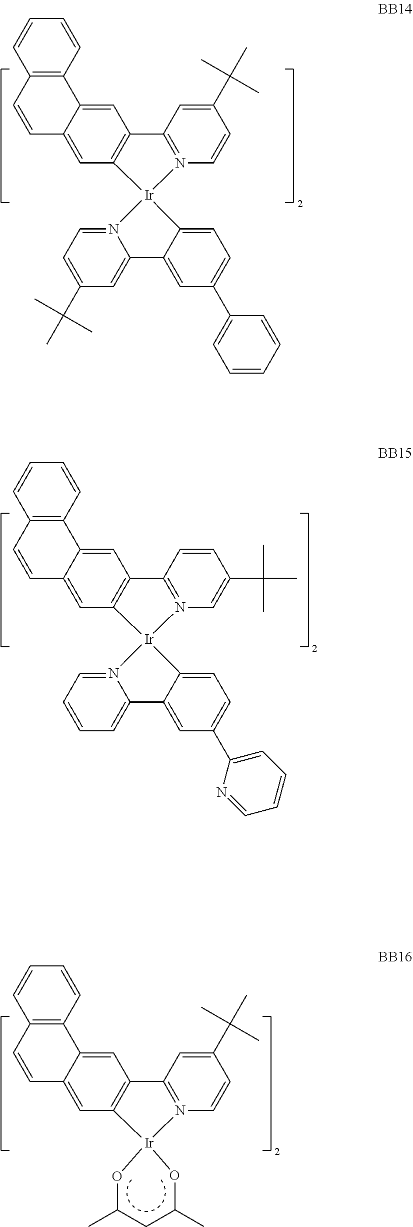

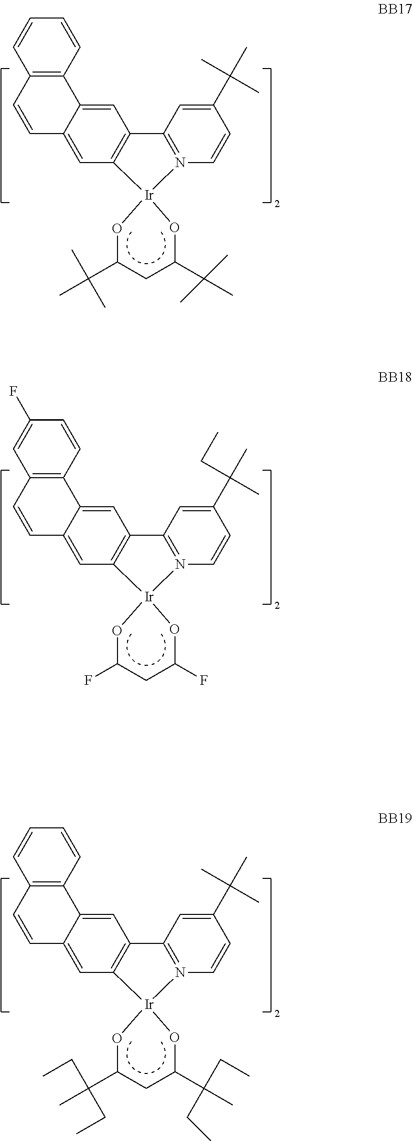

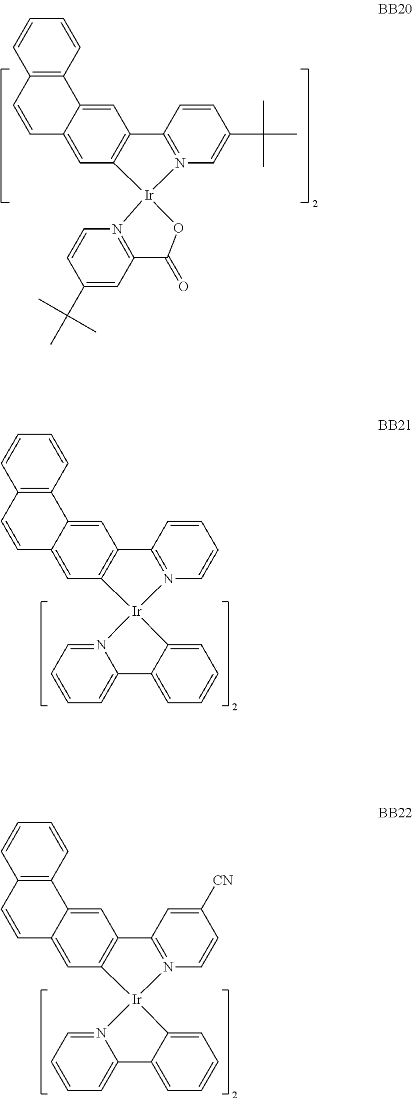

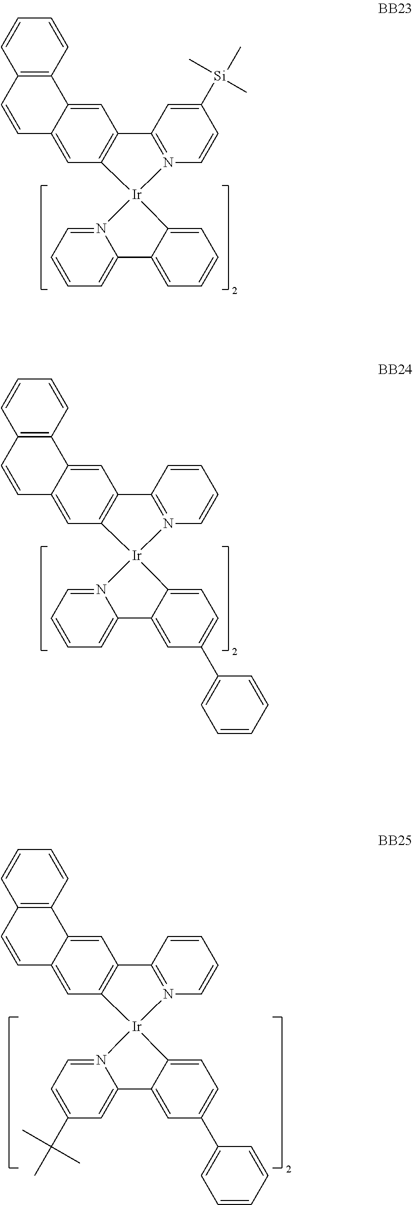

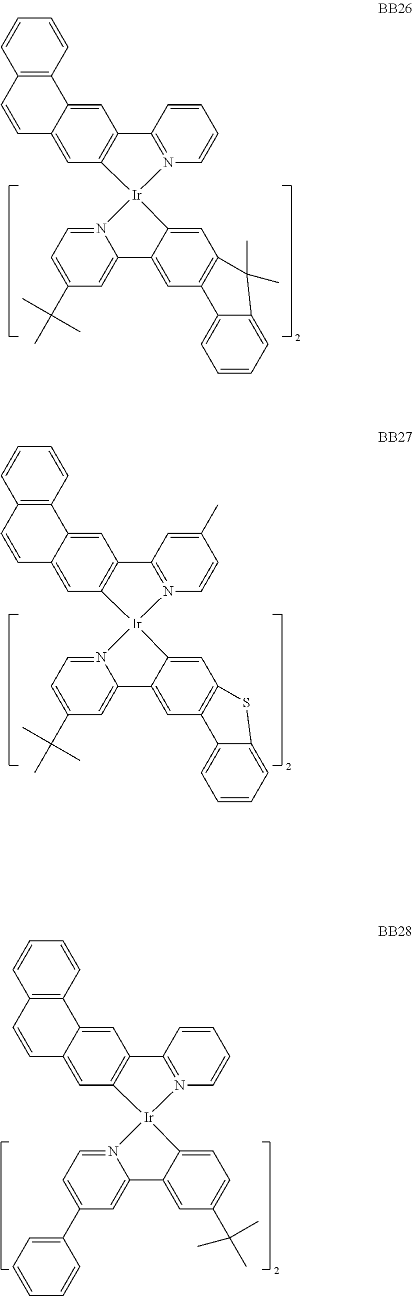

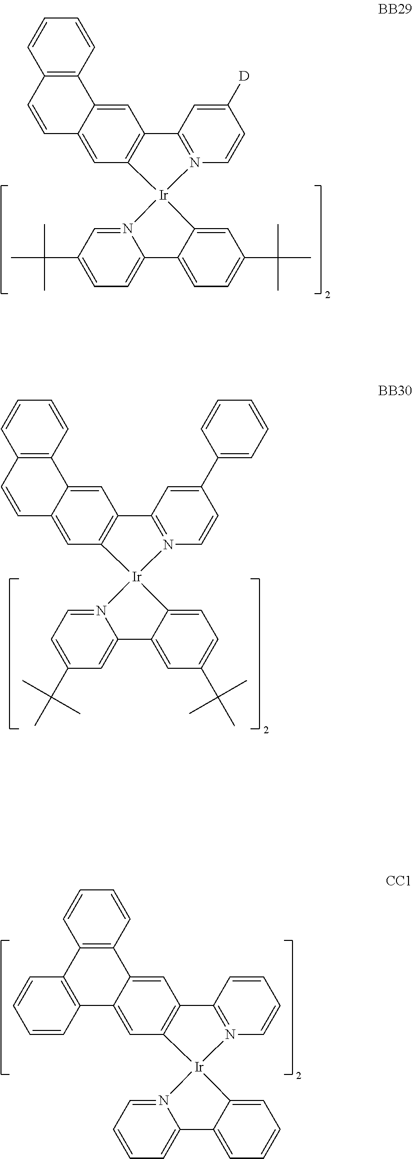

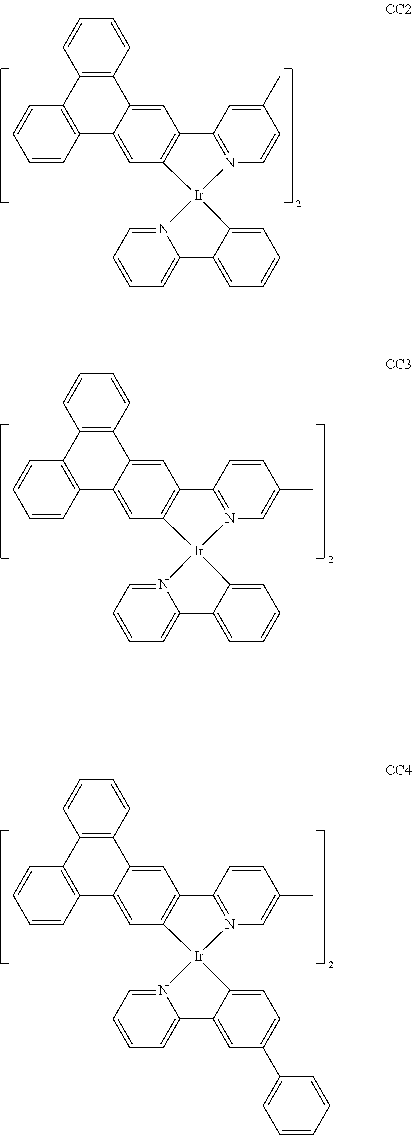

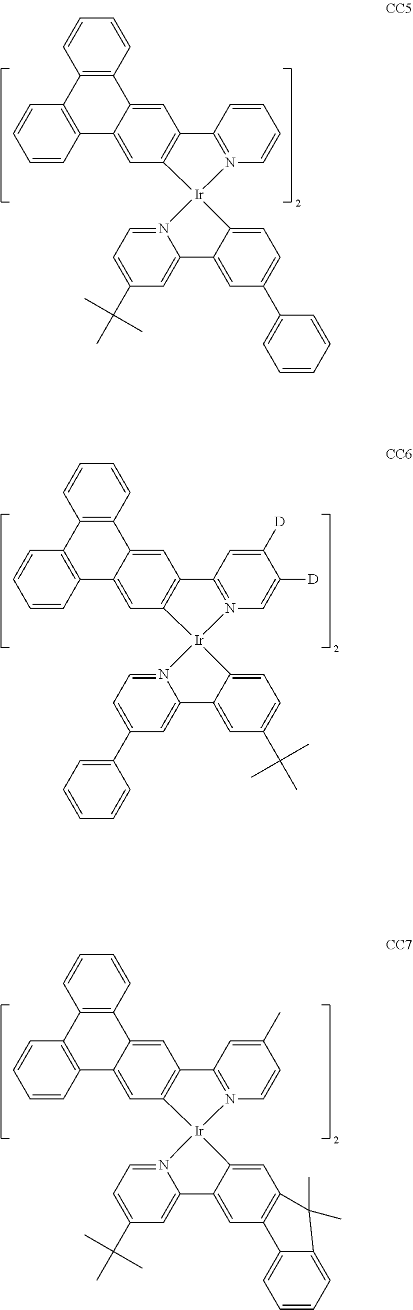

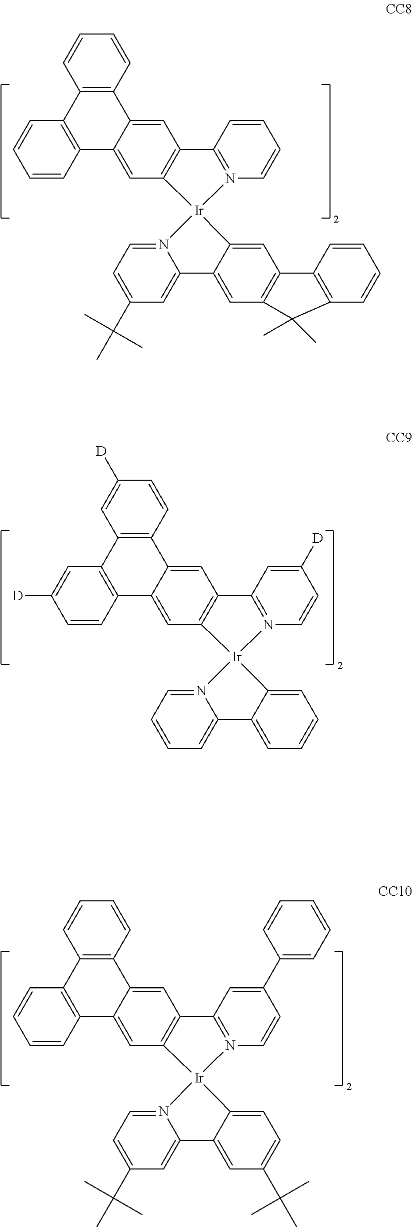

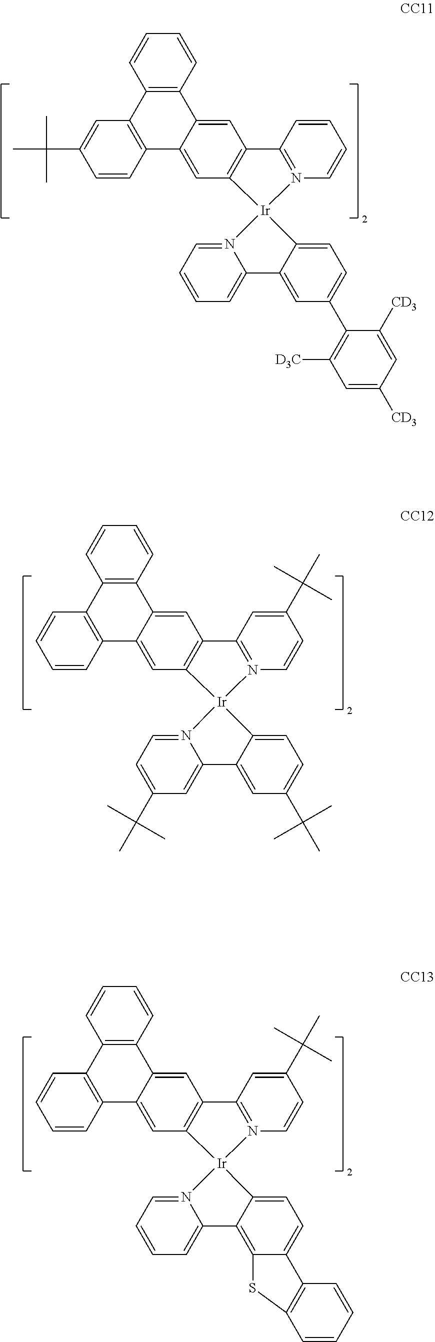

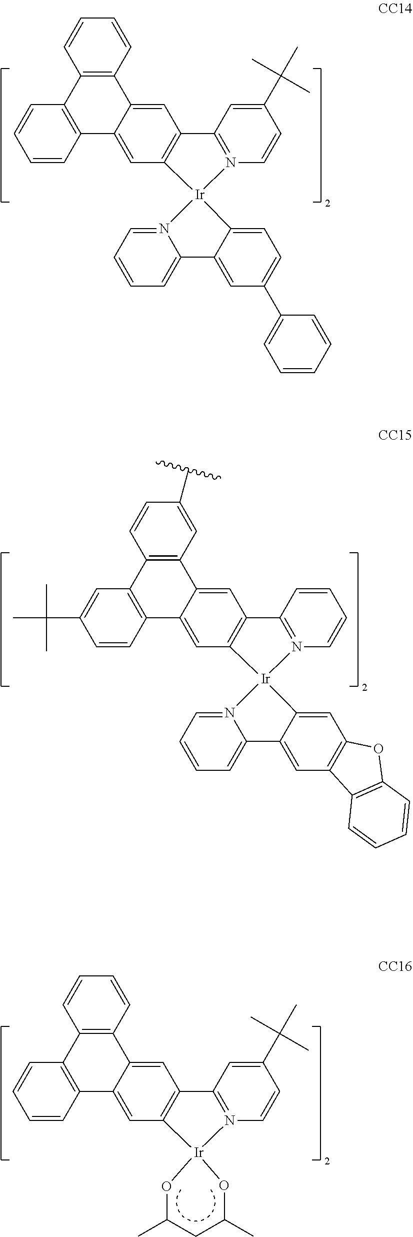

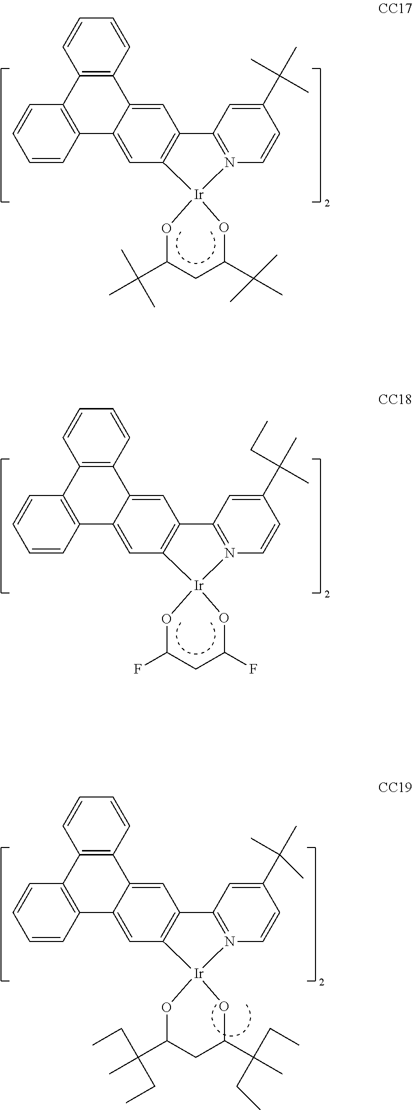

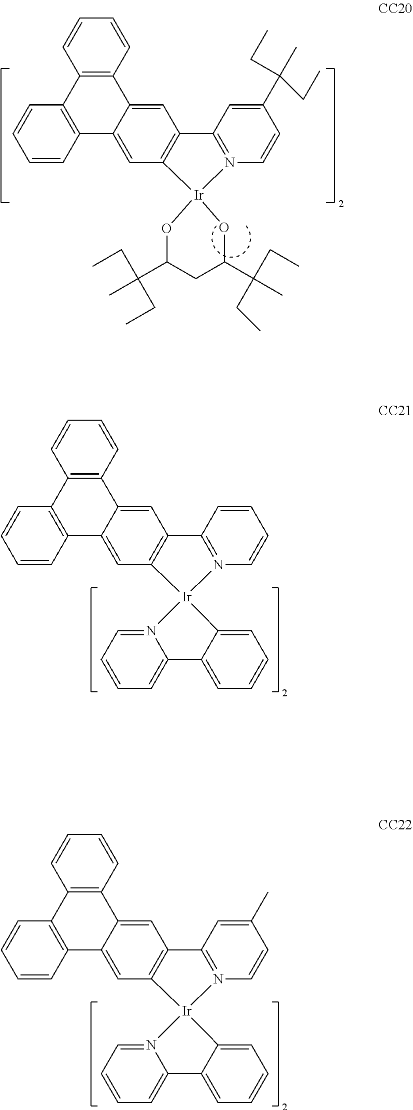

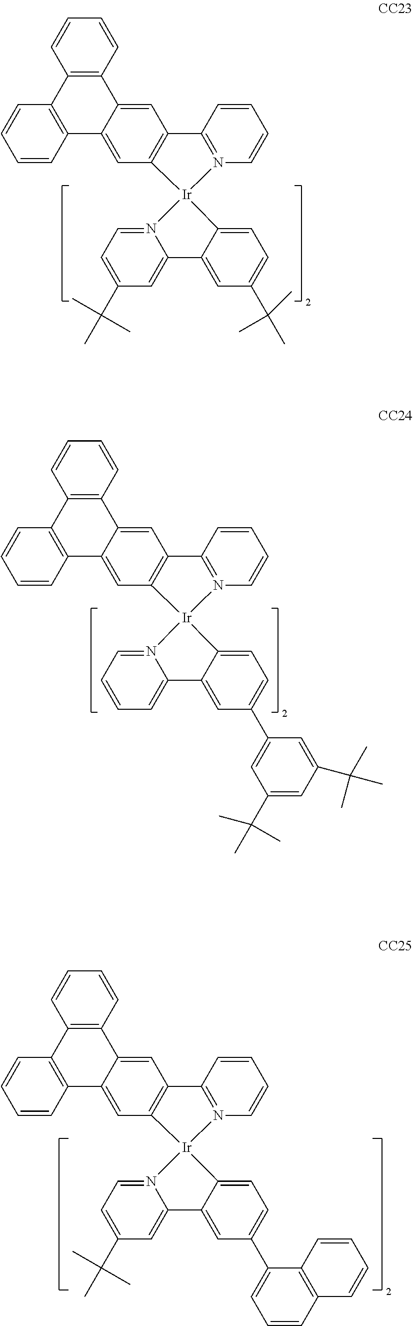

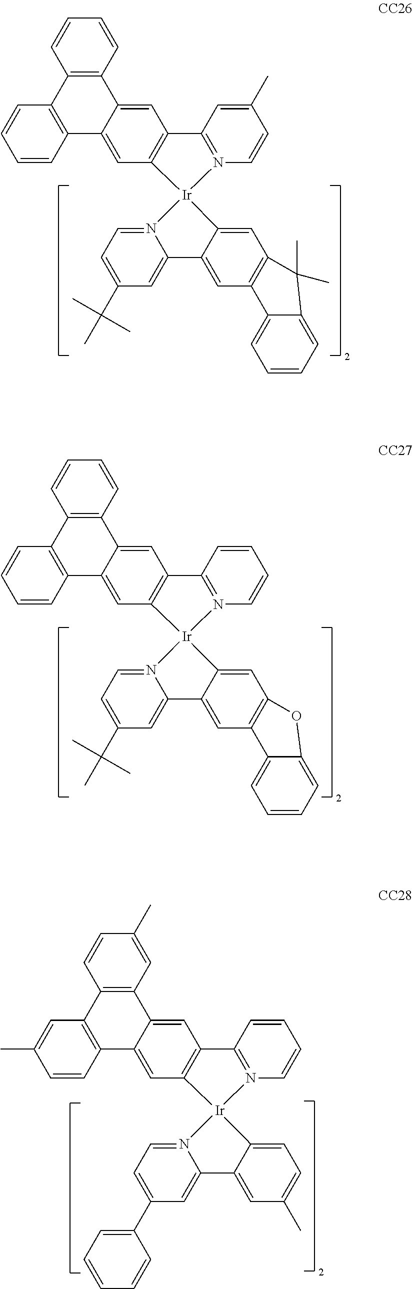

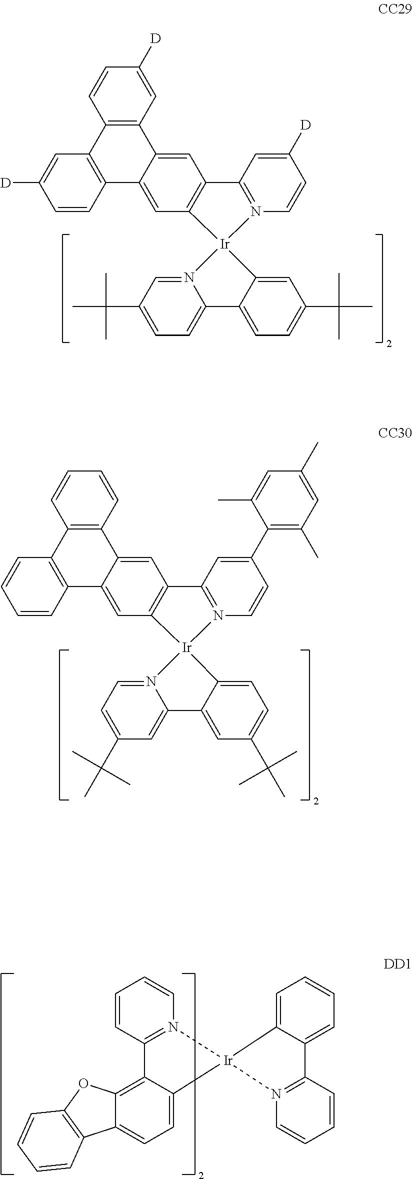

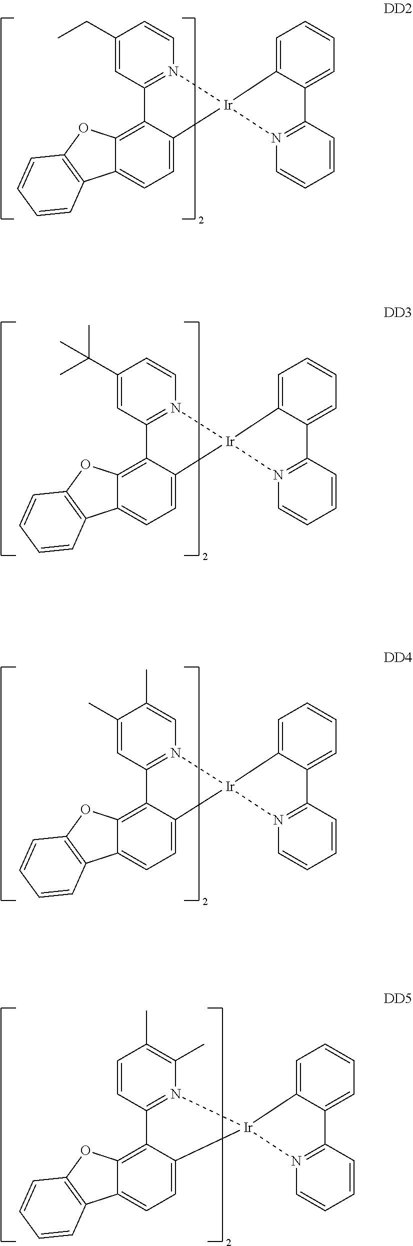

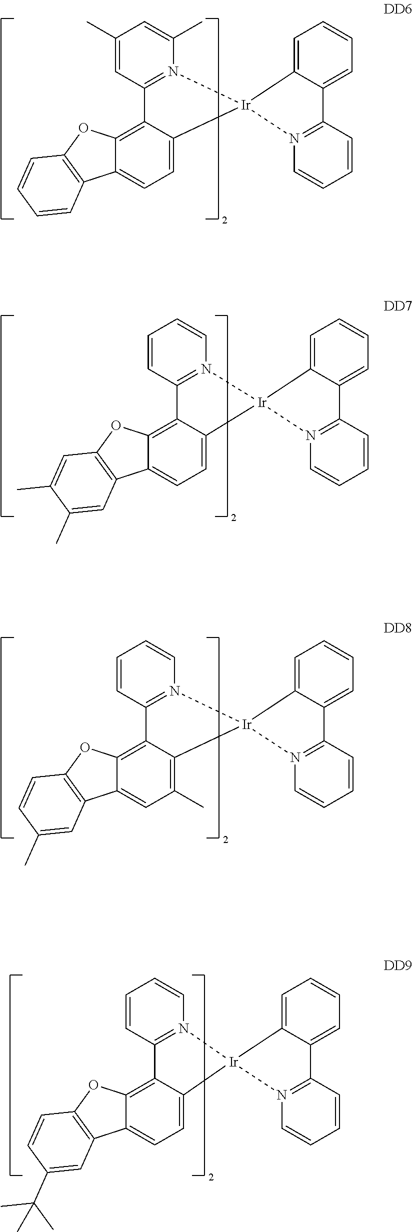

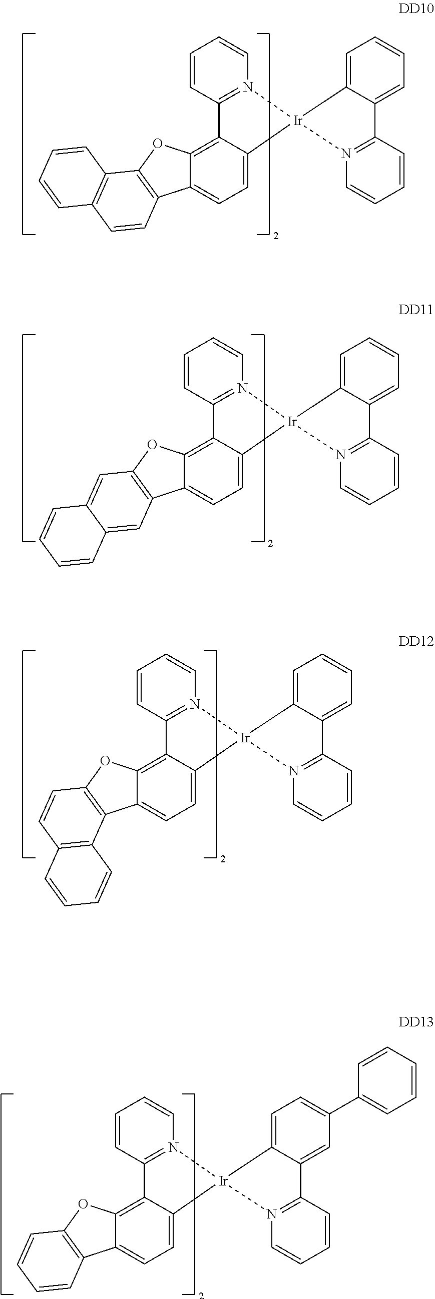

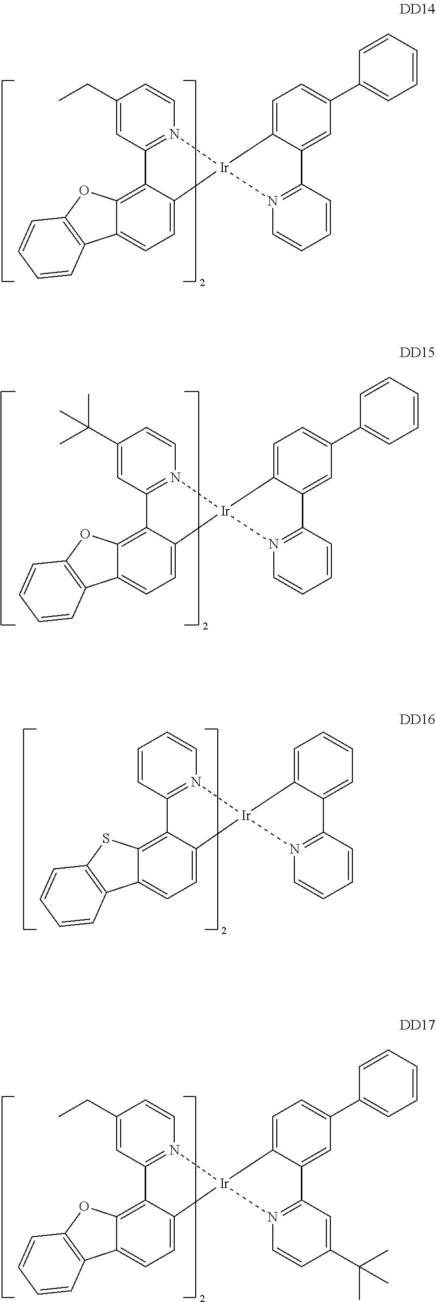

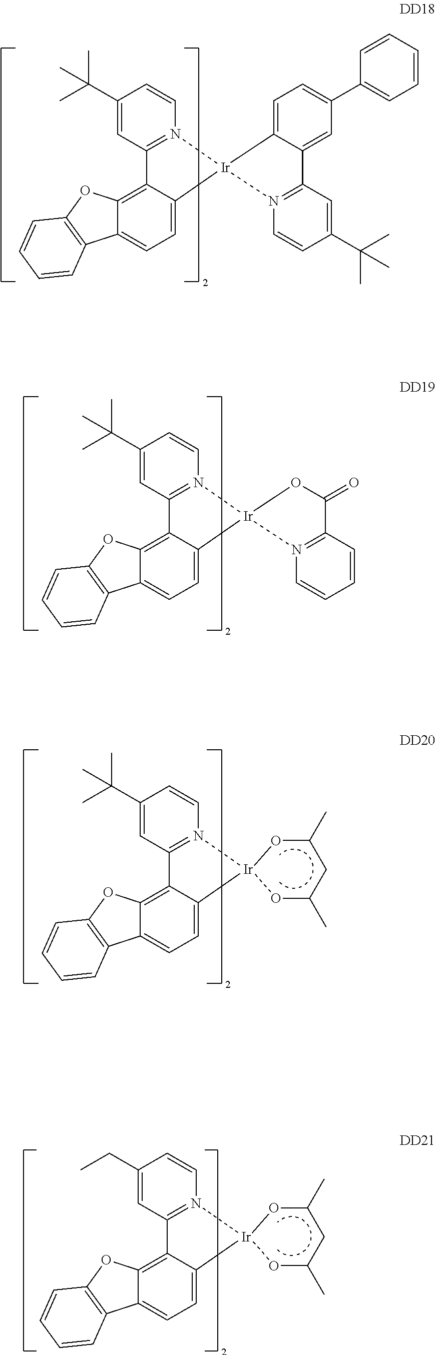

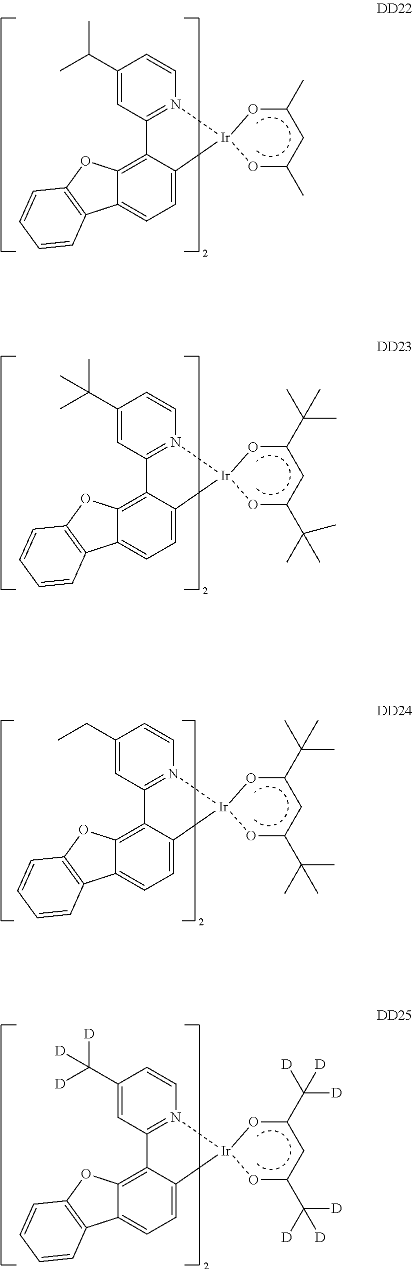

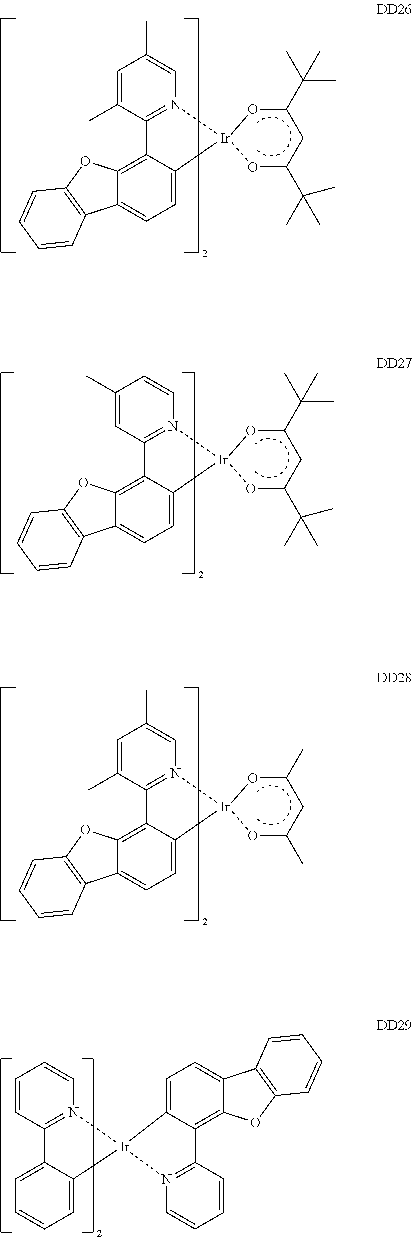

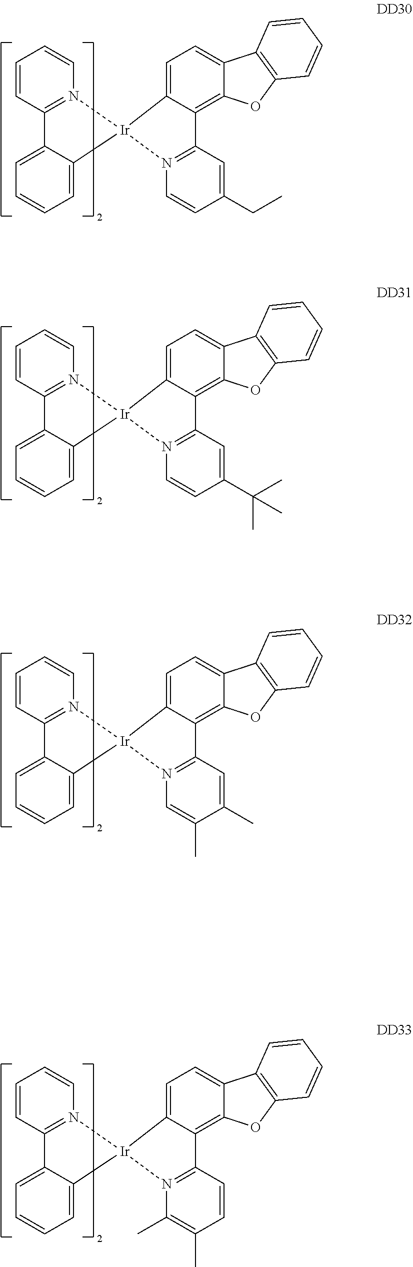

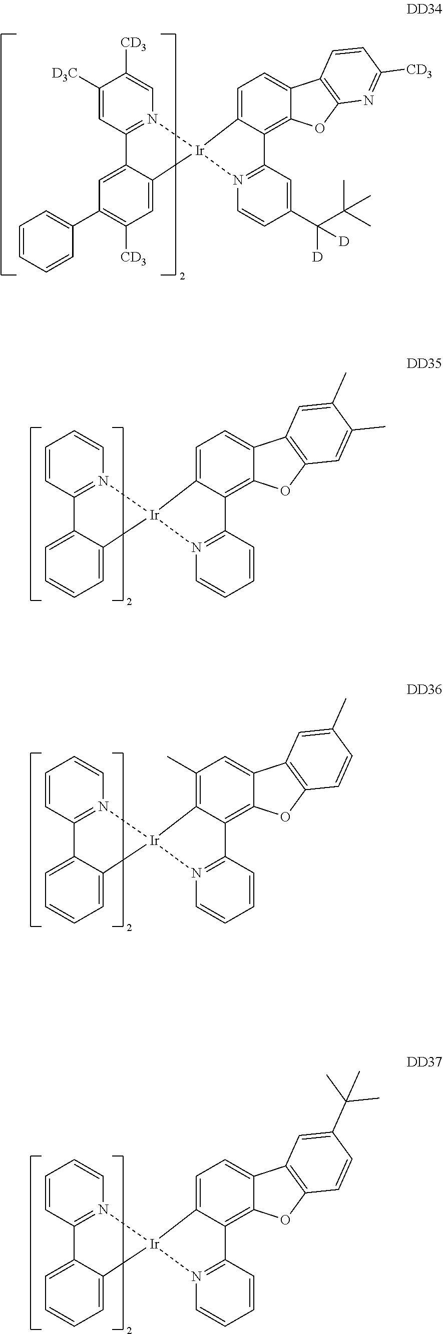

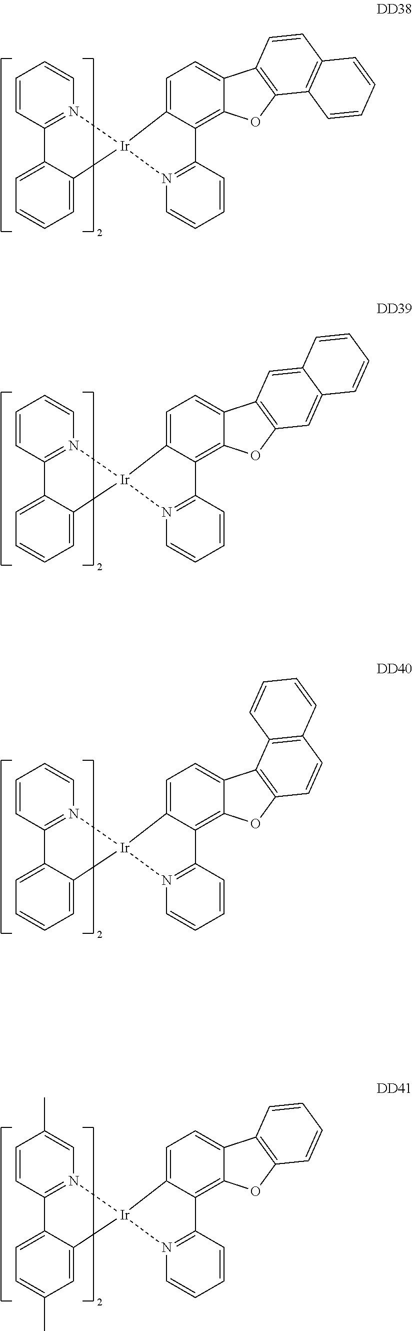

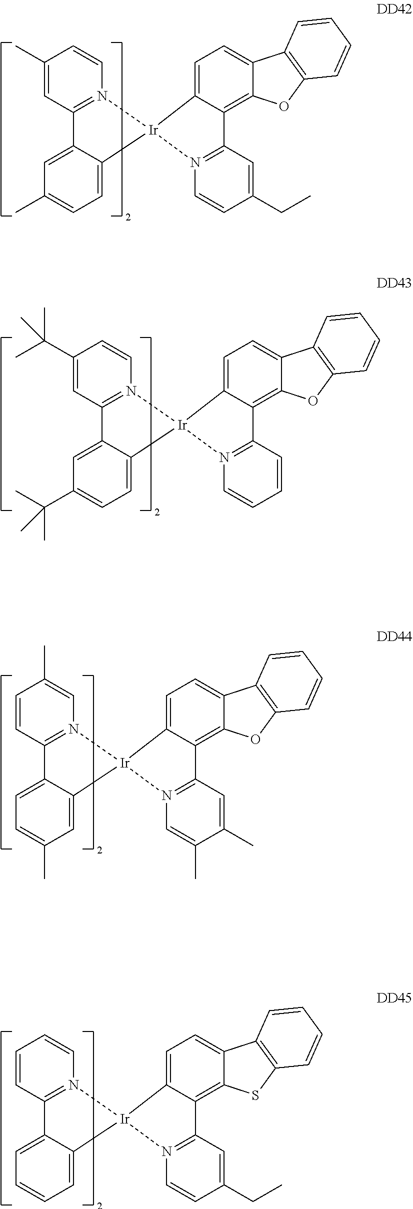

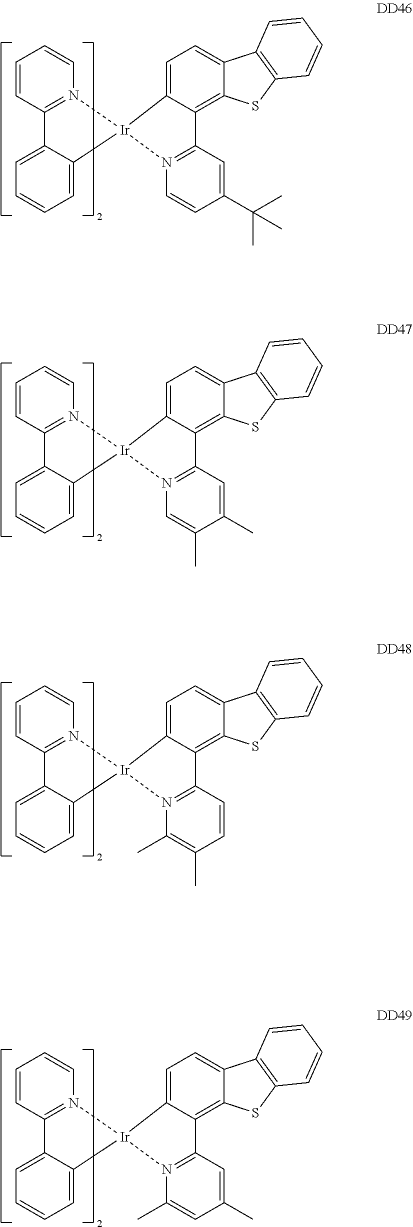

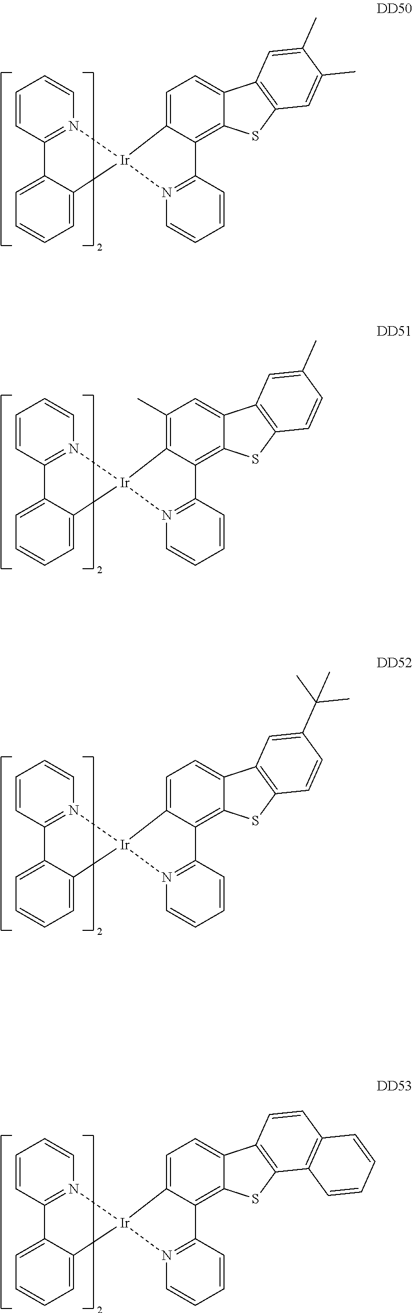

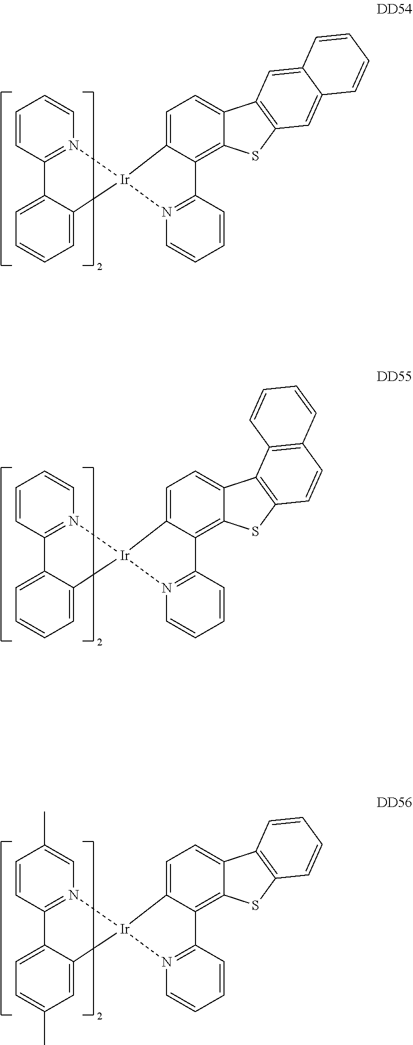

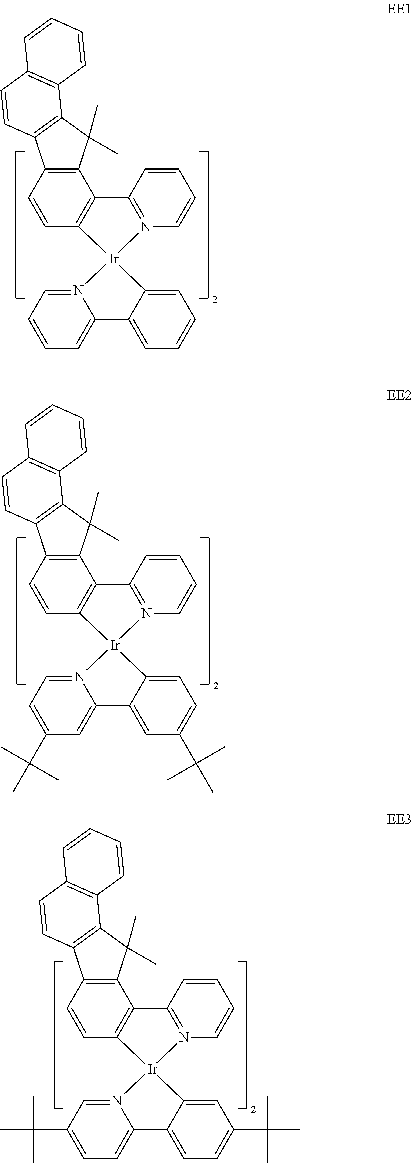

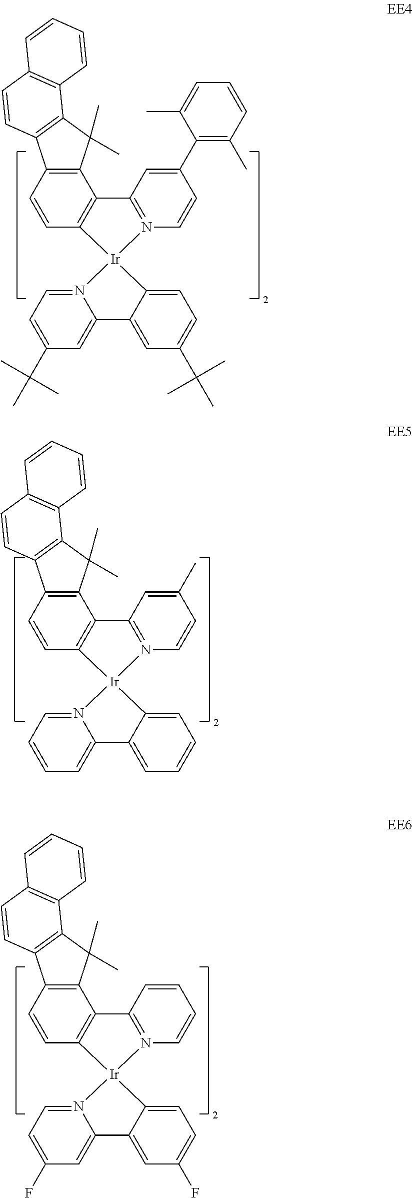

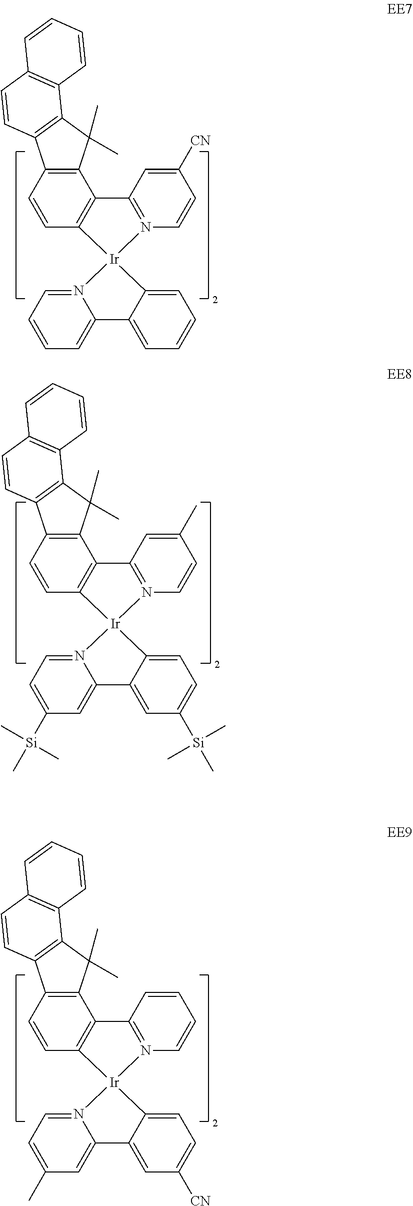

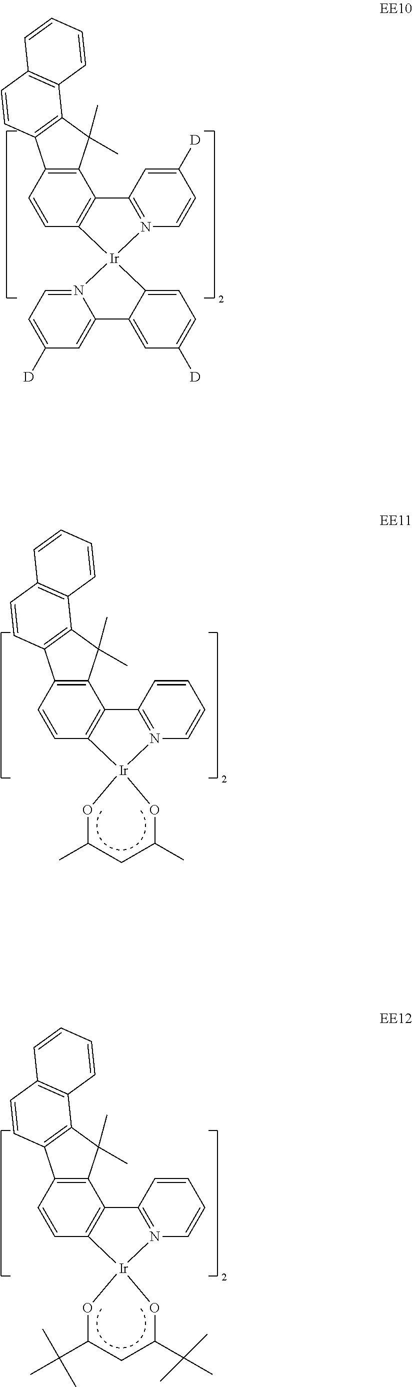

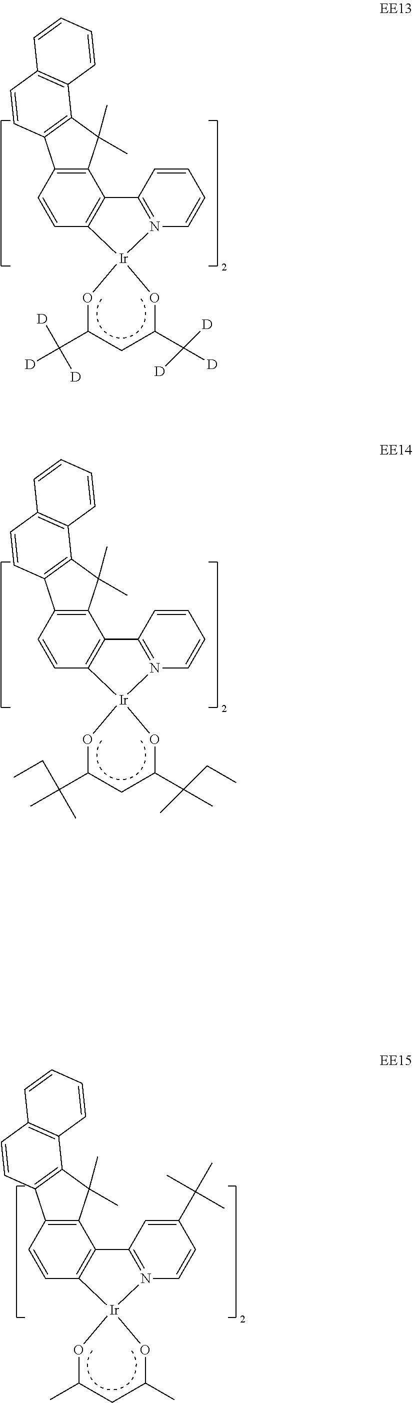

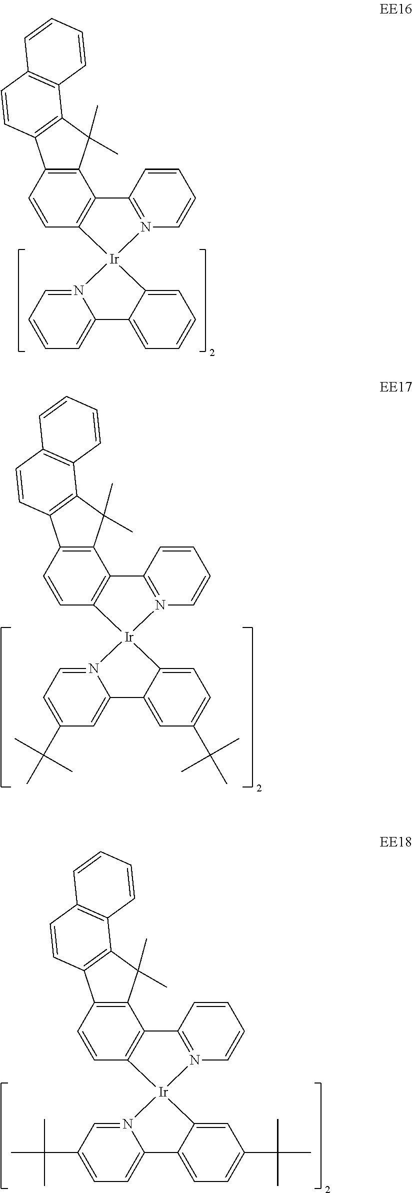

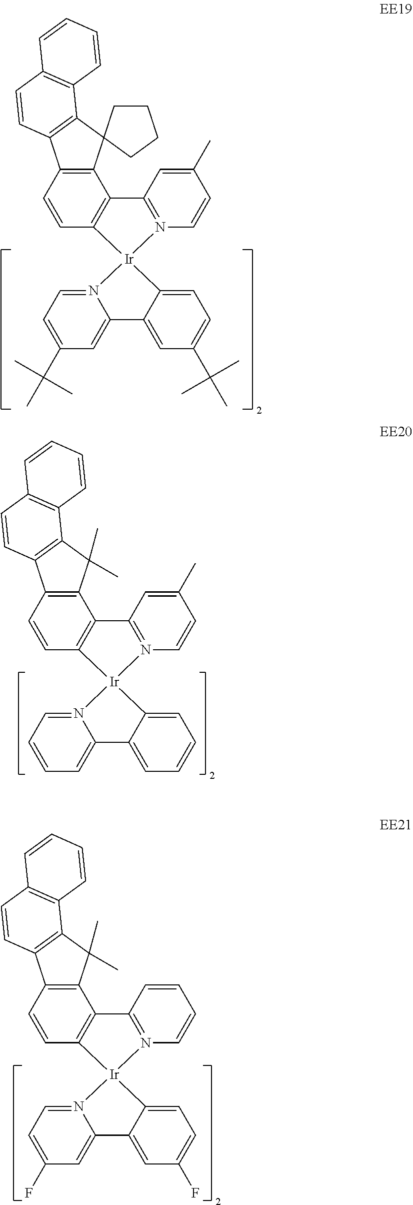

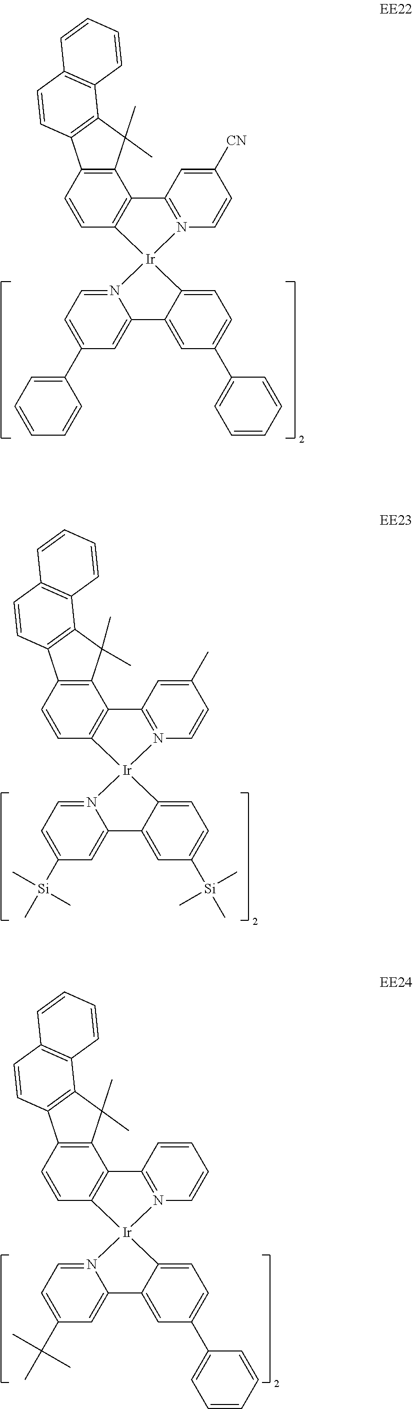

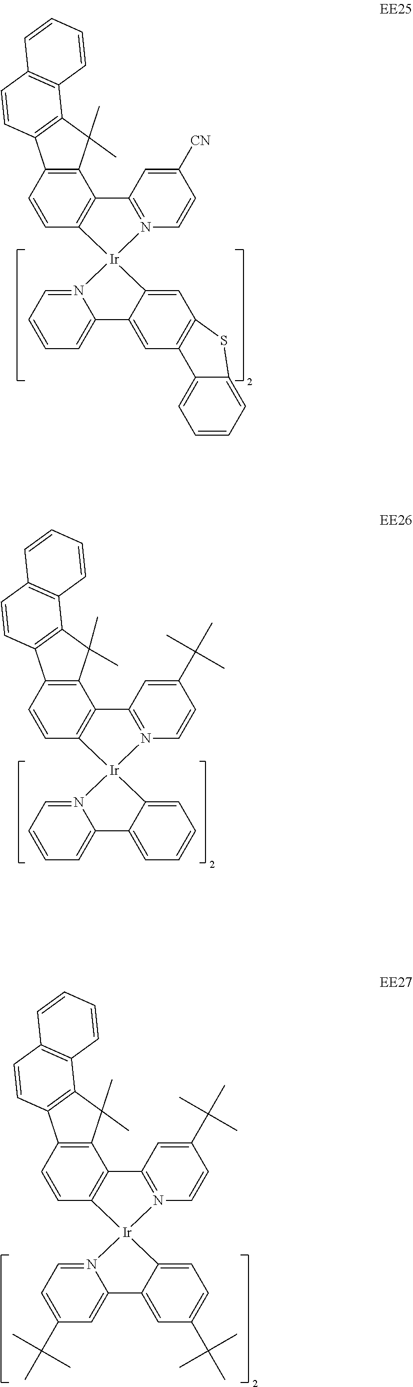

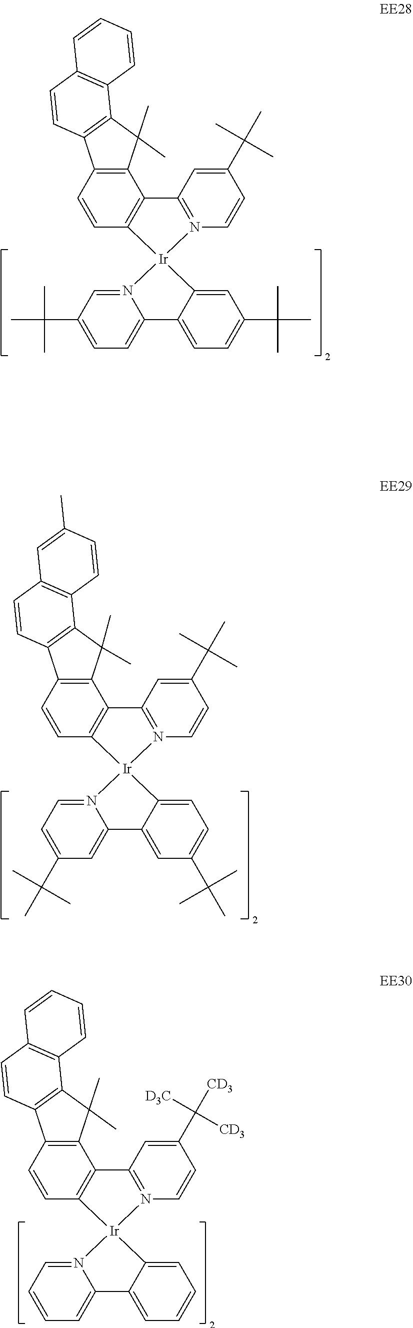

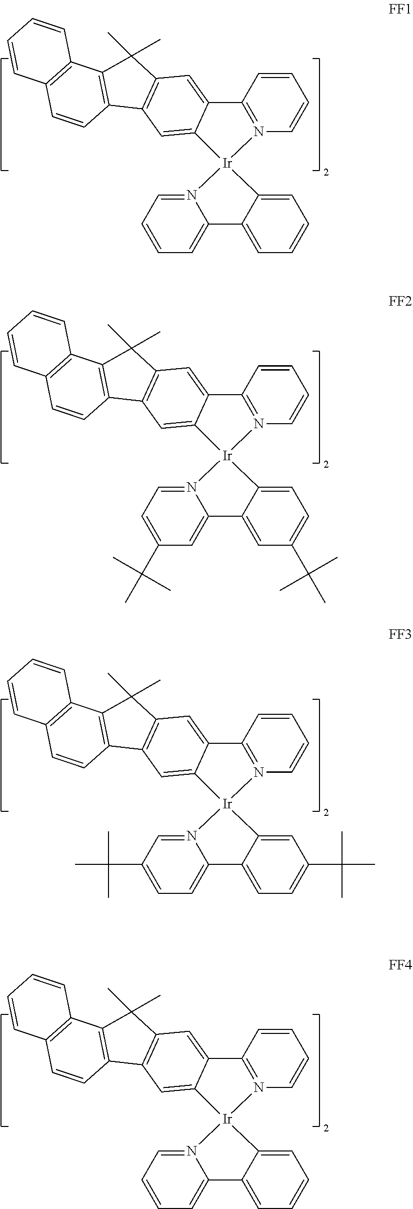

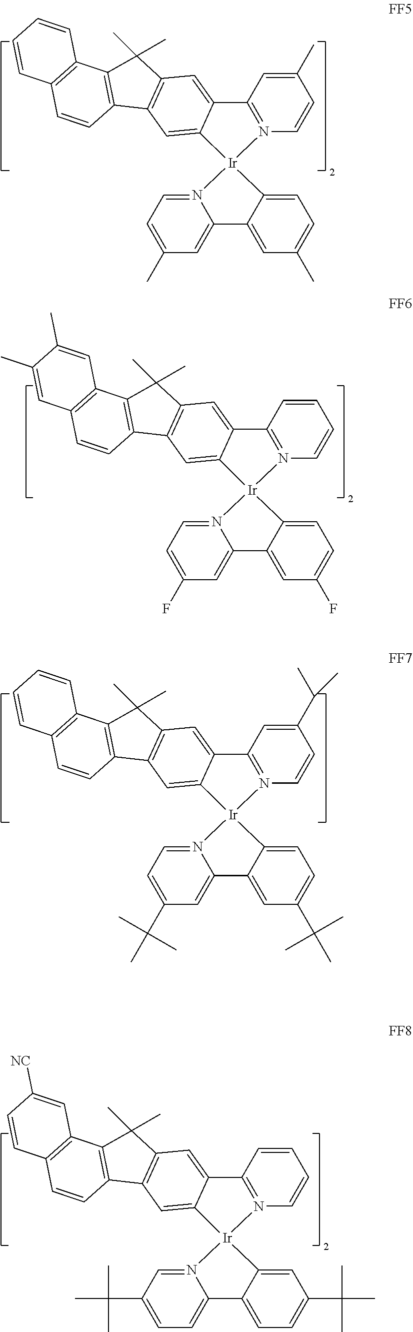

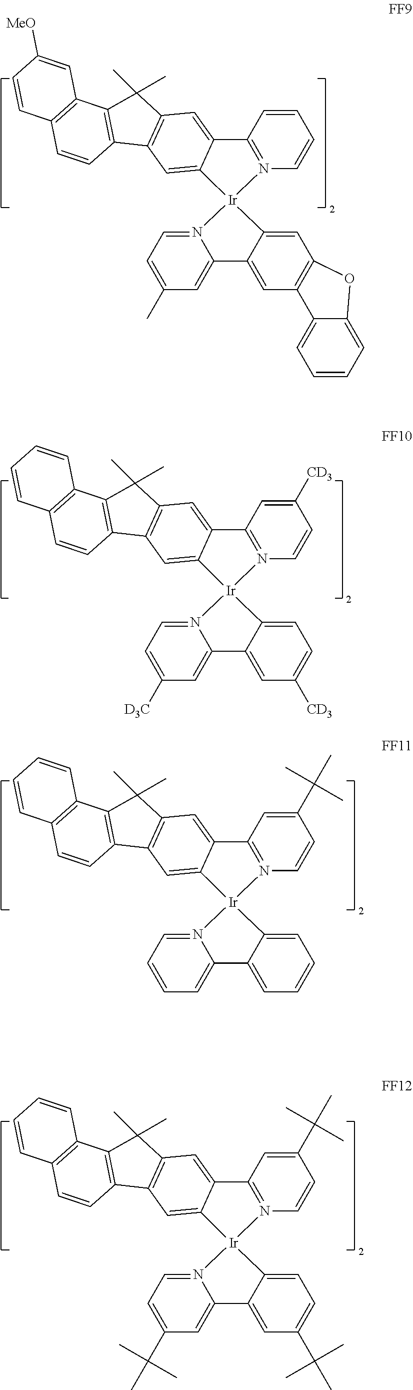

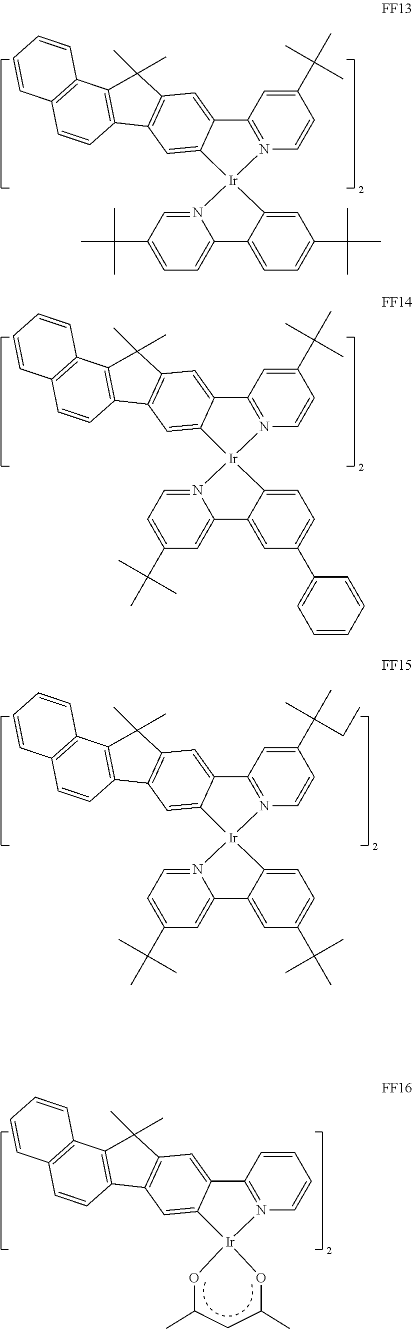

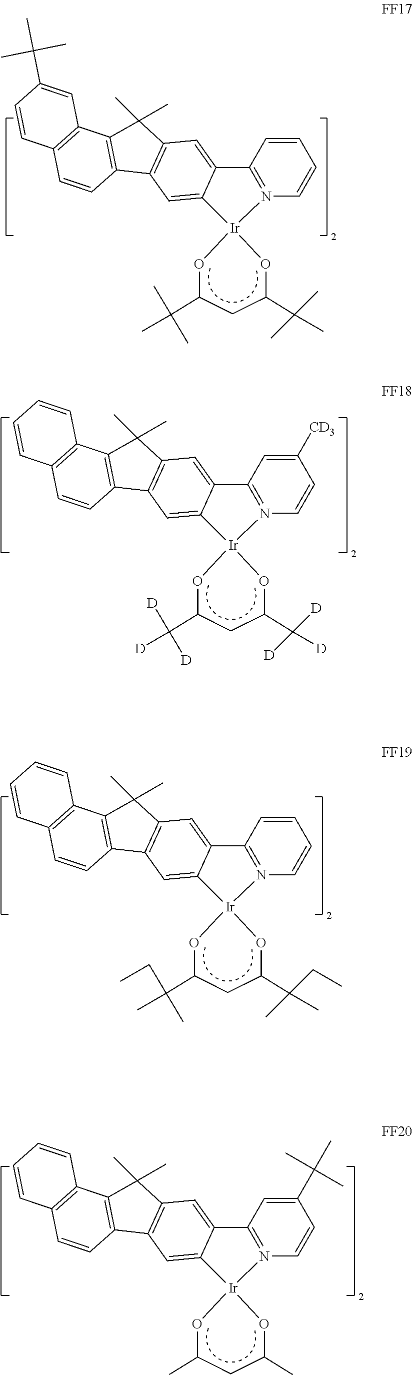

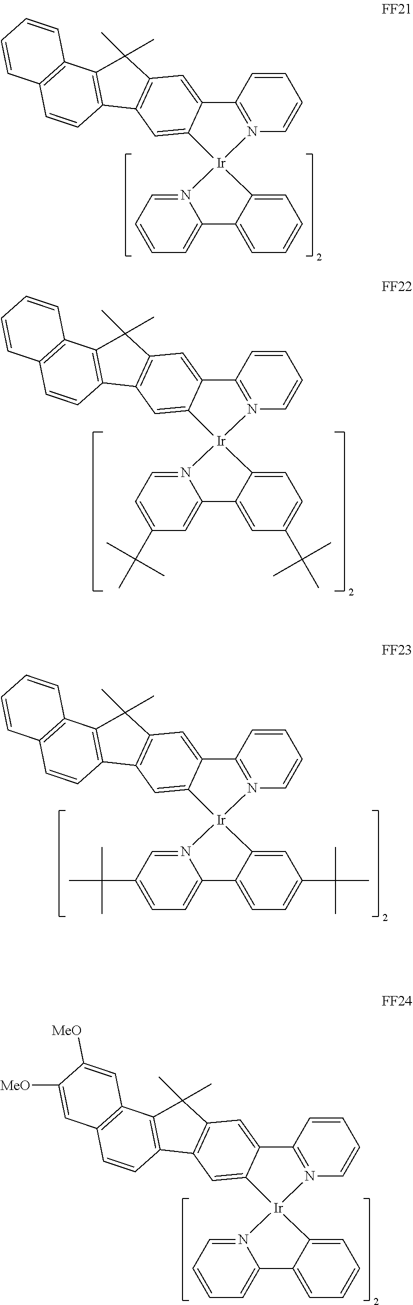

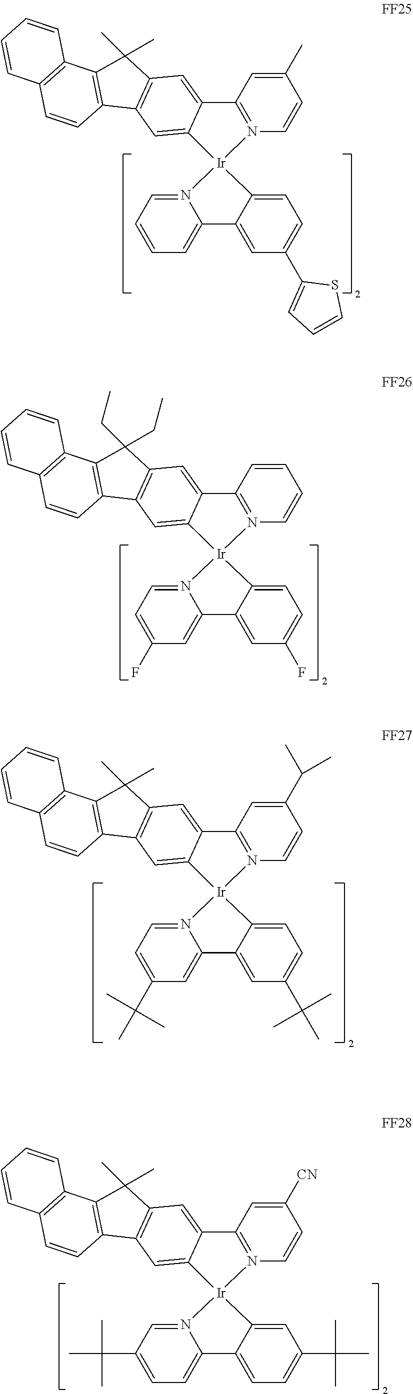

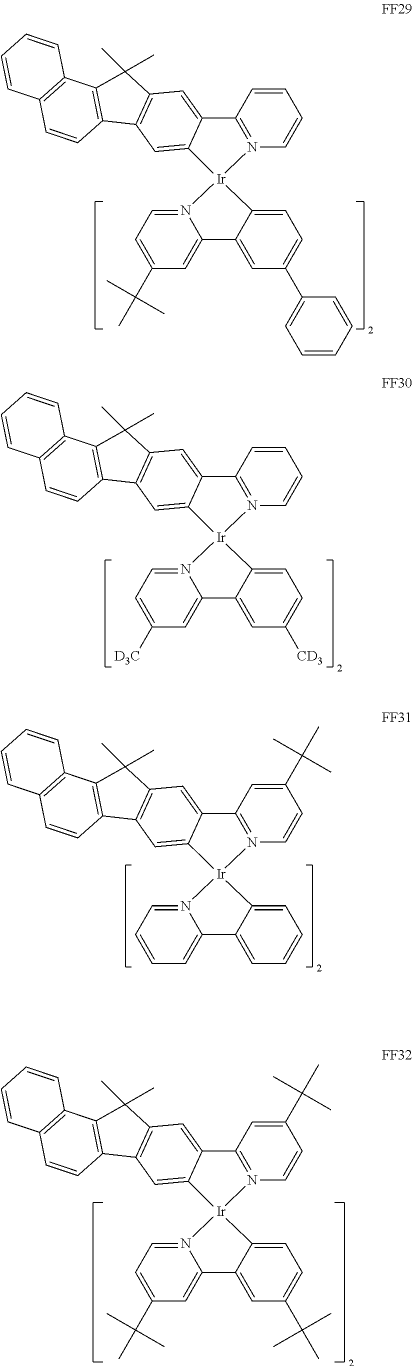

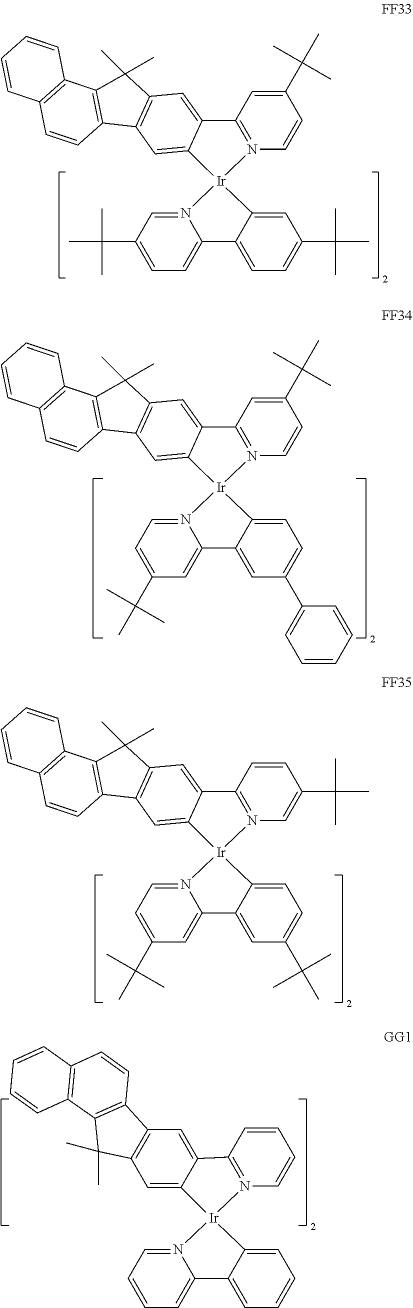

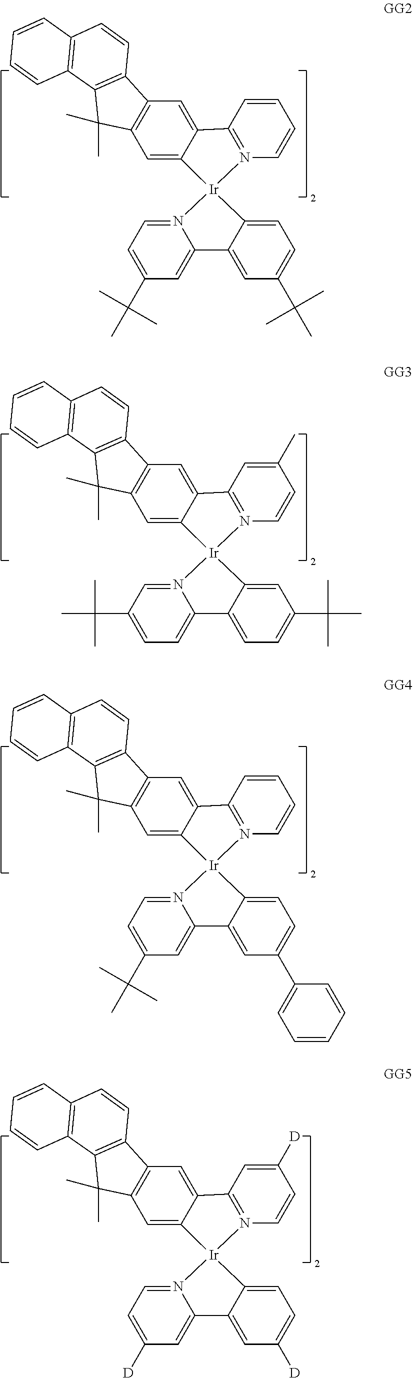

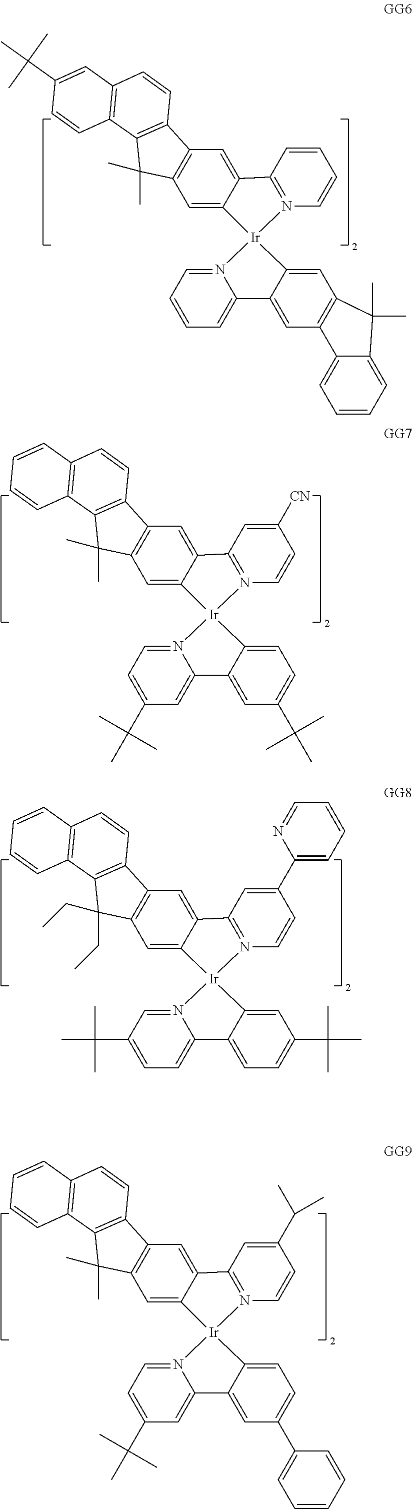

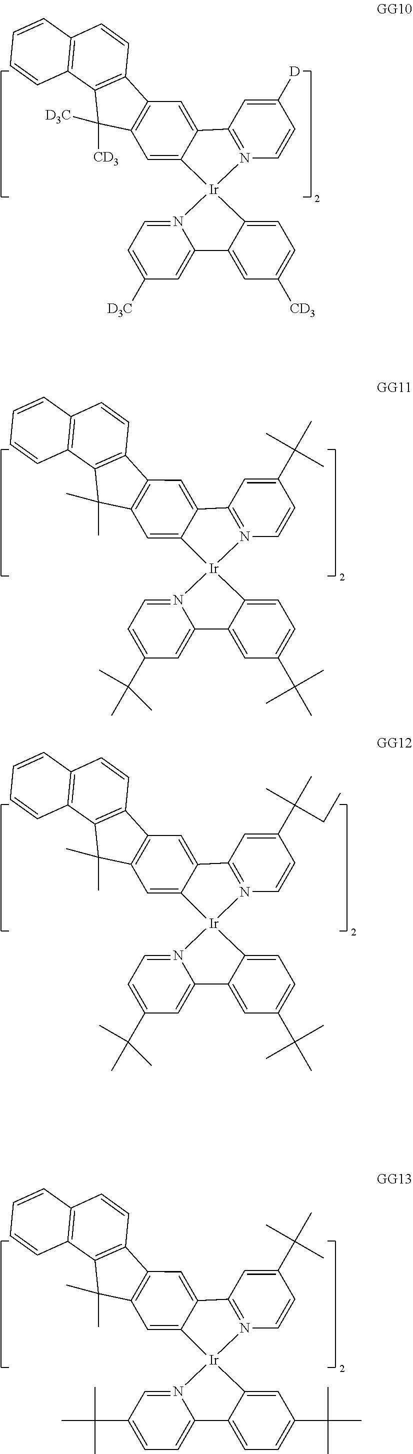

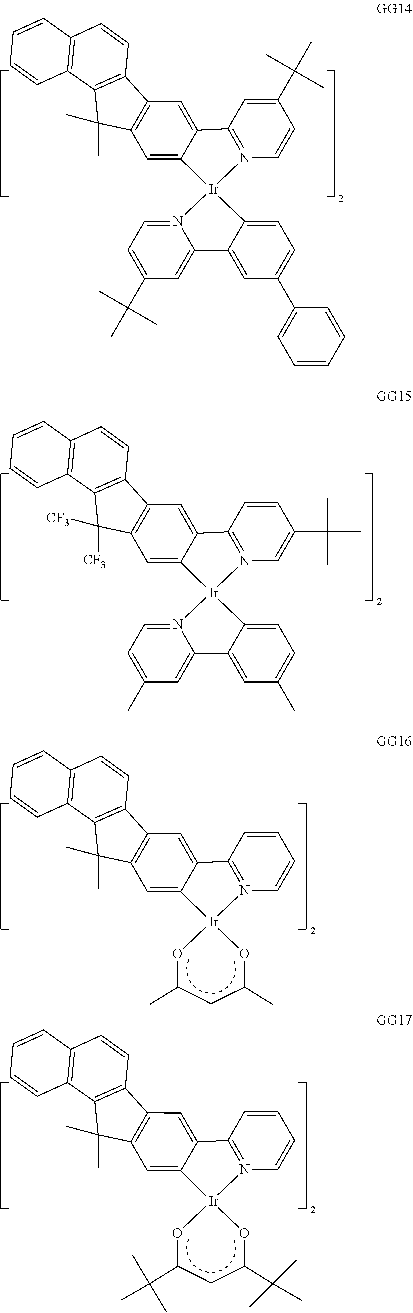

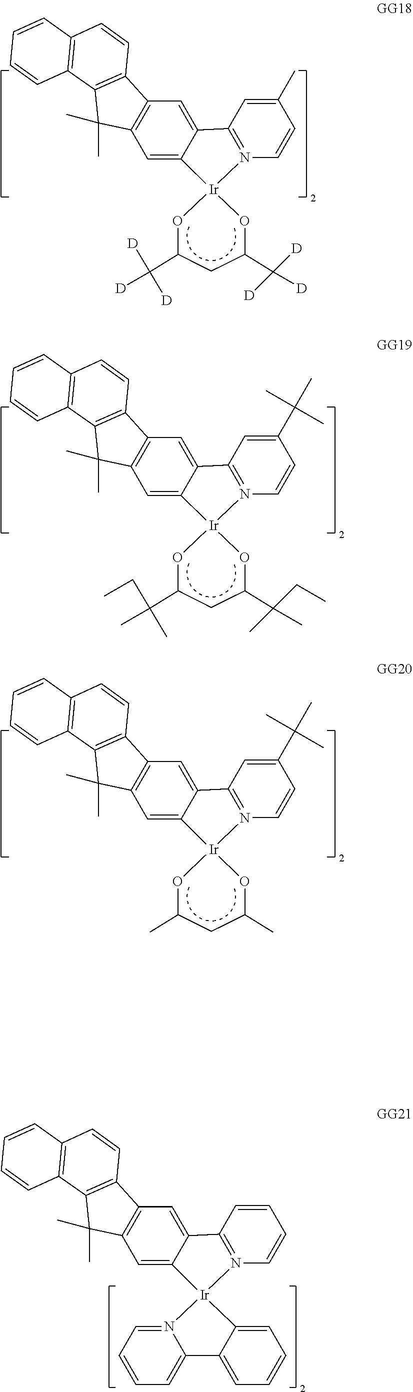

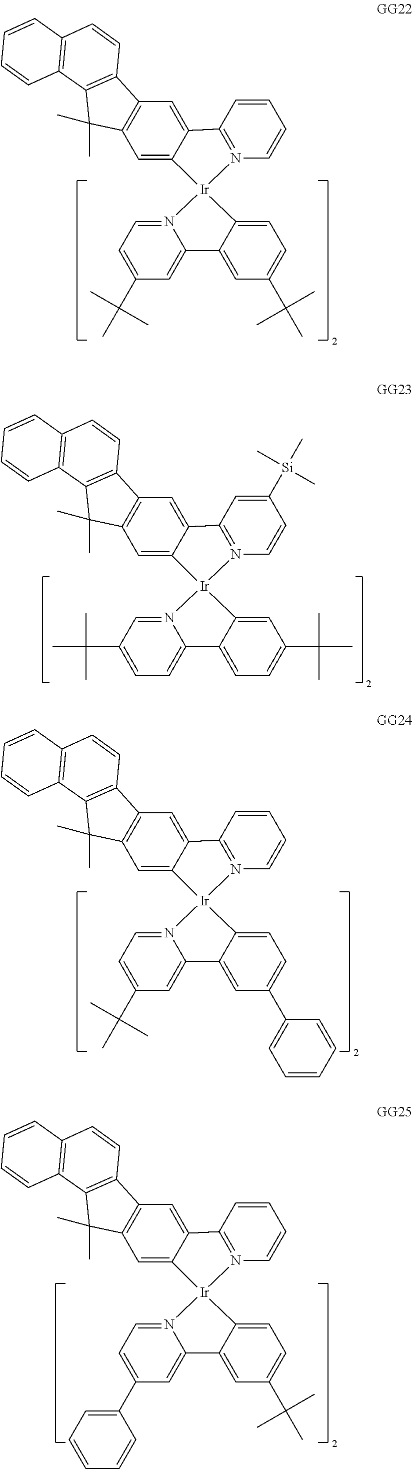

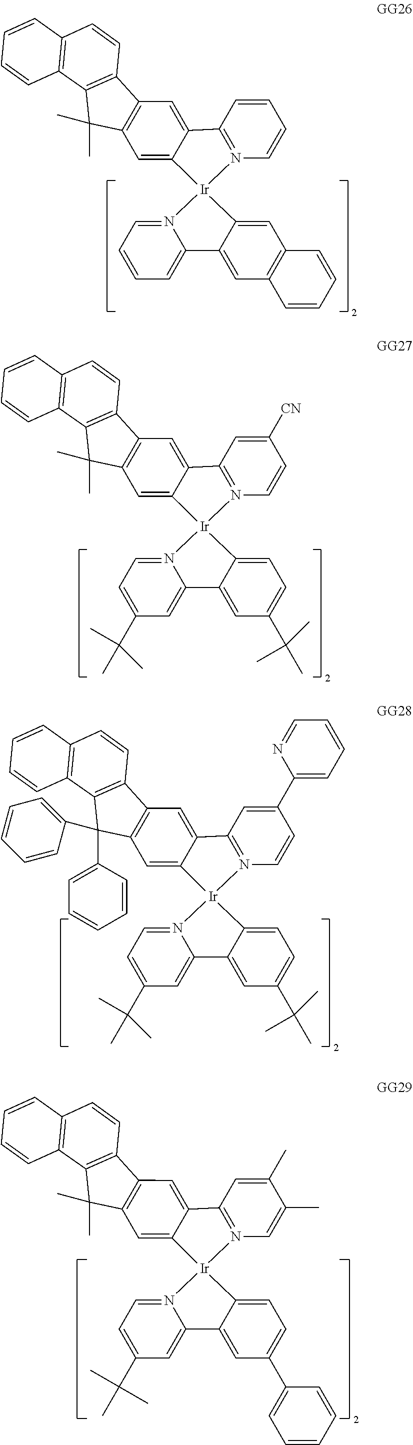

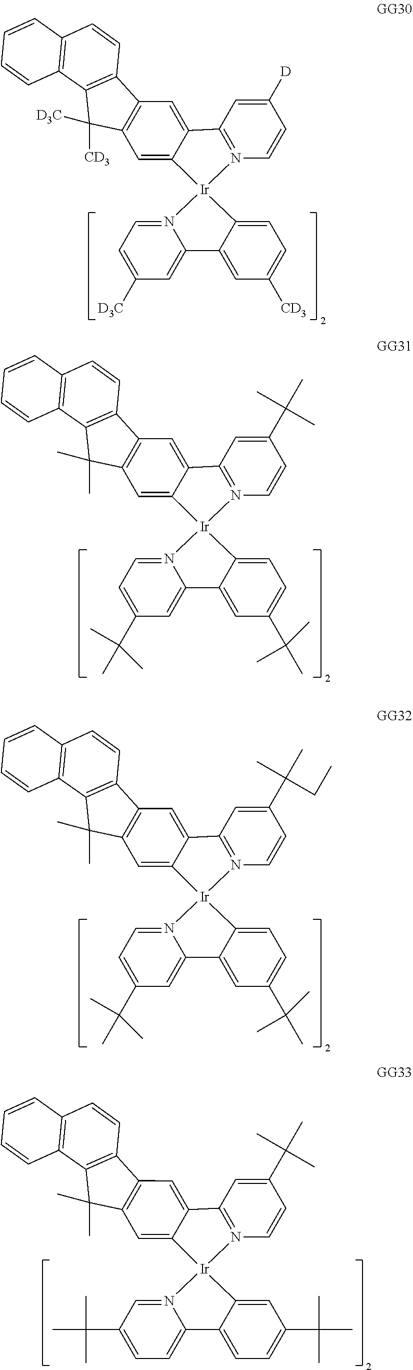

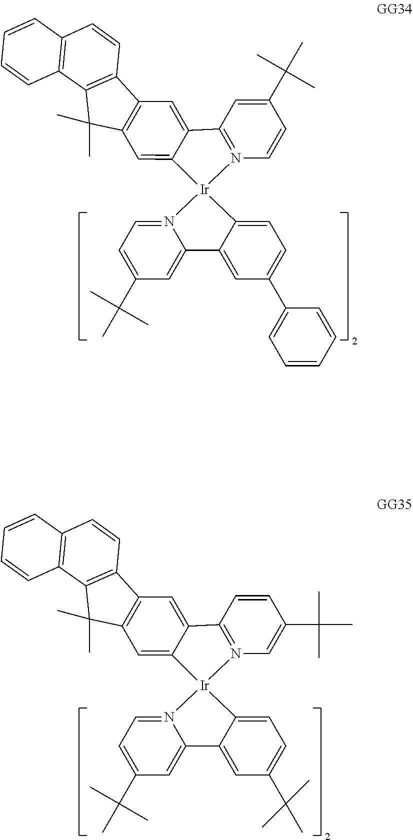

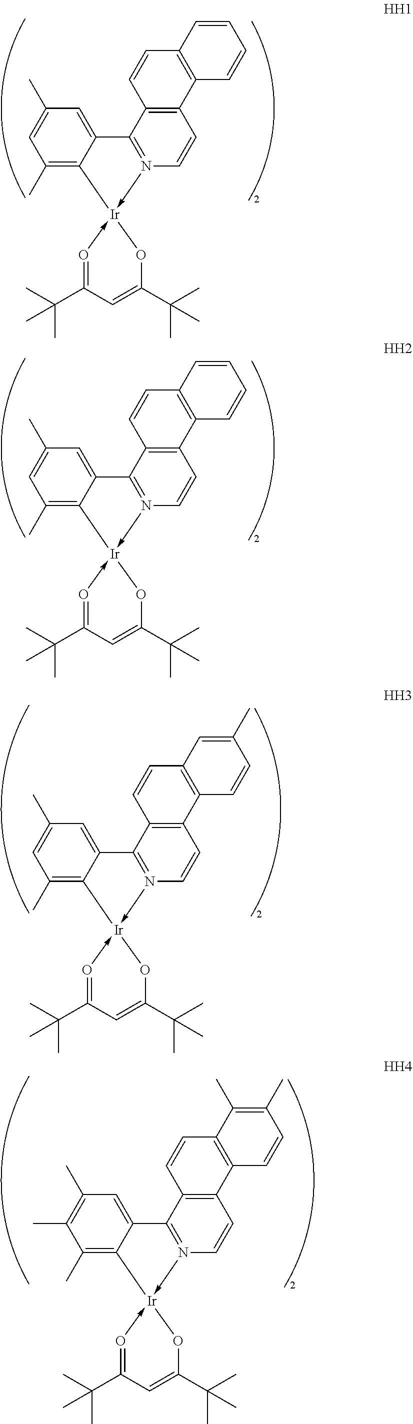

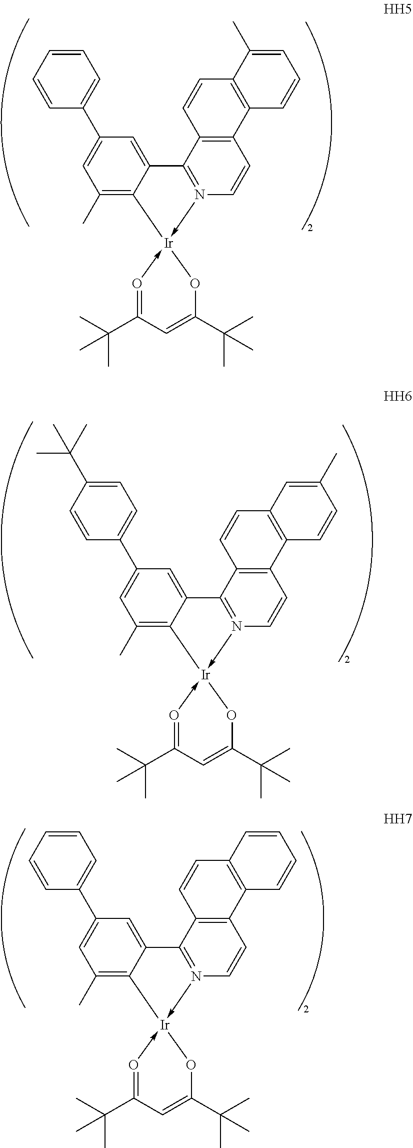

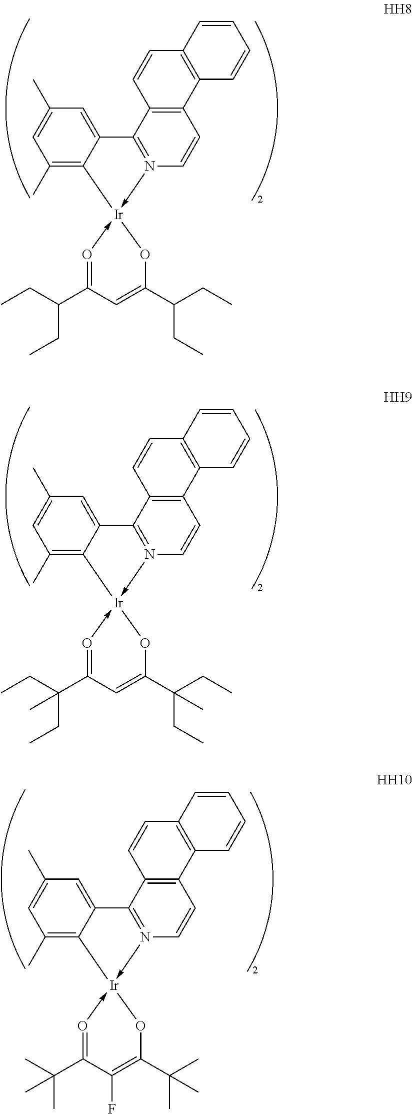

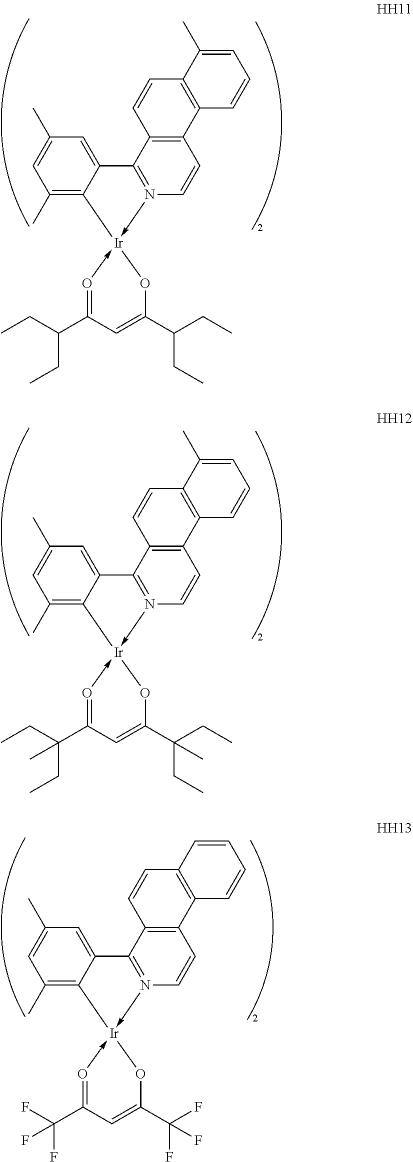

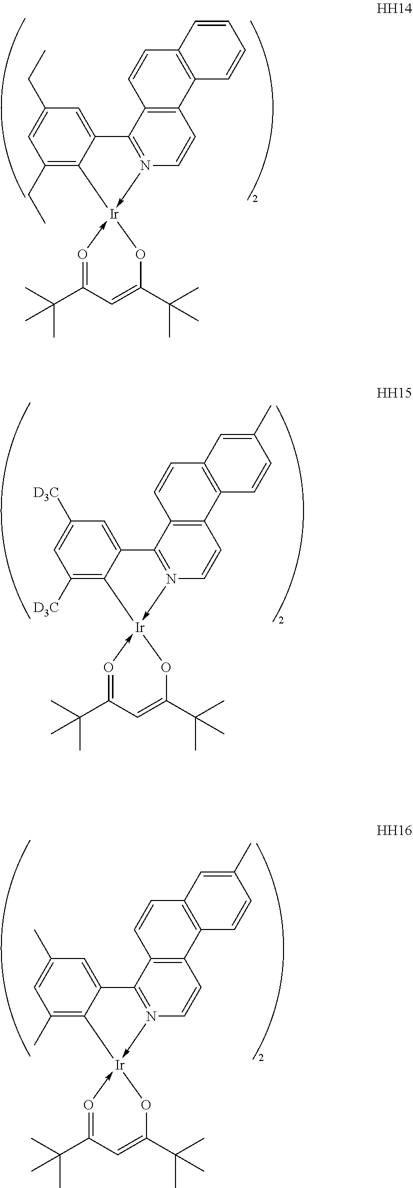

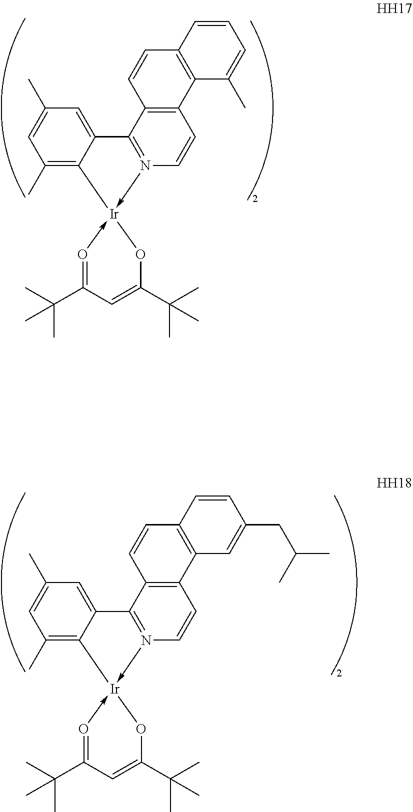

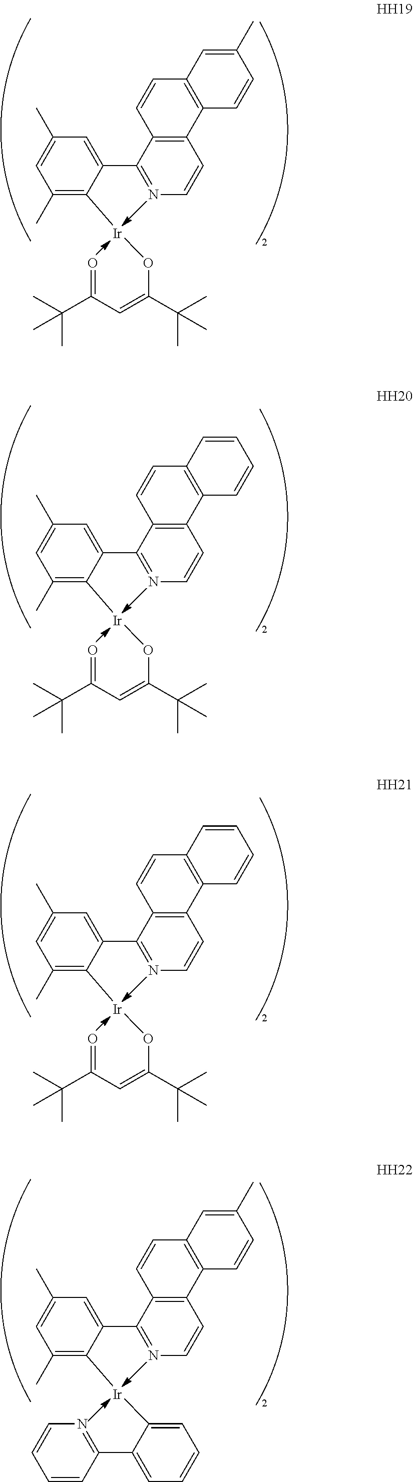

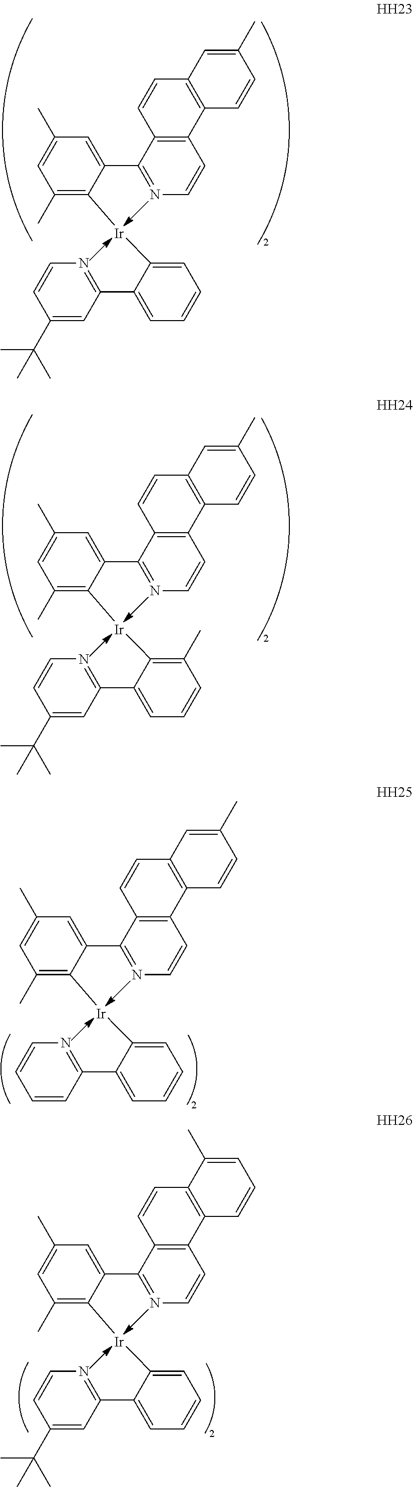

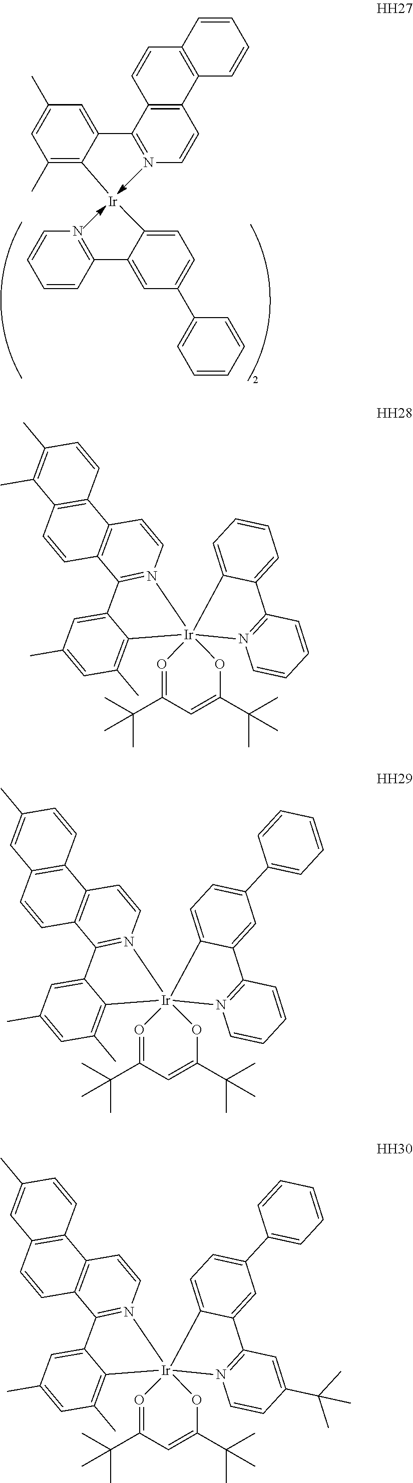

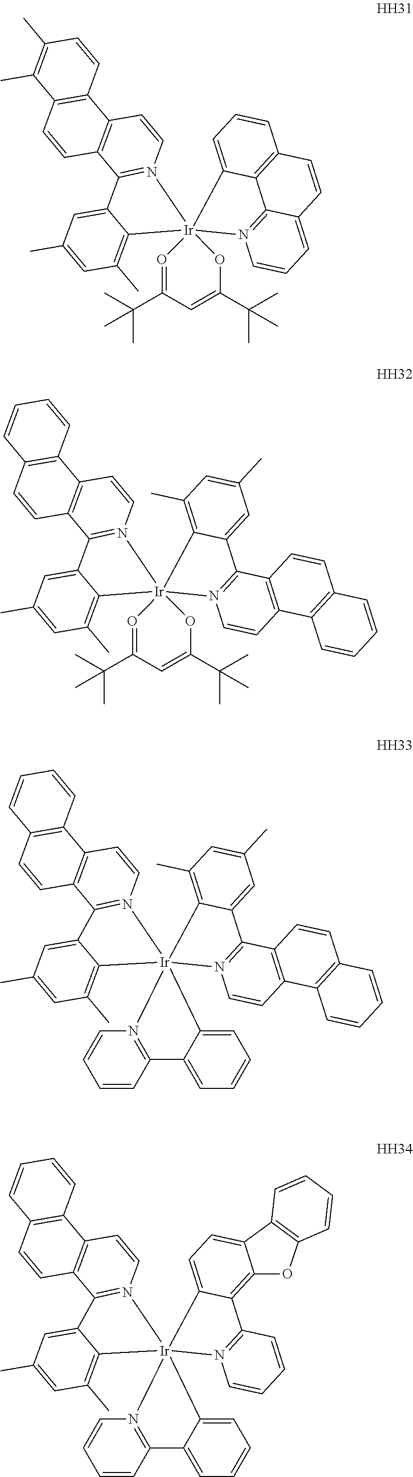

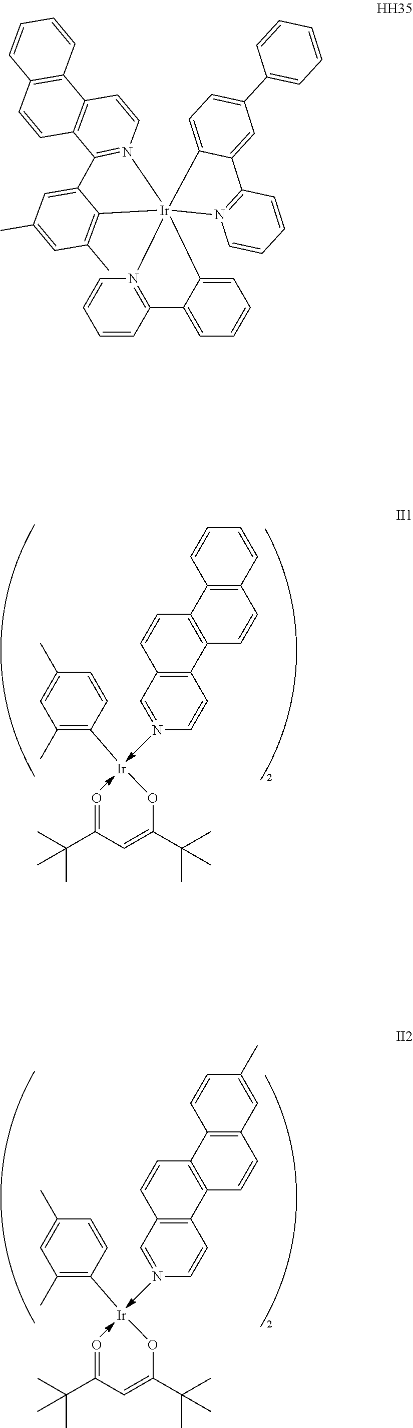

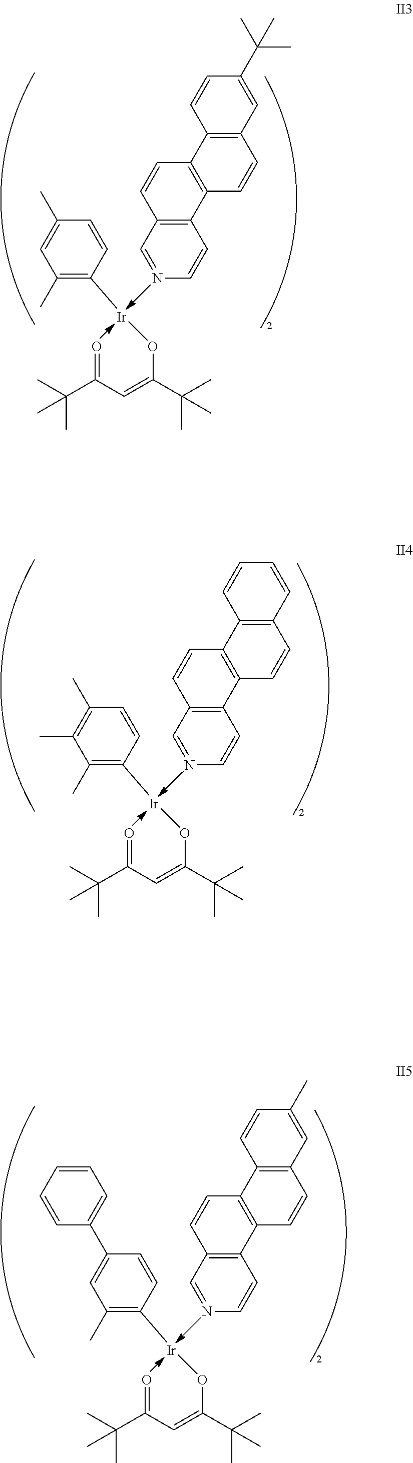

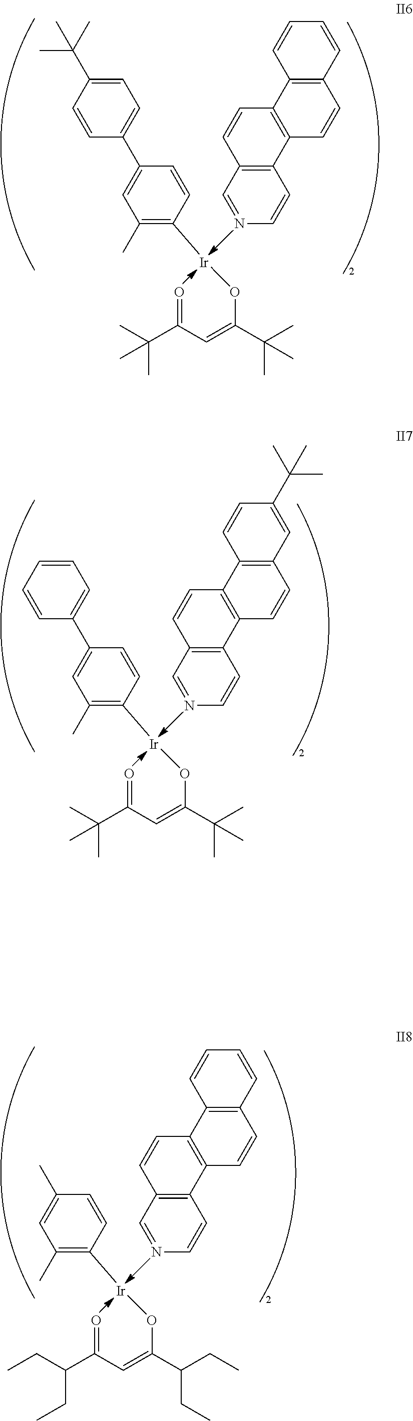

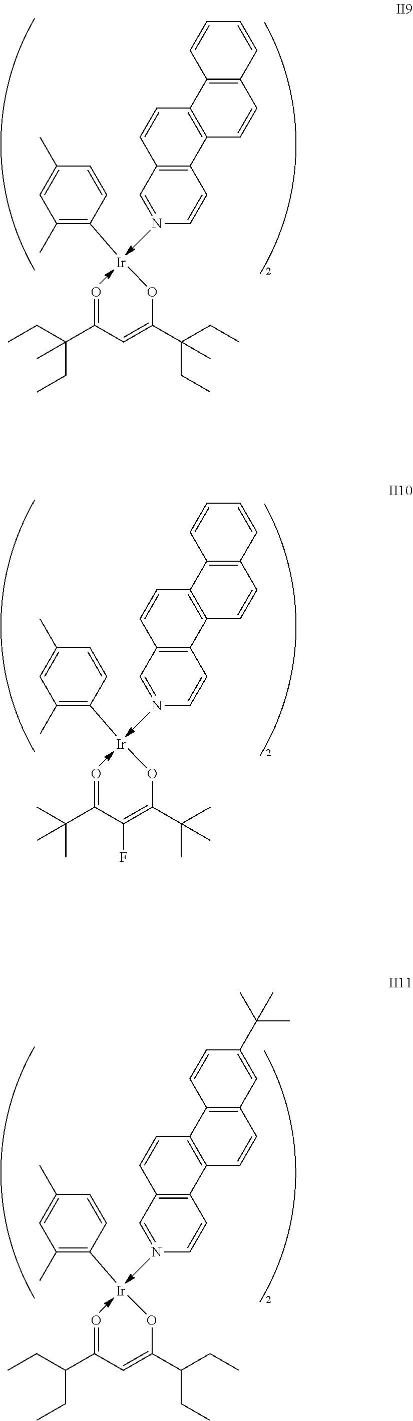

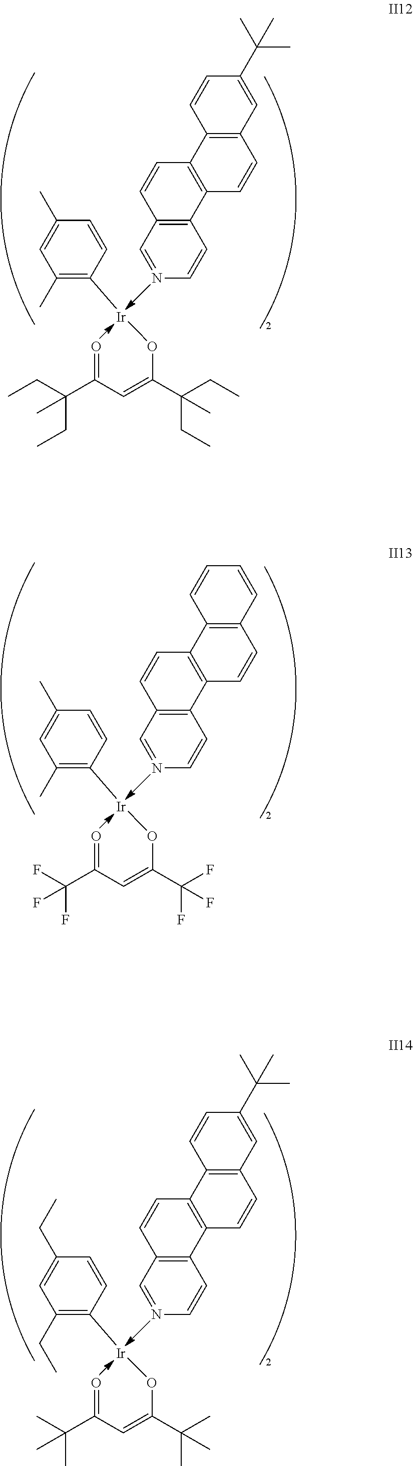

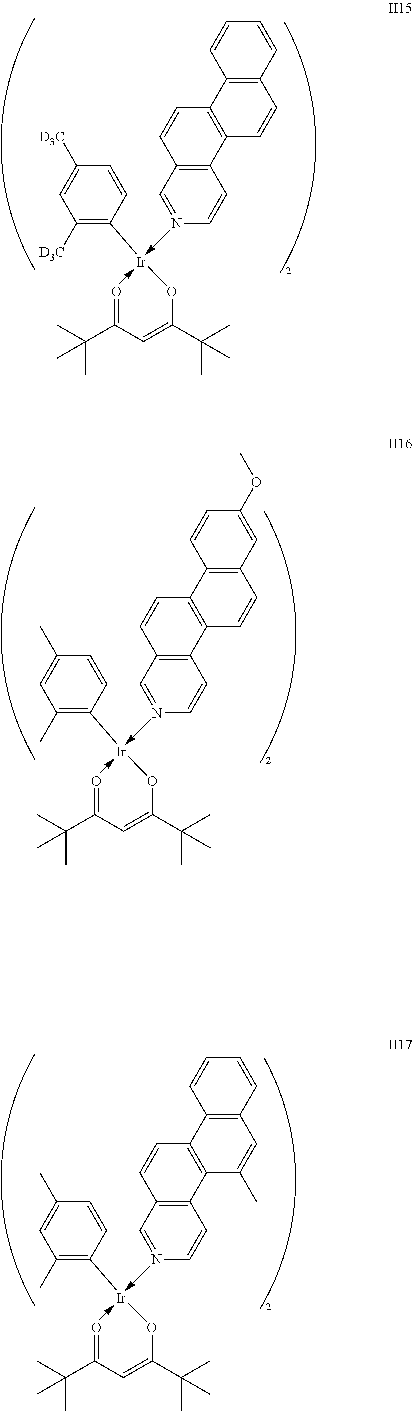

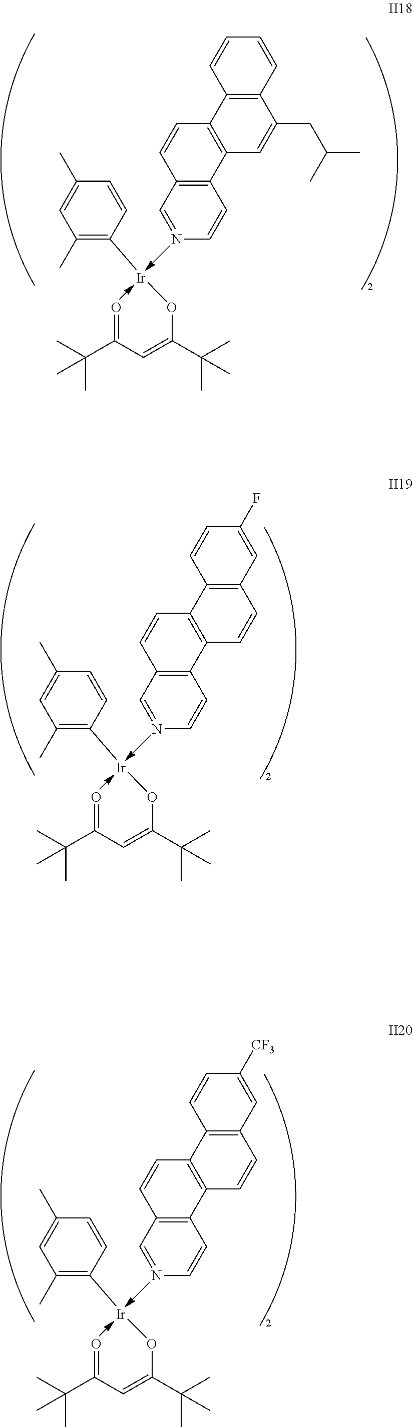

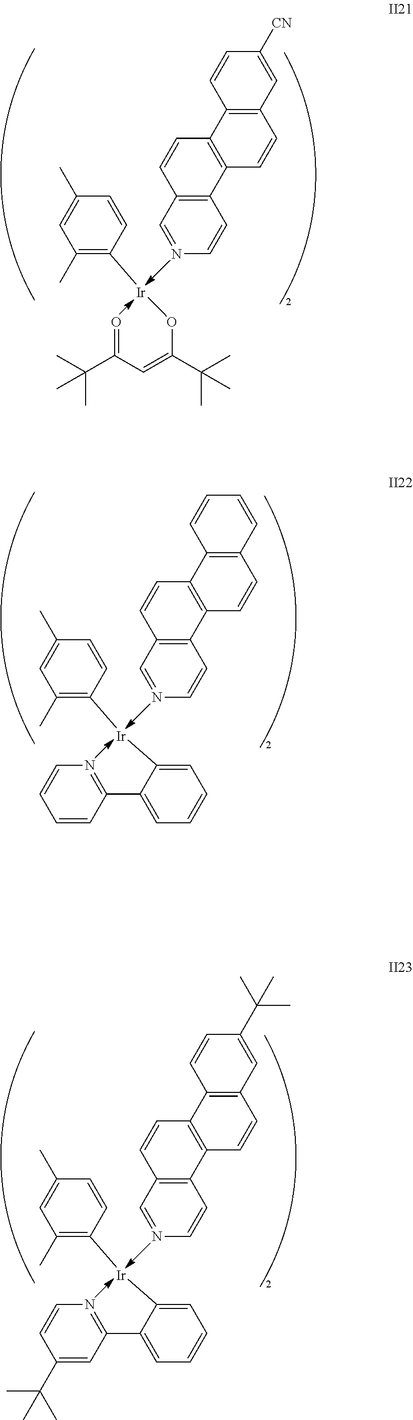

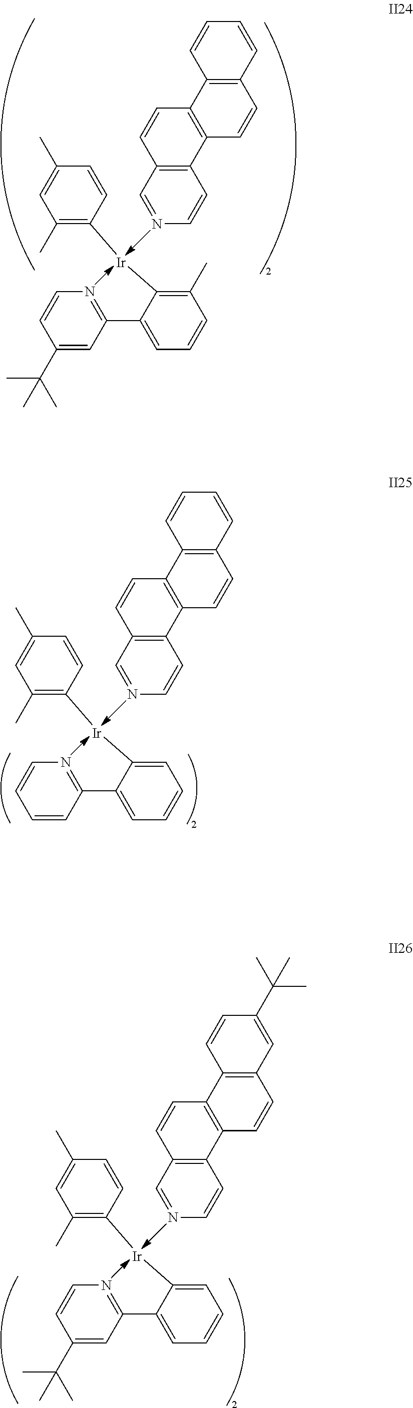

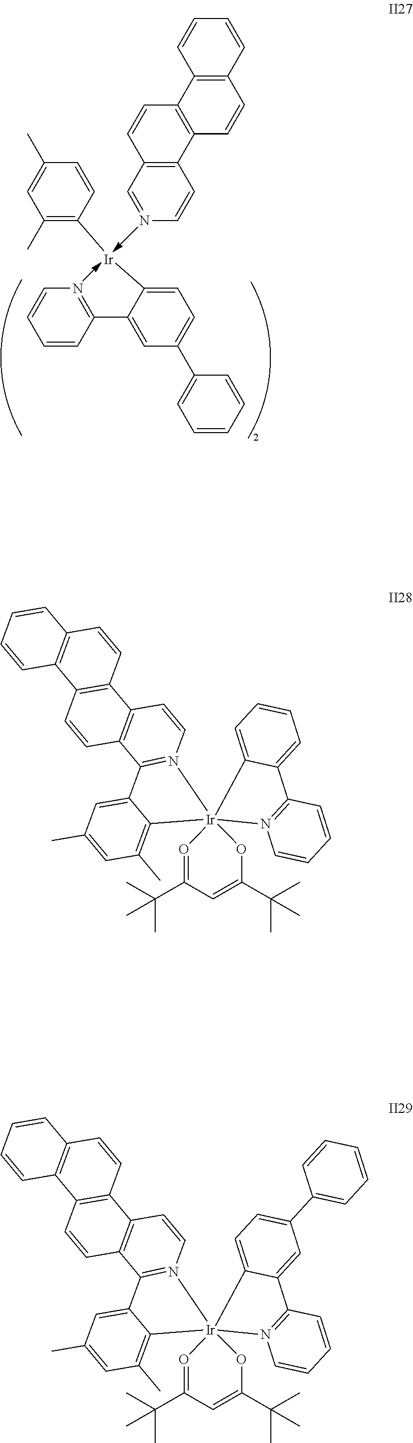

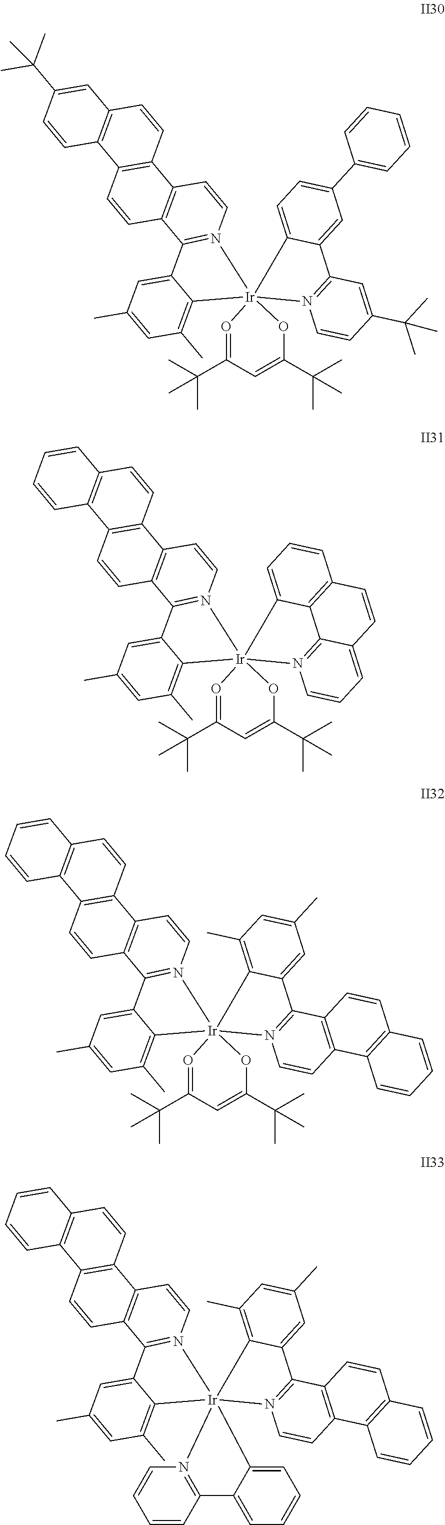

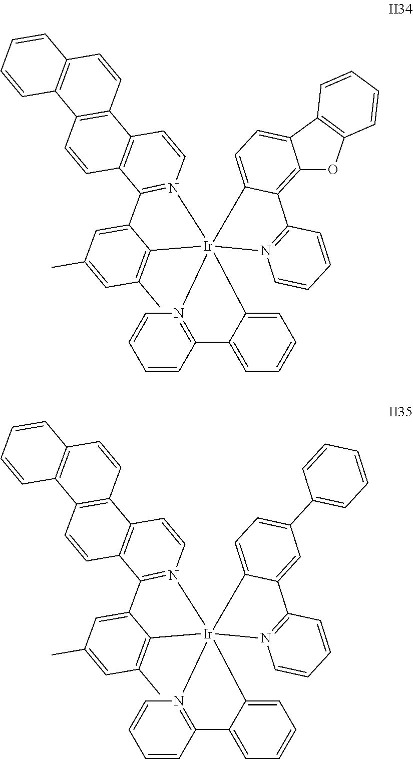

Among these organometallic complexes, exemplary compounds belonging to the groups AA and BB are compounds with at least a phenanthrene ring in a ligand of the Ir complex. Thus, the compounds have particularly high stability because the fused ring constituting the ligand has an SP2 hybrid orbital.

Among these organometallic complexes, the exemplary compounds belonging to the group CC are compounds with at least a triphenylene ring in a ligand of the Ir complex. Thus, the compounds have particularly high stability because the fused ring constituting the ligand has an SP2 hybrid orbital.

Among these organometallic complexes, the exemplary compounds belonging to the group DD are compounds with at least a dibenzofuran ring or a dibenzothiophene ring in a ligand of the Ir complex. Thus, these compounds contain an oxygen atom or a sulfur atom in a fused ring constituting the ligand. Abundant lone pairs in these atoms can enhance charge transport ability and make it particularly easy to adjust the carrier balance of the compounds.

Among these organometallic complexes, the exemplary compounds belonging to the groups EE and GG are compounds with at least a benzofluorene ring in a ligand of the Ir complex. Thus, these compounds have a substituent at position 9 of fluorene, have the substituent in the direction perpendicular to the in-plane direction of the fluorene ring, and can therefore particularly suppress the overlap between fused rings. Thus, the compounds have particularly high sublimability.

Among these organometallic complexes, the exemplary compounds belonging to the group HH are compounds with at least a benzoisoquinoline ring in a ligand of the Ir complex. Thus, these compounds are compounds in which the π-conjugation of the heterocyclic ring constituting the ligand is extended and promotes the interaction with a host.

Among these organometallic complexes, the exemplary compounds belonging to the group II are compounds with at least a naphthoisoquinoline ring in a ligand of the Ir complex. Thus, these compounds are compounds in which the 7r-conjugation of the heterocyclic ring constituting the ligand is extended and promotes the interaction with a host.

### <<Organic Light-Emitting Element>>

Next, an organic light-emitting element according to the present embodiment is described. The organic light-emitting element according to the present embodiment includes at least a pair of electrodes, a positive electrode and a negative electrode, and an organic compound layer between the electrodes. In the organic light-emitting element according to the present embodiment, the organic compound layer may be a single layer or a laminate of a plurality of layers, provided that the organic compound layer has a light-emitting layer. When the organic compound layer is a laminate of a plurality of layers, the organic compound layer may have a hole-injection layer, a hole-transport layer, an electron-blocking layer, a hole/exciton-blocking layer, an electron-transport layer, and/or an electron-injection layer, in addition to the light-emitting layer. The light-emitting layer may be a single layer or a laminate of a plurality of layers.

In the organic light-emitting element according to the present embodiment, at least one layer of the organic compound layers contains the organic compound according to the present embodiment. More specifically, the organic compound according to the present embodiment is contained in one of the light-emitting layer, the hole-injection layer, the hole-transport layer, the electron-blocking layer, the hole/exciton-blocking layer, the electron-transport layer, the electron-injection layer, and the like. The organic compound according to the present embodiment can be contained in the light-emitting layer.

In the organic light-emitting element according to the present embodiment, when the organic compound according to the present embodiment is contained in the light-emitting layer, the light-emitting layer may be composed only of the organic compound according to the present embodiment or may be composed of the organic compound according to the present embodiment and another compound. When the light-emitting layer is composed of the organic compound according to the present embodiment and another compound, the organic compound according to the present embodiment may be used as a host or a guest of the light-emitting layer. The compound may also be used as an assist material that may be contained in the light-emitting layer. The host is a compound with the highest mass ratio among the compounds constituting the light-emitting layer. The guest is a compound that has a lower mass ratio than the host among the compounds constituting the light-emitting layer and that is a principal light-emitting compound. The assist material is a compound that has a lower mass ratio than the host among the compounds constituting the light-emitting layer and that assists the guest in emitting light. The assist material is also referred to as a second host.

When the organic compound according to the present embodiment is used as a host in a light-emitting layer, the concentration of the host preferably ranges from 50% to 99% by mass, more preferably 70% to 99% by mass, of the entire light-emitting layer.

When the organic compound according to the present embodiment is used as an assist material in a light-emitting layer, the concentration of the assist material preferably ranges from 10% to 49% by mass, more preferably 15% to 35% by mass, of the entire light-emitting layer.

When the organic compound according to the present embodiment is used as a guest in a light-emitting layer, the concentration of the guest preferably ranges from 1% to 20% by mass, more preferably 5% to 15% by mass, of the entire light-emitting layer.

The present inventors have conducted various studies and have found that the organic compound according to the present embodiment can be used as a host or an assist material in a light-emitting layer, particularly as a host in the light-emitting layer, to provide an element that can efficiently emit bright light and that has very high durability. The light-emitting layer may be monolayer or multilayer. Green light emission may be chosen as an emission color of the present embodiment for color mixture with a light-emitting material of another emission color. The term “multilayer”, as used herein, refers to a state in which a first light-emitting layer and a light-emitting layer different from the first light-emitting layer are stacked. The first light-emitting layer and a second light-emitting layer may emit light of different colors. The emission color of the organic light-emitting element is not limited to green. More specifically, the emission color may be white or a neutral color. For white-light emission, the second light-emitting layer emits light of a color other than green, that is, blue or red. Such a layer is formed by vapor deposition or coating. This is described in detail below in exemplary embodiments.

The organic compound according to the present embodiment can be used as a constituent material of an organic compound layer other than the light-emitting layer constituting the organic light-emitting element according to the present embodiment. More specifically, the organic compound according to the present embodiment may be used as a constituent material of an electron-transport layer, an electron-injection layer, a hole-transport layer, a hole-injection layer, and/or a hole-blocking layer. In such a case, the emission color of the organic light-emitting element is not limited to green. More specifically, the emission color may be white or a neutral color.

If necessary, the organic compound according to the present embodiment may be used in combination with a known low-molecular-weight or high-molecular-weight hole-injection compound or hole-transport compound, host compound, light-emitting compound, electron-injection compound, or electron-transport compound. Examples of these compounds are described below.

The hole-injection/transport material can be a material with high hole mobility to facilitate the injection of holes from a positive electrode and to transport the injected holes to a light-emitting layer. Furthermore, a material with a high glass transition temperature can be used to reduce degradation of film quality, such as crystallization, in an organic light-emitting element. Examples of the low-molecular-weight or high-molecular-weight material with hole-injection/transport ability include, but are not limited to, triarylamine derivatives, aryl carbazole derivatives, phenylenediamine derivatives, stilbene derivatives, phthalocyanine derivatives, porphyrin derivatives, polyvinylcarbazole, polythiophene, and other electrically conductive polymers. The hole-injection/transport material can also be used for an electron-blocking layer. Specific examples of compounds that can be used as hole-injection/transport materials include, but are not limited to, the following.

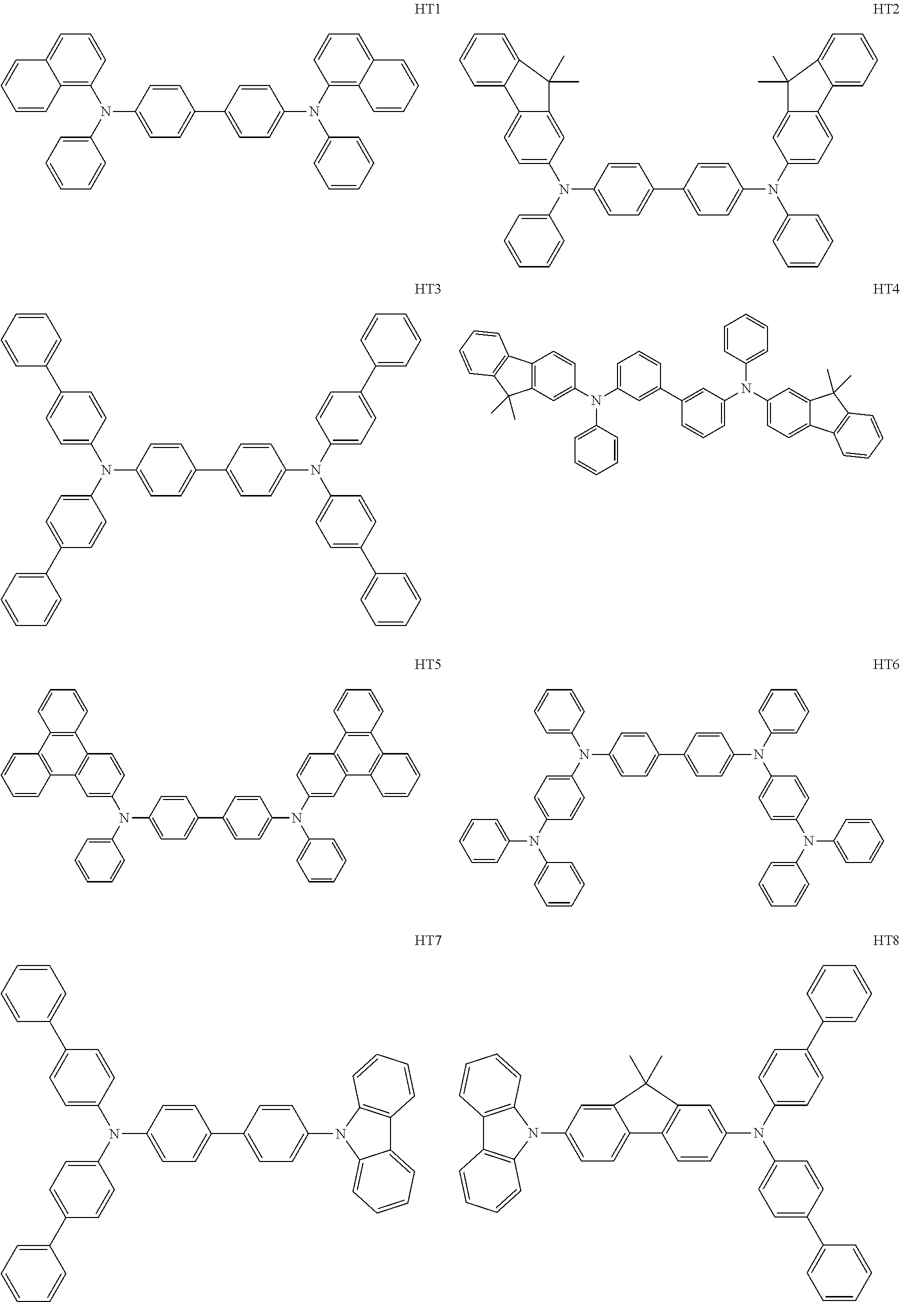

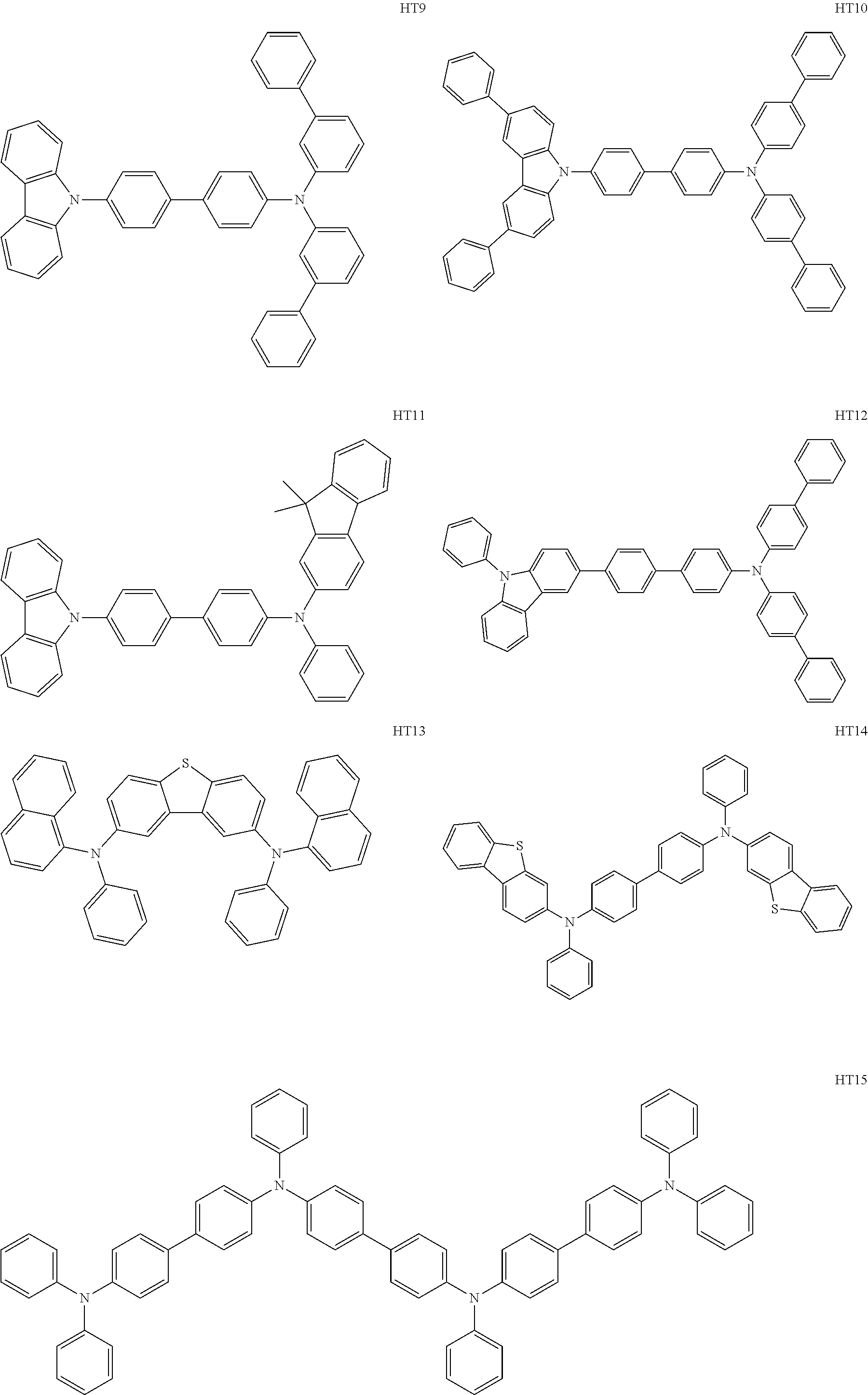

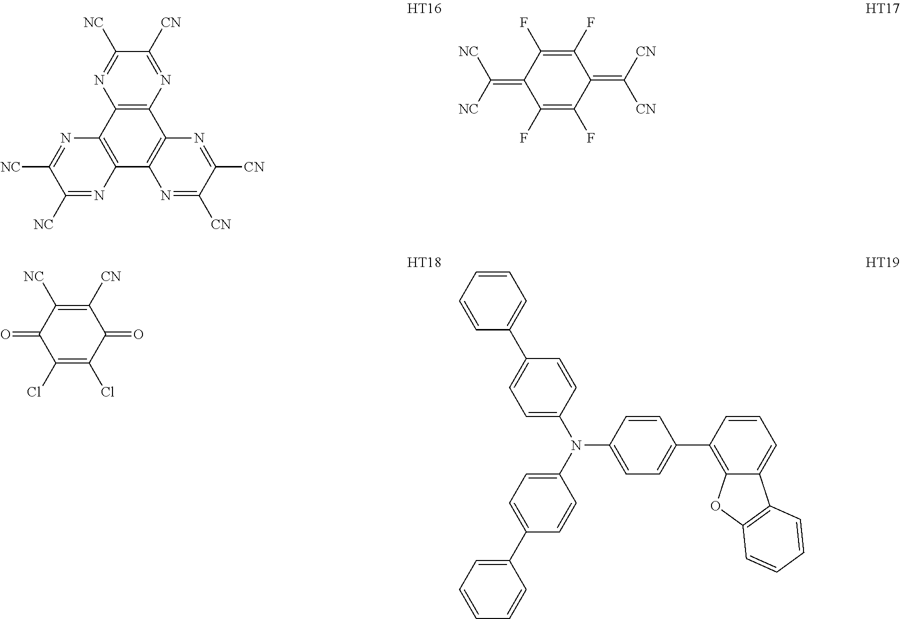 [SMILES: N#Cc1nc2c3nc(C#N)c(C#N)nc3c3nc(C#N)c(C#N)nc3c2nc1C#N ; N#CC(C#N)=c1c(F)c(F)c(=C(C#N)C#N)c(F)c1F ; N#CC1=C(C#N)C(=O)C(Cl)=C(Cl)C1=O ; c1ccc(-c2ccc(N(c3ccc(-c4ccccc4)cc3)c3ccc(-c4cccc5c4oc4ccccc45)cc3)cc2)cc1]

Examples of a light-emitting material mainly related to the light-emitting function include, in addition to the organic compounds represented by the general formulae [Ir-1] to [Ir-11], fused-ring compounds (for example, fluorene derivatives, naphthalene derivatives, pyrene derivatives, perylene derivatives, tetracene derivatives, anthracene derivatives, rubrene, etc.), quinacridone derivatives, coumarin derivatives, stilbene derivatives, organoaluminum complexes, such as tris(8-quinolinolato)aluminum, iridium complexes, platinum complexes, rhenium complexes, copper complexes, europium complexes, ruthenium complexes, and polymer derivatives, such as poly(phenylene vinylene) derivatives, polyfluorene derivatives, and polyphenylene derivatives.

Specific examples of compounds that can be used as light-emitting materials include, but are not limited to, the following.

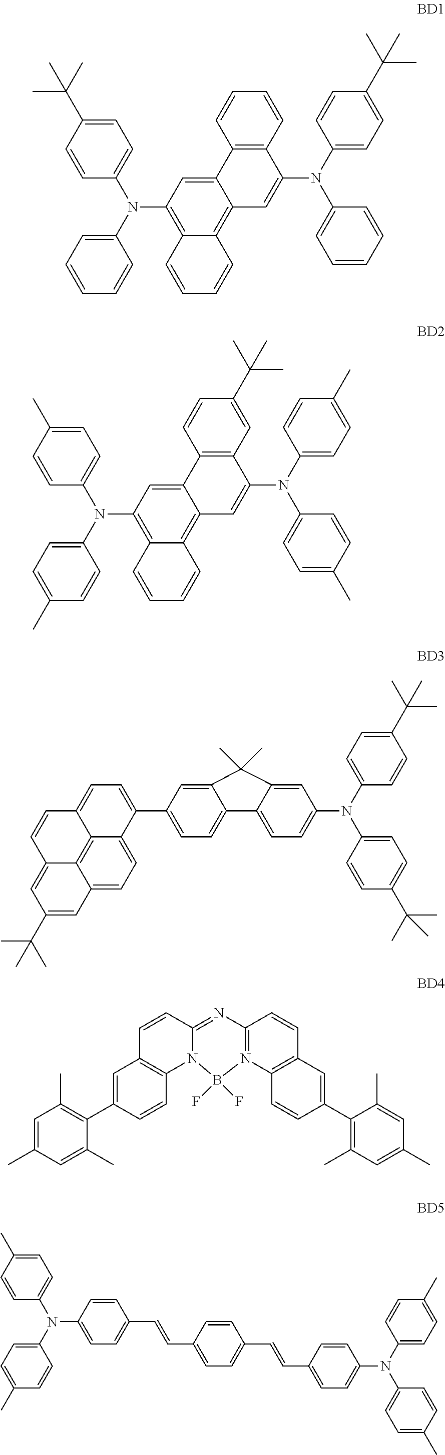

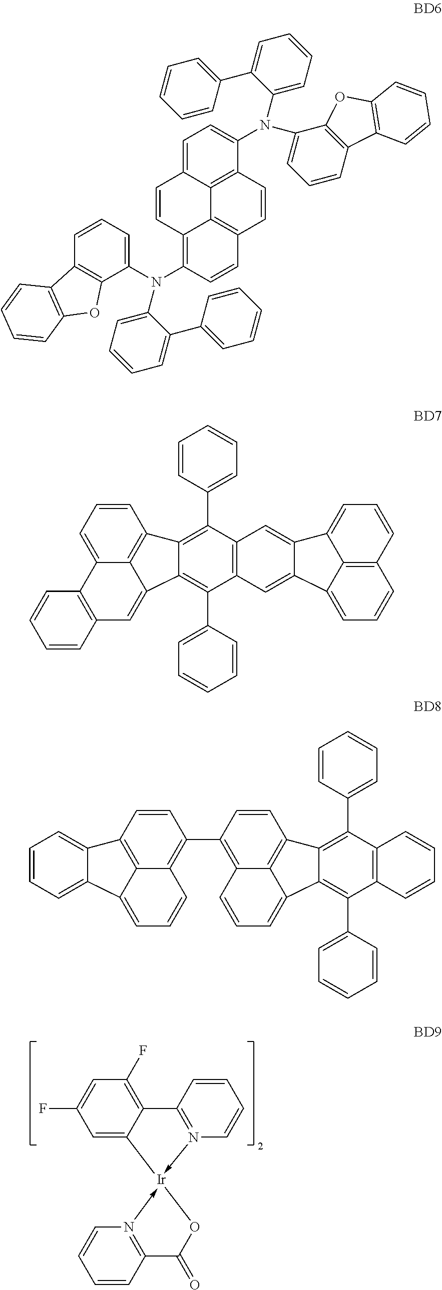

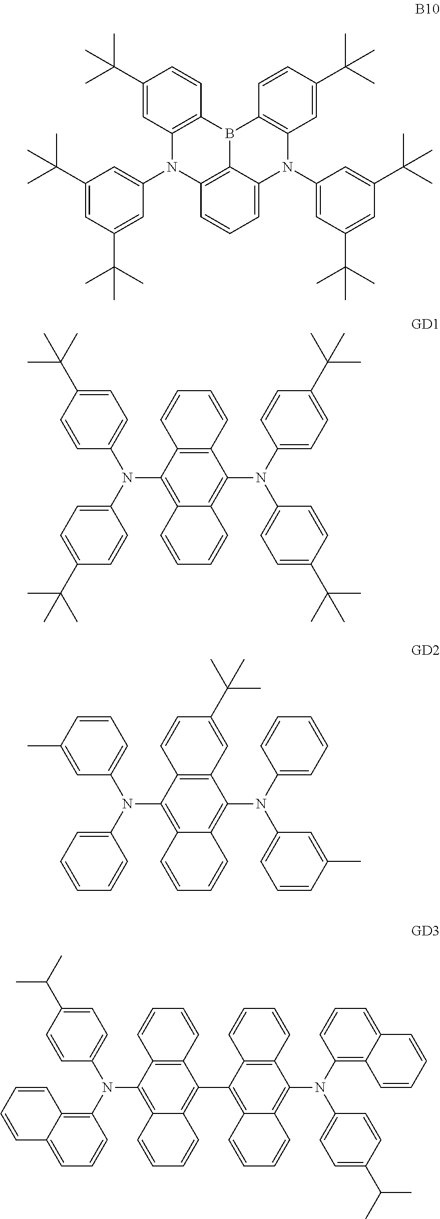

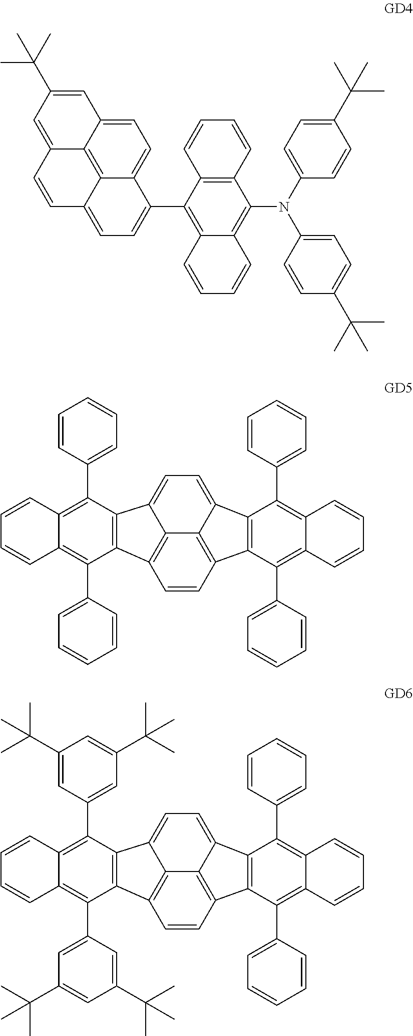

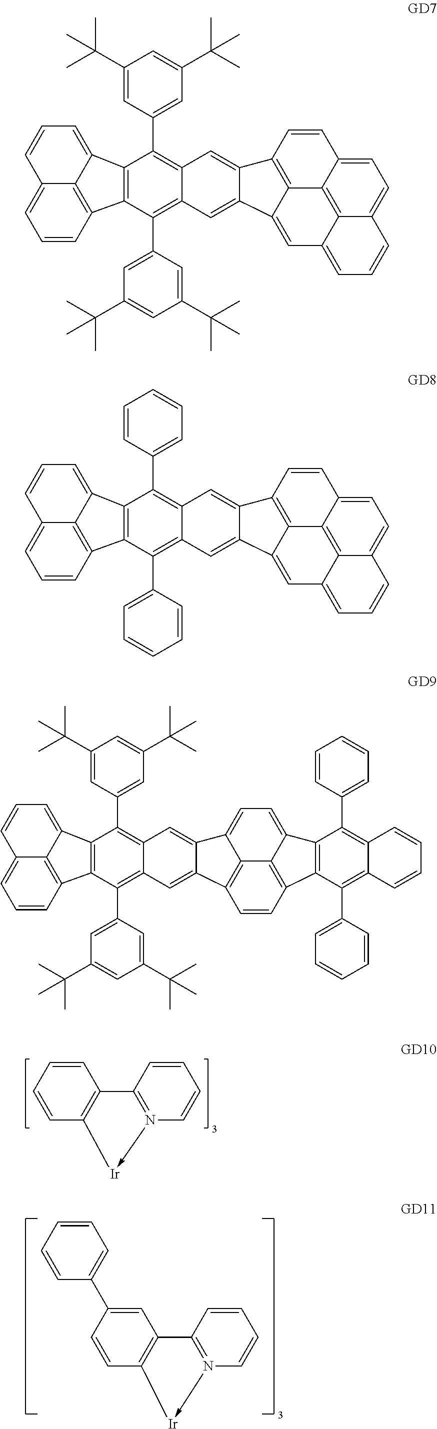

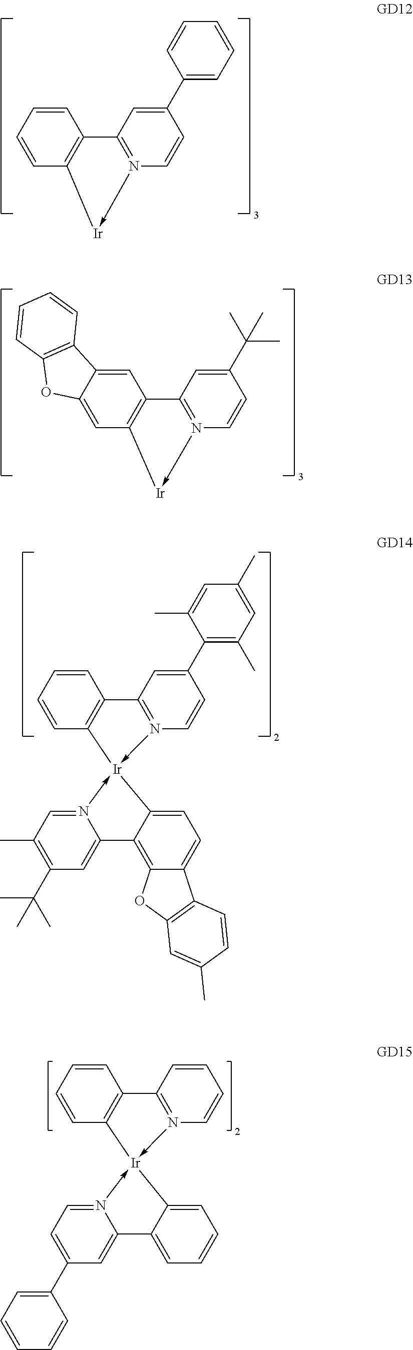

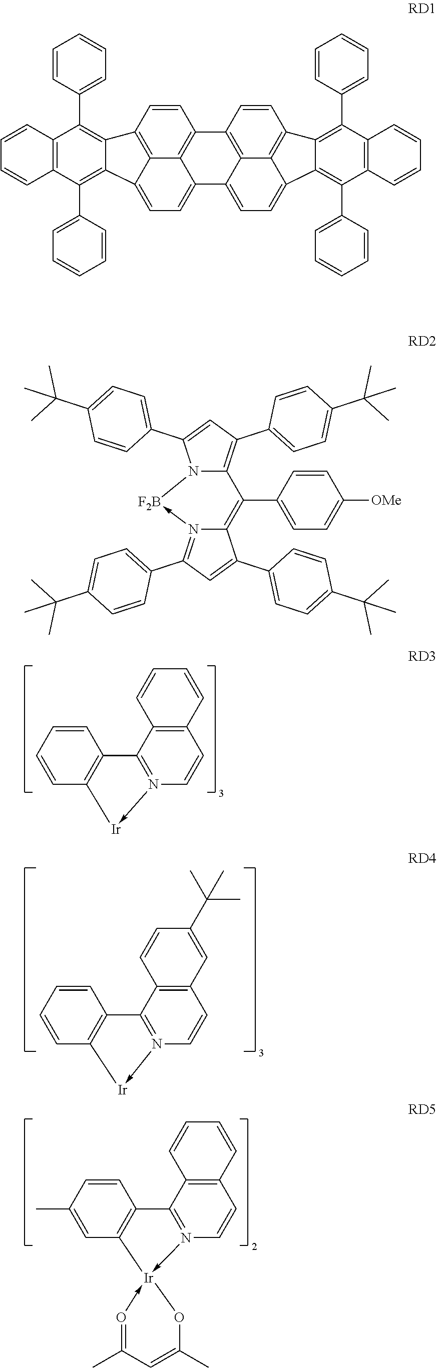

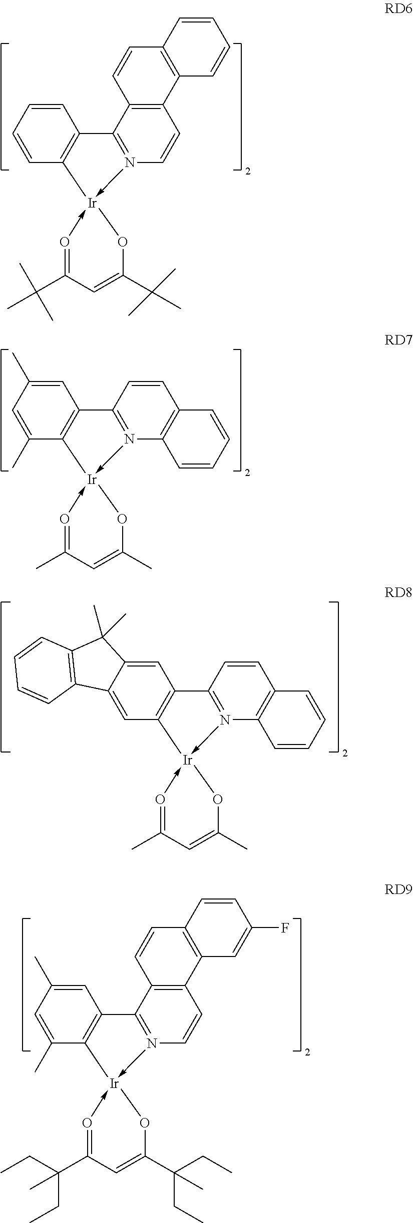

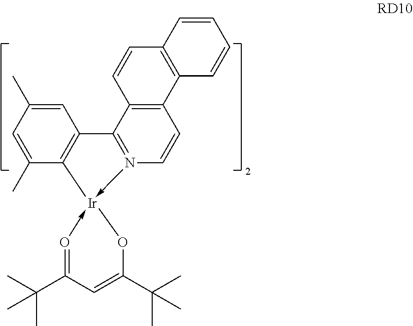

Examples of a light-emitting layer host or a light-emitting assist material in a light-emitting layer include aromatic hydrocarbon compounds and derivatives thereof, carbazole derivatives, dibenzofuran derivatives, dibenzothiophene derivatives, organoaluminum complexes, such as tris(8-quinolinolato)aluminum, and organoberyllium complexes, in addition to the organic compound according to the present embodiment represented by the general formula [1].

In particular, a material with a carbazole skeleton, a material with an azine ring in the skeleton, or a material with xanthone in the skeleton as an assist material can be used as a third component in a light-emitting layer containing the organic compound according to the present embodiment and the phosphorescent material. This is because these materials have high electron-donating ability and electron-withdrawing ability, and the HOMO and LUMO levels can be easily adjusted.

The organic compound according to the present embodiment is a compound in which an electron-donating moiety composed of a cross-linked triphenylamine is bonded to an electron-withdrawing moiety composed of a xanthone skeleton. Thus, although the organic compound is useful as a bipolar host, an assist material can be added depending on the carrier balance of the entire element. A material with a skeleton that can adjust the HOMO or LUMO level can be used as an assist material. Such an assist material in combination with the organic compound according to the present embodiment can achieve a good carrier balance.

Specific examples of a compound that can be used as a light-emitting layer host or a light-emitting assist material in a light-emitting layer include, but are not limited to, the following.

Among the following specific examples, materials with a carbazole skeleton that can be used as assist materials are EM32 to EM38. Materials with an azine ring in the skeleton that can be used as assist materials are EM35 to EM40. Materials with xanthone in the skeleton that can be used as assist materials are EM28 and EM30.

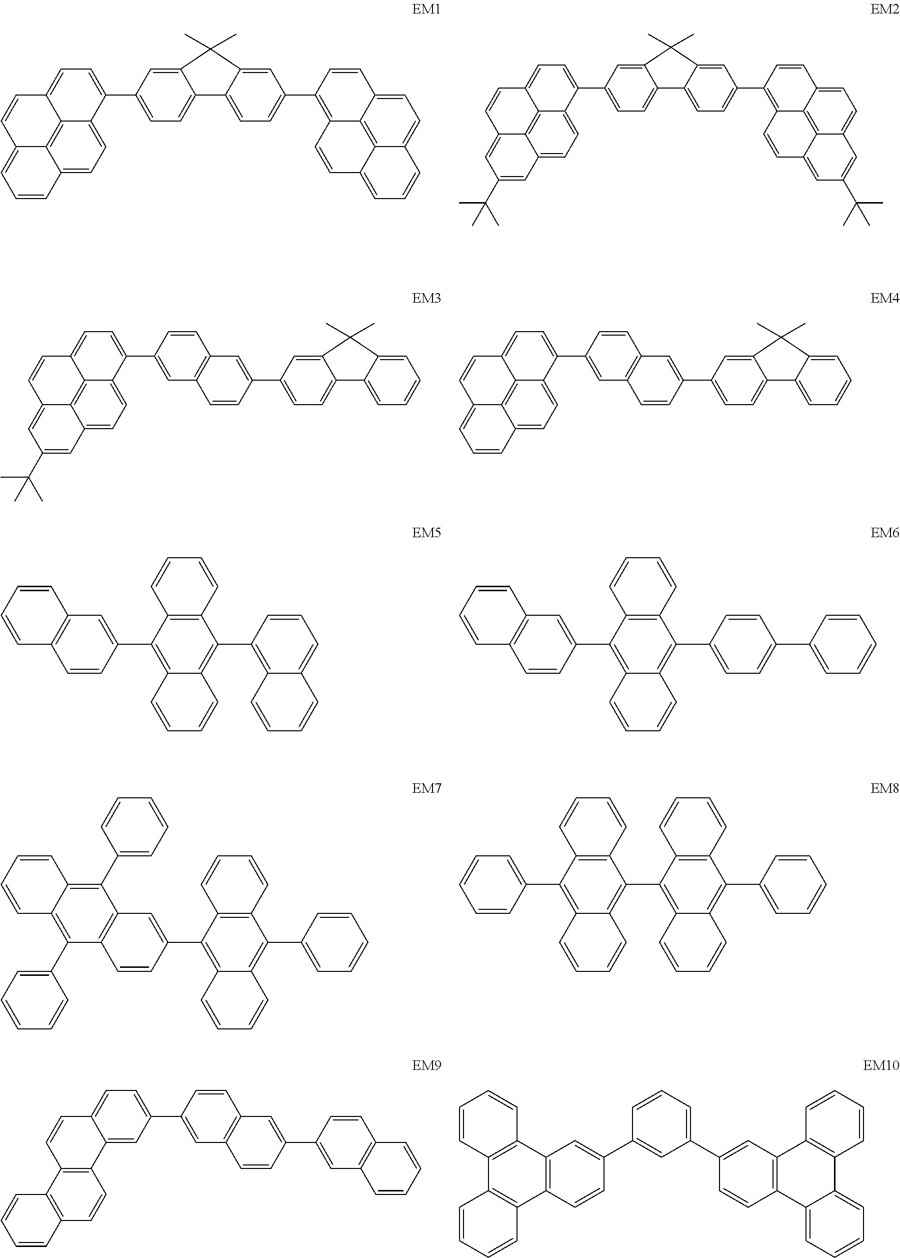

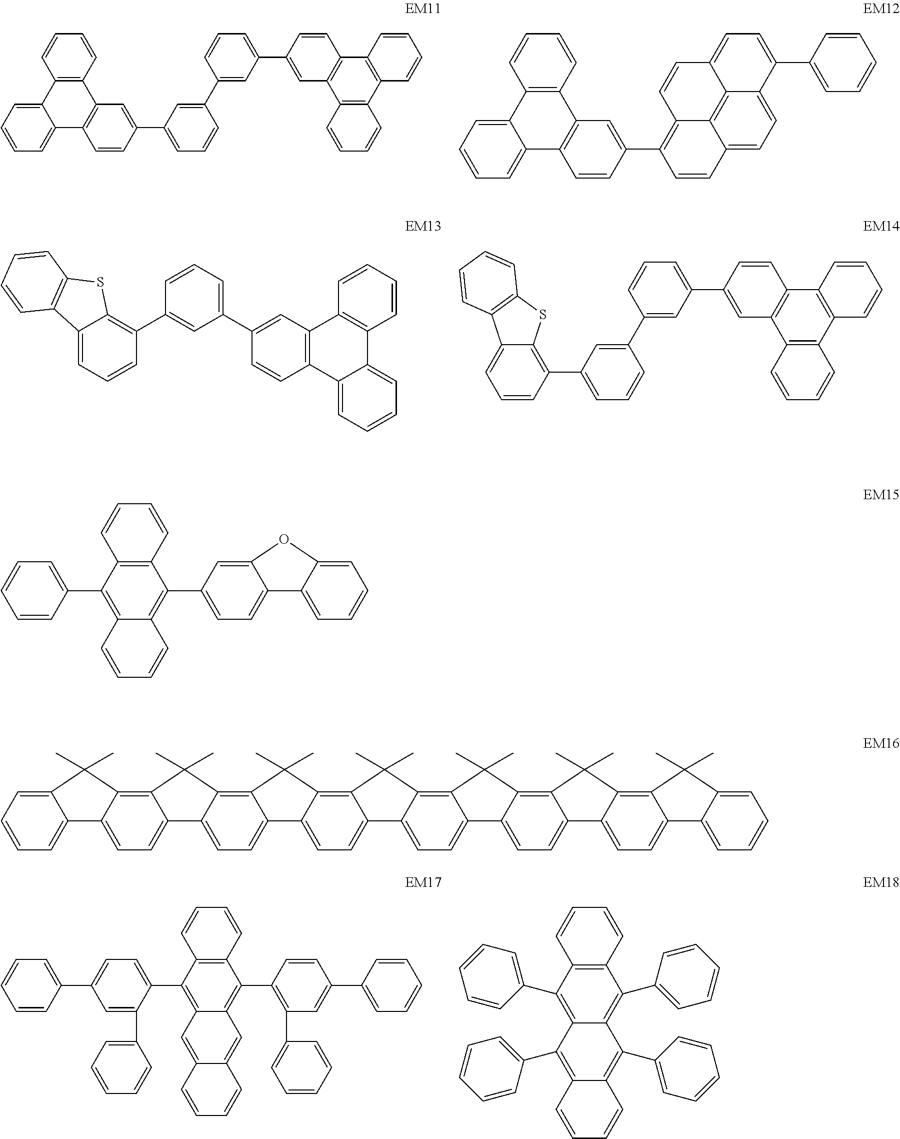

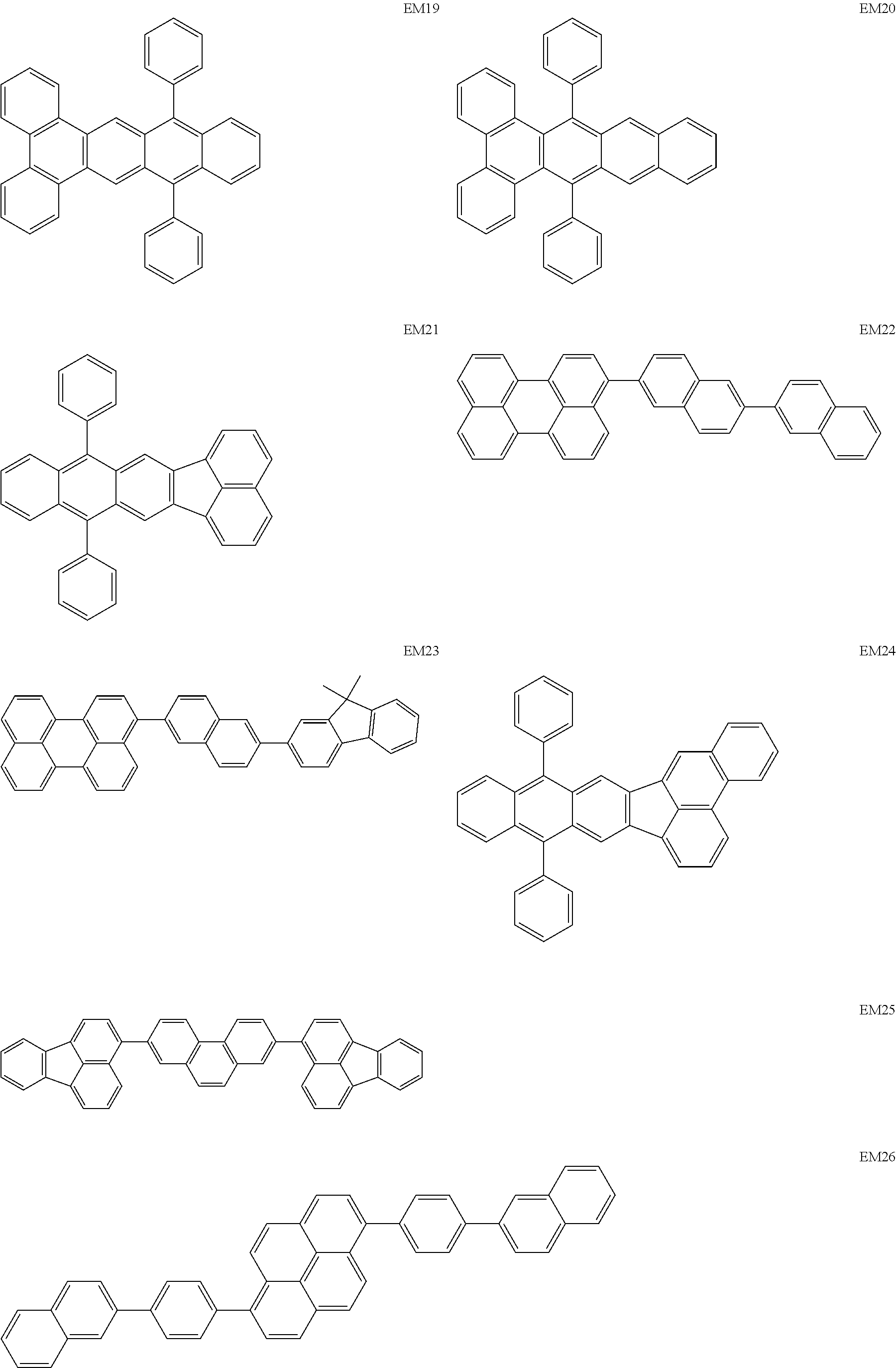

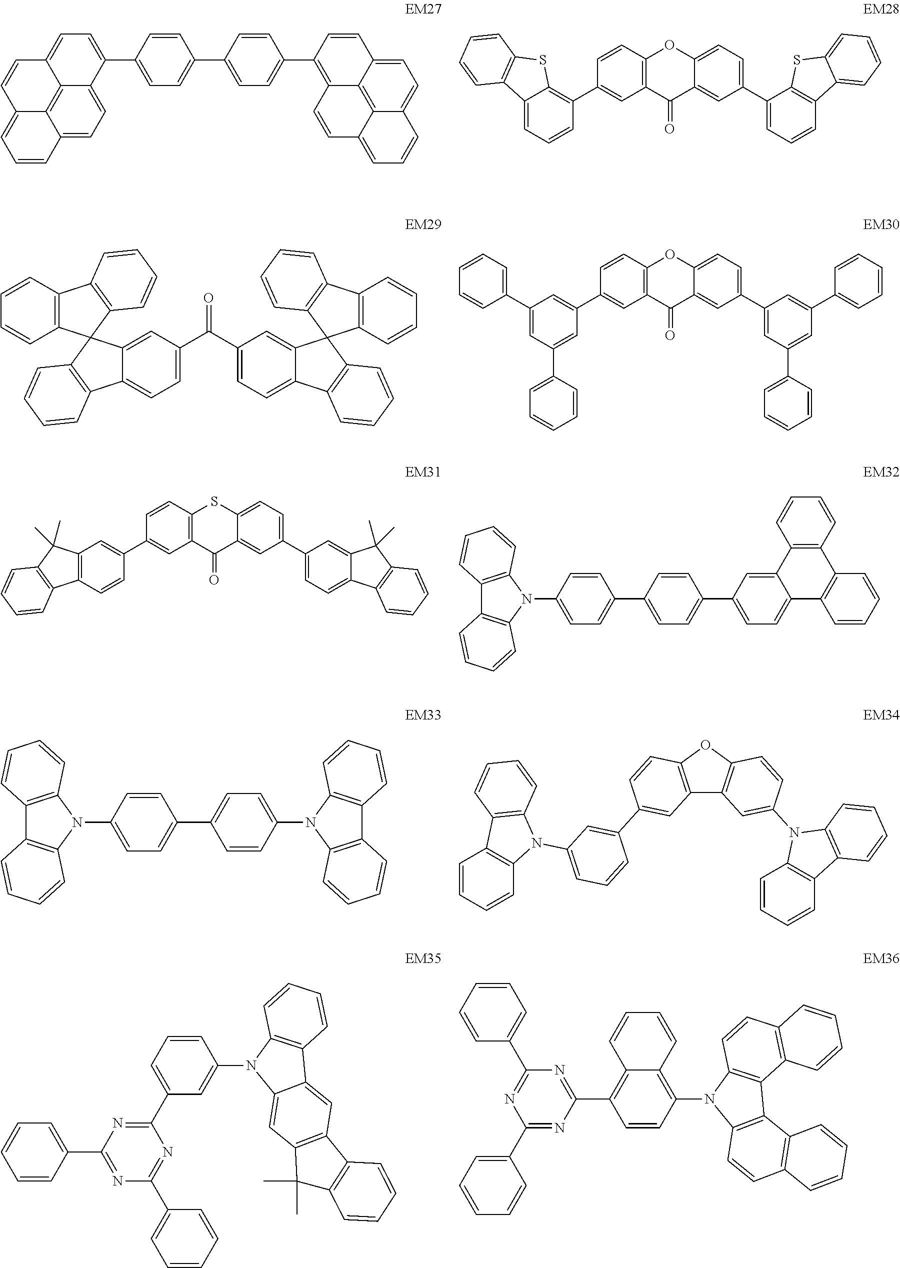

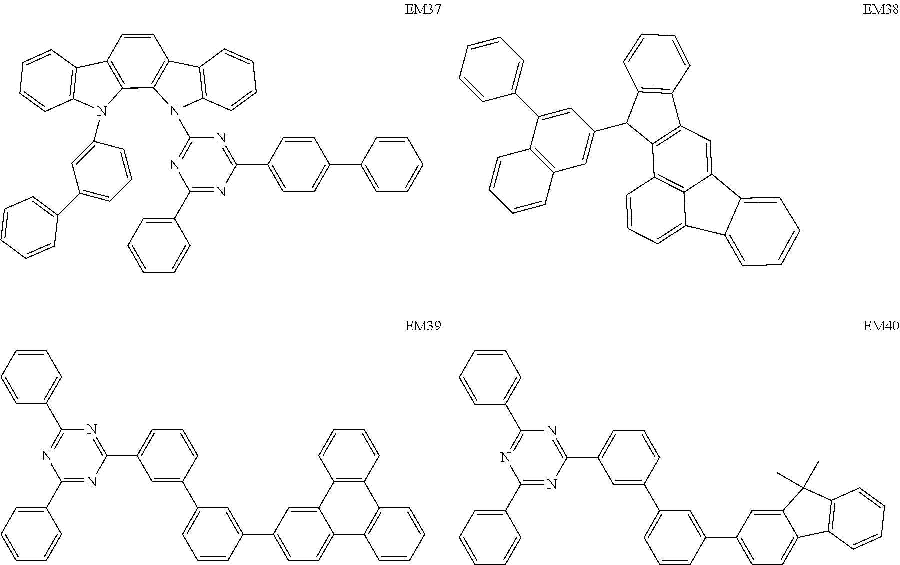

An electron-transport material can be at least one selected from the group consisting of materials that can transport electrons injected from the negative electrode to the light-emitting layer and is selected in consideration of the balance with the hole mobility of a hole-transport material and the like. Examples of materials with electron-transport ability include, but are not limited to, oxadiazole derivatives, oxazole derivatives, pyrazine derivatives, triazole derivatives, triazine derivatives, quinoline derivatives, quinoxaline derivatives, phenanthroline derivatives, organoaluminum complexes, and fused-ring compounds (for example, fluorene derivatives, naphthalene derivatives, chrysene derivatives, and anthracene derivatives). Furthermore, the electron-transport material is also suitably used for a hole-blocking layer.

Specific examples of compounds that can be used as electron-transport materials include, but are not limited to, the following.

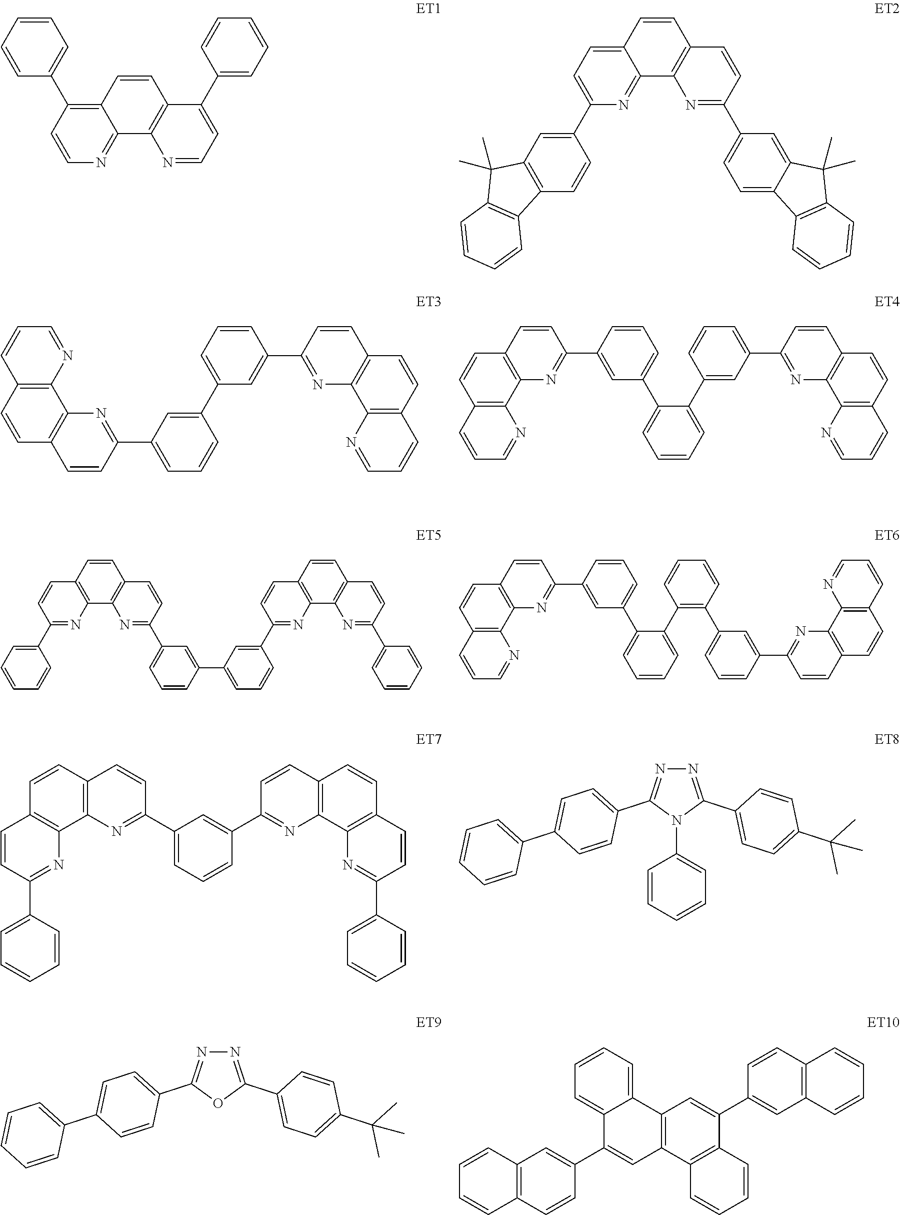

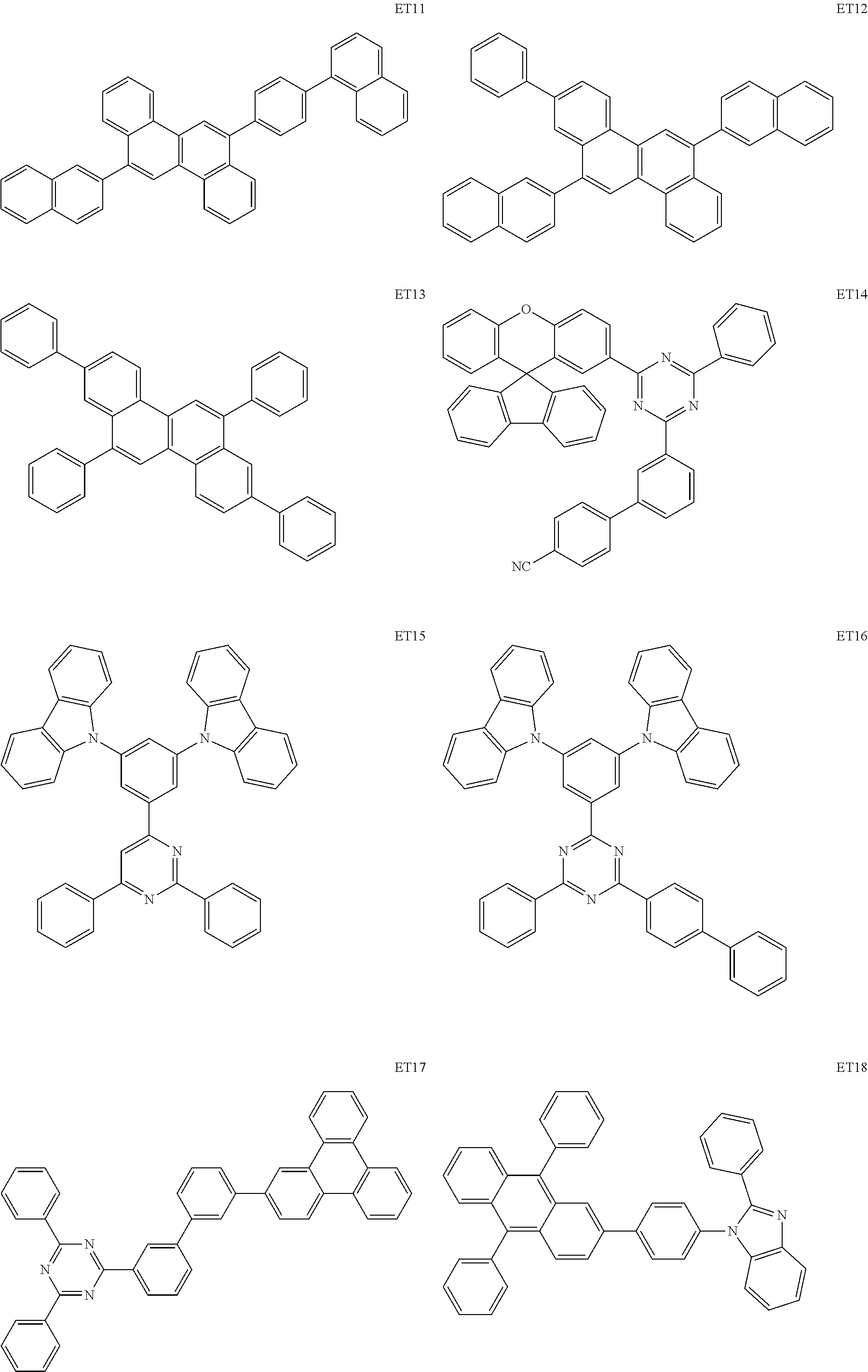

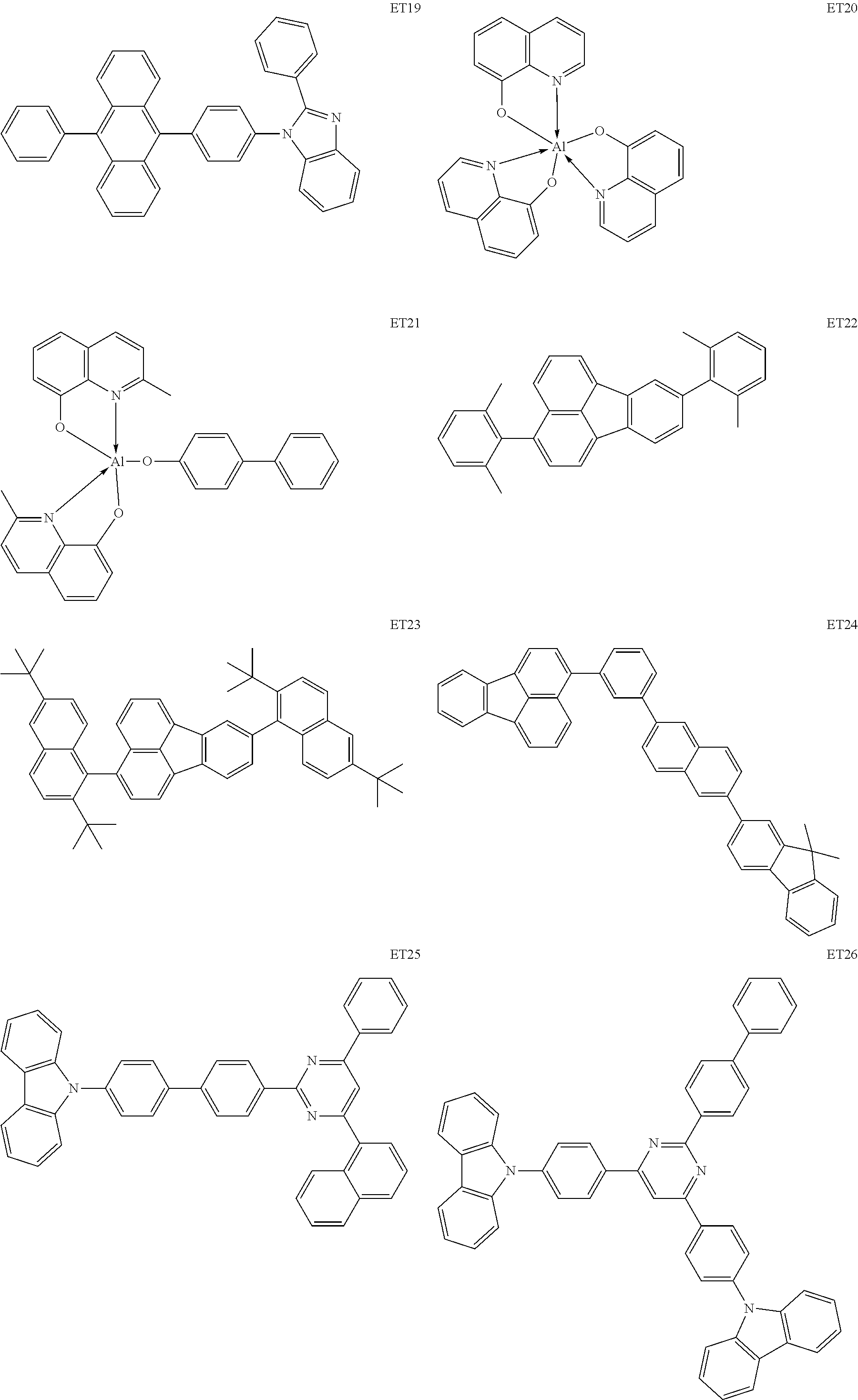

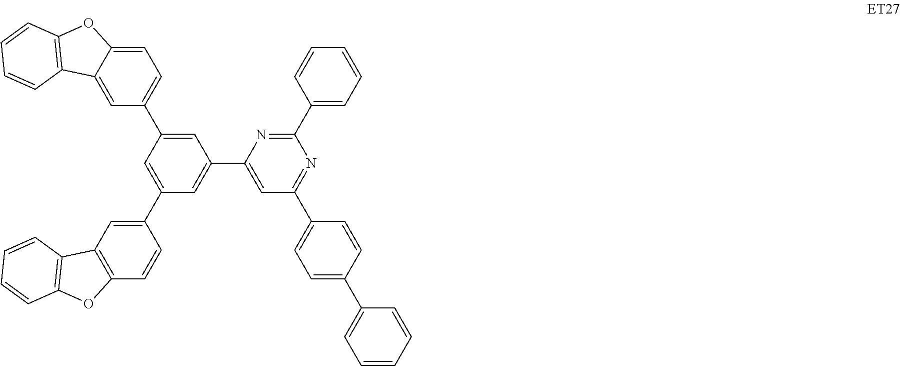 [SMILES: c1ccc(-c2ccc(-c3cc(-c4cc(-c5ccc6oc7ccccc7c6c5)cc(-c5ccc6oc7ccccc7c6c5)c4)nc(-c4ccccc4)n3)cc2)cc1]

### [Structure of Organic Light-Emitting Element]

An organic light-emitting element includes an insulating layer, a first electrode, an organic compound layer, and a second electrode on a substrate. A protective layer, a color filter, a microlens, or the like may be provided on the negative electrode.

When a color filter is provided, a planarization layer may be provided between the color filter and a protective layer. The planarization layer may be composed of an acrylic resin or the like. The same applies to a planarization layer provided between a color filter and a microlens.

### [Substrate]

The substrate may be formed of quartz, glass, silicon wafer, resin, metal, or the like. The substrate may have a switching element, such as a transistor, and wiring, on which an insulating layer may be provided. The insulating layer may be composed of any material, provided that the insulating layer can have a contact hole for wiring between the insulating layer and the first electrode and is insulated from unconnected wires. For example, the insulating layer may be formed of a resin, such as polyimide, silicon oxide, or silicon nitride.

### [Electrode]

A pair of electrodes can be used as electrodes. The pair of electrodes may be a positive electrode and a negative electrode.

When an electric field is applied in a direction in which the organic light-emitting element emits light, an electrode with a high electric potential is a positive electrode, and the other electrode is a negative electrode. In other words, the electrode that supplies holes to the light-emitting layer is a positive electrode, and the electrode that supplies electrons is a negative electrode.

A constituent material of the positive electrode can have as large a work function as possible. Examples of the constituent material include metal elements, such as gold, platinum, silver, copper, nickel, palladium, cobalt, selenium, vanadium, and tungsten, mixtures thereof, alloys thereof, and metal oxides, such as tin oxide, zinc oxide, indium oxide, indium tin oxide (ITO), and indium zinc oxide. Electrically conductive polymers, such as polyaniline, polypyrrole, and polythiophene, may also be used.

These electrode materials may be used alone or in combination. The positive electrode may be composed of a single layer or a plurality of layers.

When used as a reflective electrode, for example, chromium, aluminum, silver, titanium, tungsten, molybdenum, an alloy thereof, or a laminate thereof can be used. These materials can also function as a reflective film that does not have a role as an electrode. When used as a transparent electrode, an oxide transparent conductive layer, such as indium tin oxide (ITO) or indium zinc oxide, can be used. However, the present disclosure is not limited thereto. The electrodes may be formed by photolithography.

A constituent material of the negative electrode can be a material with a small work function. For example, an alkali metal, such as lithium, an alkaline-earth metal, such as calcium, a metal element, such as aluminum, titanium, manganese, silver, lead, or chromium, or a mixture thereof may be used. An alloy of these metal elements may also be used. For example, magnesium-silver, aluminum-lithium, aluminum-magnesium, silver-copper, or zinc-silver may be used. A metal oxide, such as indium tin oxide (ITO), may also be used. These electrode materials may be used alone or in combination. The negative electrode may be composed of a single layer or a plurality of layers. Among them, silver can be used, and a silver alloy can be used to reduce the aggregation of silver. As long as the aggregation of silver can be reduced, the alloy may have any ratio. For example, the ratio of silver to another metal may be 1:1, 3:1, or the like.

The negative electrode may be, but is not limited to, an oxide conductive layer, such as ITO, for a top emission element or a reflective electrode, such as aluminum (A1), for a bottom emission element. The negative electrode may be formed by any method. A direct-current or alternating-current sputtering method can achieve good film coverage and easily decrease resistance.

### [Organic Compound Layer]

The organic compound layer may be formed of a single layer or a plurality of layers. Depending on their functions, a plurality of layers may be referred to as a hole-injection layer, a hole-transport layer, an electron-blocking layer, a light-emitting layer, a hole-blocking layer, an electron-transport layer, or an electron-injection layer. The organic compound layer is composed mainly of an organic compound and may contain an inorganic atom or an inorganic compound. For example, the compound layer may contain copper, lithium, magnesium, aluminum, iridium, platinum, molybdenum, zinc, or the like. The organic compound layer may be located between the first electrode and the second electrode and may be in contact with the first electrode and the second electrode.

### [Protective Layer]

A protective layer may be provided on the negative electrode. For example, a glass sheet with a moisture absorbent may be attached to the negative electrode to decrease the amount of water or the like entering the organic compound layer and to reduce the occurrence of display defects. In another embodiment, a passivation film of silicon nitride or the like may be provided on the negative electrode to decrease the amount of water or the like entering the organic compound layer. For example, after the negative electrode is formed, the negative electrode is transferred to another chamber without breaking the vacuum, and a silicon nitride film with a thickness of 2 μm may be formed as a protective layer by a chemical vapor deposition (CVD) method. The protective layer may be formed by the CVD method followed by an atomic layer deposition (ALD) method. A film formed by the ALD method may be formed of any material such as silicon nitride, silicon oxide, or aluminum oxide. Silicon nitride may be further deposited by the CVD method on the film formed by the ALD method. The film formed by the ALD method may have a smaller thickness than the film formed by the CVD method. More specifically, the thickness may be 50% or less or even 10% or less.

### [Color Filter]

A color filter may be provided on the protective layer. For example, a color filter that matches the size of the organic light-emitting element may be provided on another substrate and may be bonded to the substrate on which the organic light-emitting element is provided, or a color filter may be patterned on the protective layer by photolithography. The color filter may be composed of a polymer.

### [Planarization Layer]

A planarization layer may be provided between the color filter and the protective layer. The planarization layer is provided to reduce the roughness of the underlayer. The planarization layer is sometimes referred to as a material resin layer with any purpose. The planarization layer may be composed of an organic compound and can be composed of a high-molecular-weight compound, though it may be composed of a low-molecular-weight compound. The planarization layer may be provided above and below the color filter, and the constituent materials thereof may be the same or different. Specific examples include polyvinylcarbazole resins, polycarbonate resins, polyester resins, ABS resins, acrylic resins, polyimide resins, phenolic resins, epoxy resins, silicone resins, and urea resins.

### [Microlens]

An organic light-emitting element may include an optical member, such as a microlens, on the light output side. The microlens may be composed of an acrylic resin, an epoxy resin, or the like. The microlens may be used to increase the amount of light extracted from the organic light-emitting element and to control the direction of the extracted light. The microlens may have a hemispherical shape. For a hemispherical microlens, the vertex of the microlens is a contact point between the hemisphere and a tangent line parallel to the insulating layer among the tangent lines in contact with the hemisphere. The vertex of the microlens in a cross-sectional view can be determined in the same manner. More specifically, the vertex of the microlens in a cross-sectional view is a contact point between the semicircle of the microlens and a tangent line parallel to the insulating layer among the tangent lines in contact with the semicircle.

The midpoint of the microlens can also be defined. In a cross section of the microlens, a midpoint of a line segment from one end point to the other end point of the arc can be referred to as a midpoint of the microlens. A cross section in which the vertex and the midpoint are determined may be perpendicular to the insulating layer.

### [Opposite Substrate]

An opposite substrate may be provided on the planarization layer. The opposite substrate is so called because it faces the substrate. The opposite substrate may be composed of the same material as the substrate. When the substrate is a first substrate, the opposite substrate may be a second substrate.

### [Organic Compound Layer]

An organic compound layer (a hole-injection layer, a hole-transport layer, an electron-blocking layer, a light-emitting layer, a hole-blocking layer, an electron-transport layer, an electron-injection layer, etc.) constituting the organic light-emitting element according to the present embodiment is formed by the following method.

An organic compound layer may be formed by a dry process, such as a vacuum deposit method, an ionized deposition method, sputtering, or plasma. Instead of the dry process, a wet process may also be employed in which a layer is formed by a known coating method (for example, spin coating, dipping, a casting method, an LB method, an ink jet method, etc.) using an appropriate solvent. A layer formed by a vacuum deposit method, a solution coating method, or the like undergoes little crystallization or the like and has high temporal stability. When a film is formed by a coating method, the film may also be formed in combination with an appropriate binder resin.

Examples of the binder resin include, but are not limited to, polyvinylcarbazole resins, polycarbonate resins, polyester resins, ABS resins, acrylic resins, polyimide resins, phenolic resins, epoxy resins, silicone resins, and urea resins.

These binder resins may be used alone or in combination as a homopolymer or a copolymer. If necessary, an additive agent, such as a known plasticizer, oxidation inhibitor, and/or ultraviolet absorbent, may also be used.

### [Pixel Circuit]

A light-emitting apparatus may include a pixel circuit coupled to the light-emitting element. The pixel circuit may be of an active matrix type, which independently controls the light emission of a first light-emitting element and a second light-emitting element. The active-matrix circuit may be voltage programmed or current programmed. The drive circuit has a pixel circuit for each pixel. The pixel circuit may include a light-emitting element, a transistor for controlling the luminance of the light-emitting element, a transistor for controlling light emission timing, a capacitor for holding the gate voltage of the transistor for controlling the luminance, and a transistor for GND connection without through the light-emitting element.

A light-emitting apparatus includes a display region and a peripheral region around the display region. The display region includes the pixel circuit, and the peripheral region includes a display control circuit. The mobility of a transistor constituting the pixel circuit may be smaller than the mobility of a transistor constituting the display control circuit.

The gradient of the current-voltage characteristics of a transistor constituting the pixel circuit may be smaller than the gradient of the current-voltage characteristics of a transistor constituting the display control circuit. The gradient of the current-voltage characteristics can be determined by so-called Vg-Ig characteristics. A transistor constituting the pixel circuit is a transistor coupled to a light-emitting element, such as a first light-emitting element.

### [Pixels]

An organic light-emitting apparatus including an organic light-emitting element may have a plurality of pixels. Each pixel has subpixels that emit light of different colors. For example, the subpixels may have RGB emission colors.

In each pixel, a region also referred to as a pixel aperture emits light. This region is the same as the first region. The pixel aperture may be 15 μm or less or 5 μm or more. More specifically, the pixel aperture may be 11 μm, 9.5 μm, 7.4 μm, or 6.4 μm. The distance between the subpixels may be 10 μm or less, more specifically, 8 μm, 7.4 μm, or 6.4 μm.

The pixels may be arranged in a known form in a plan view. Examples include stripe arrangement, delta arrangement, PenTile arrangement, and Bayer arrangement. Each subpixel may have any known shape in a plan view. Examples include quadrangles, such as a rectangle and a rhombus, and a hexagon. As a matter of course, a figure that is not strictly rectangular but is close to rectangular is also included in the rectangle. The shape of each subpixel and the pixel array can be used in combination.

### <Applications of Organic Light-Emitting Element>

The organic light-emitting element according to the present embodiment can be used as a constituent of a display apparatus or a lighting apparatus. Other applications include an exposure light source of an electrophotographic image-forming apparatus, a backlight of a liquid crystal display, and a light-emitting apparatus with a color filter in a white light source.

The display apparatus may be an image-information-processing apparatus that includes an image input unit for inputting image information from an area CCD, a linear CCD, a memory card, or the like, includes an information processing unit for processing the input information, and displays an input image on a display unit. The display apparatus may have a plurality of pixels, and at least one of the pixels may include the organic light-emitting element according to the present embodiment and a transistor coupled to the organic light-emitting element.

A display unit of an imaging apparatus or an ink jet printer may have a touch panel function. A driving system of the touch panel function may be, but is not limited to, an infrared radiation system, an electrostatic capacitance system, a resistive film system, or an electromagnetic induction system. The display apparatus may be used for a display unit of a multifunction printer.

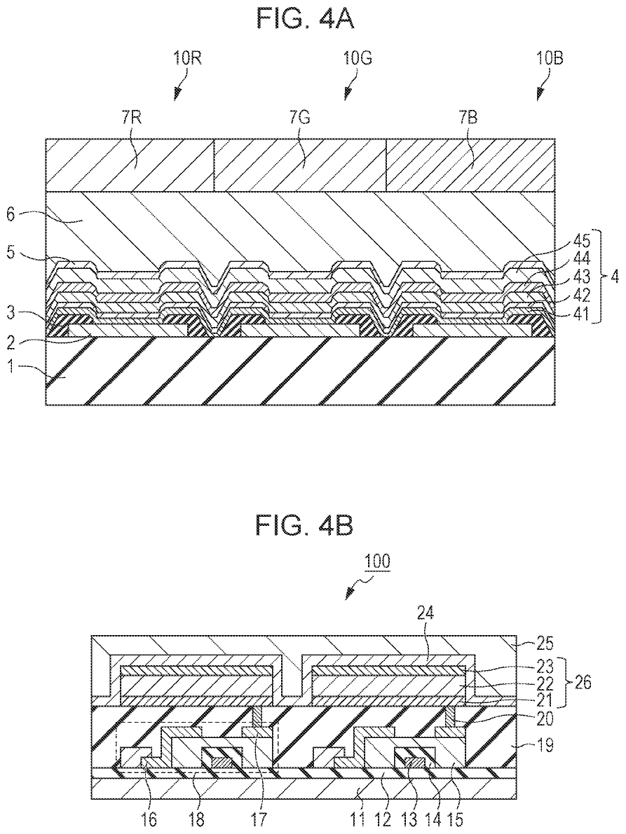

Next, a display apparatus according to the present embodiment is described with reference to the accompanying drawings. FIGS. 4A and 4B are schematic cross-sectional views of an example of a display apparatus that includes an organic light-emitting element and a transistor coupled to the organic light-emitting element. The transistor is an example of an active element. The transistor may be a thin-film transistor (TFT).

FIG. 4A illustrates an example of a pixel serving as a constituent of the display apparatus according to the present embodiment. The pixel has subpixels 10. The subpixels are 10R, 10G, and 10B with different emission colors. The emission colors may be distinguished by the wavelength of light emitted from the light-emitting layer, or light emitted from each subpixel may be selectively transmitted or color-converted with a color filter or the like. Each of the subpixels 10 includes a reflective electrode serving as a first electrode 2, an insulating layer 3 covering an end of the first electrode 2, organic compound layers 4 covering the first electrode 2 and the insulating layer 3, a transparent electrode serving as a second electrode 5, a protective layer 6, and a color filter 7 on an interlayer insulating layer 1.

A transistor and/or a capacitor element may be provided under or inside the interlayer insulating layer 1. The transistor may be electrically connected to the first electrode via a contact hole (not shown) or the like.

The insulating layer 3 is also referred to as a bank or a pixel separation film. The insulating layer 3 covers the ends of the first electrode 2 and surrounds the first electrode 2. A portion not covered with the insulating layer 3 is in contact with the organic compound layers 4 and serves as a light-emitting region.

The organic compound layers 4 include a hole-injection layer 41, a hole-transport layer 42, a first light-emitting layer 43, a second light-emitting layer 44, and an electron-transport layer 45.

The second electrode 5 may be a transparent electrode, a reflective electrode, or a semitransparent electrode.

The protective layer 6 reduces the penetration of moisture into the organic compound layers 4. The protective layer 6 is illustrated as a single layer but may be a plurality of layers. The protective layer may include an inorganic compound layer and an organic compound layer.

The color filter 7 is divided into 7R, 7G, and 7B according to the color. The color filter 7 may be formed on a planarization film (not shown). Furthermore, a resin protective layer (not shown) may be provided on the color filter 7. The color filter 7 may be formed on the protective layer 6. Alternatively, the color filter 7 may be bonded after being provided on an opposite substrate, such as a glass substrate.

A display apparatus 100 in FIG. 4B includes an organic light-emitting element 26 and a TFT 18 as an example of a transistor. The display apparatus 100 includes a substrate 11 made of glass, silicon, or the like and an insulating layer 12 on the substrate 11. An active element, such as the TFT 18, and a gate electrode 13, a gate-insulating film 14, and a semiconductor layer 15 of the active element are provided on the insulating layer 12. The TFT 18 is also composed of a drain electrode 16 and a source electrode 17. The TFT 18 is covered with an insulating film 19. A positive electrode 21 of the organic light-emitting element 26 is coupled to the source electrode 17 through a contact hole 20 formed in the insulating film 19.

Electrical connection between the electrodes of the organic light-emitting element 26 (the positive electrode 21 and a negative electrode 23) and the electrodes of the TFT 18 (the source electrode 17 and the drain electrode 16) is not limited to that illustrated in FIG. 4B. More specifically, it is only necessary to electrically connect either the positive electrode 21 or the negative electrode 23 to either the source electrode 17 or the drain electrode 16 of the TFT 18. TFT refers to a thin-film transistor.

Although an organic compound layer 22 is a single layer in the display apparatus 100 illustrated in FIG. 4B, the organic compound layer 22 may be composed of a plurality of layers. The negative electrode 23 is covered with a first protective layer 24 and a second protective layer 25 for preventing degradation of the organic light-emitting element 26.

The transistor used as a switching element in the display apparatus 100 illustrated in FIG. 4B may be replaced with another switching element.

The transistor used in the display apparatus 100 in FIG. 4B is not limited to a transistor including a single crystal silicon wafer and may also be a thin-film transistor including an active layer on an insulating surface of a substrate. The active layer may be single-crystal silicon, non-single-crystal silicon, such as amorphous silicon or microcrystalline silicon, or a non-single-crystal oxide semiconductor, such as indium zinc oxide or indium gallium zinc oxide. The thin-film transistor is also referred to as a TFT element.

The transistor in the display apparatus 100 of FIG. 4B may be formed within a substrate, such as a Si substrate. The phrase “formed within a substrate” means that the substrate, such as a Si substrate, itself is processed to form the transistor. Thus, the transistor within the substrate can be considered that the substrate and the transistor are integrally formed.

In the organic light-emitting element according to the present embodiment, the luminous brightness is controlled with the TFT, which is an example of a switching element. The organic light-emitting element can be provided in a plurality of planes to display an image at each luminous brightness. The switching element according to the present embodiment is not limited to the TFT and may be a transistor formed of low-temperature polysilicon or an active-matrix driver formed on a substrate, such as a Si substrate. “On a substrate” may also be referred to as “within a substrate”. Whether a transistor is provided within a substrate or a TFT is used depends on the size of a display unit. For example, for an approximately 0.5-inch display unit, an organic light-emitting element can be provided on a Si substrate.

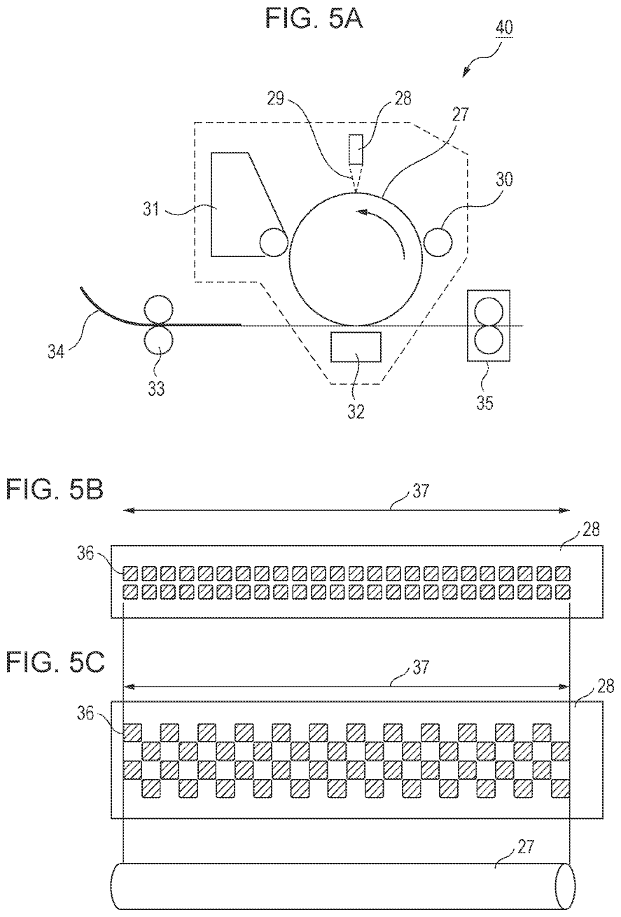

FIG. 5A is a schematic view of an example of an image-forming apparatus according to an embodiment of the present disclosure. An image-forming apparatus 40 is an electrophotographic image-forming apparatus and includes a photosensitive unit 27, an exposure light source 28, a charging unit 30, a developing unit 31, a transfer unit 32, a conveying roller 33, and a fixing unit 35. The exposure light source 28 emits light 29 to form an electrostatic latent image on the surface of the photosensitive unit 27 (on the photosensitive unit). The exposure light source 28 includes the organic light-emitting element according to the present embodiment. The developing unit 31 contains toner and the like. The charging unit 30 electrifies the photosensitive unit 27. The transfer unit 32 transfers a developed image onto a recording medium 34. The conveying roller 33 conveys the recording medium 34. The recording medium 34 is paper, for example. The fixing unit 35 fixes an image on the recording medium 34.

FIGS. 5B and 5C are schematic views of the exposure light source 28, wherein a plurality of light-emitting portions 36 are the organic light-emitting elements according to the present embodiment arranged on a long substrate. An arrow 37 indicates a direction parallel to the shaft of the photosensitive unit 27 and indicates a longitudinal direction in which the light-emitting portions 36 are arranged. The longitudinal direction is the same as the direction of the rotation axis of the photosensitive unit 27. This direction can also be referred to as the major-axis direction of the photosensitive unit 27. In FIG. 5B, the light-emitting portions 36 are arranged in the major-axis direction of the photosensitive unit 27. In FIG. 5C, unlike FIG. 5B, the light-emitting portions 36 are alternately arranged in the longitudinal direction in the first and second rows.

The first row and the second row are arranged at different positions in the transverse direction. In the first row, the light-emitting portions 36 are arranged at intervals. In the second row, the light-emitting portions 36 are arranged at positions corresponding to the spaces between the light-emitting portions 36 of the first row. Thus, the light-emitting portions 36 are also arranged at intervals in the transverse direction. The arrangement in FIG. 5C can also be referred to as a grid-like pattern, a staggered pattern, or a checkered pattern, for example.

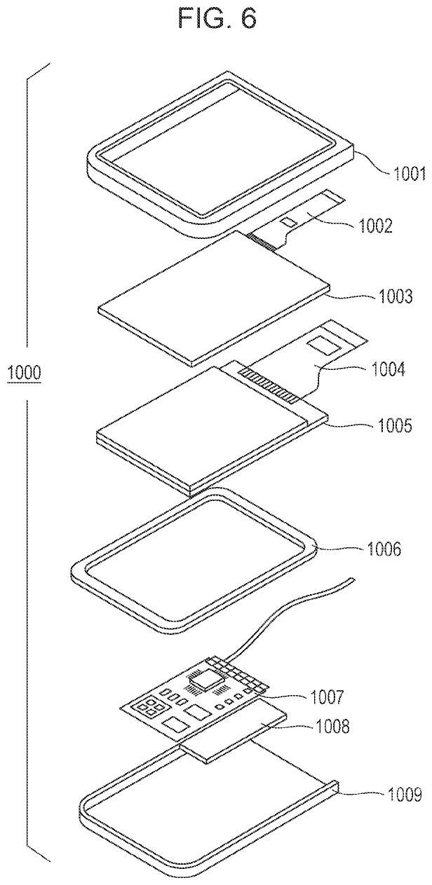

FIG. 6 is a schematic view of an example of the display apparatus according to the present embodiment. A display apparatus 1000 may include a touch panel 1003, a display panel 1005, a frame 1006, a circuit substrate 1007, and a battery 1008 between an upper cover 1001 and a lower cover 1009. The touch panel 1003 and the display panel 1005 are coupled to flexible print circuits FPC 1002 and 1004, respectively. Transistors are printed on the circuit substrate 1007. The battery 1008 may not be provided when the display apparatus is not a mobile device, or may be provided at another position even when the display apparatus is a mobile device.

The display apparatus according to the present embodiment may include color filters of red, green, and blue colors. In the color filters, the red, green, and blue colors may be arranged in a delta arrangement.

The display apparatus according to the present embodiment may be used for a display unit of a mobile terminal. Such a display apparatus may have both a display function and an operation function. Examples of the mobile terminal include mobile phones, such as smartphones, tablets, and head-mounted displays.

The display apparatus according to the present embodiment may be used for a display unit of an imaging apparatus that includes an optical unit with a plurality of lenses and an imaging element for receiving light passing through the optical unit. The imaging apparatus may include a display unit for displaying information acquired by the imaging element. The display unit may be a display unit exposed outside from the imaging apparatus or a display unit located in a finder. The imaging apparatus may be a digital camera or a digital video camera.

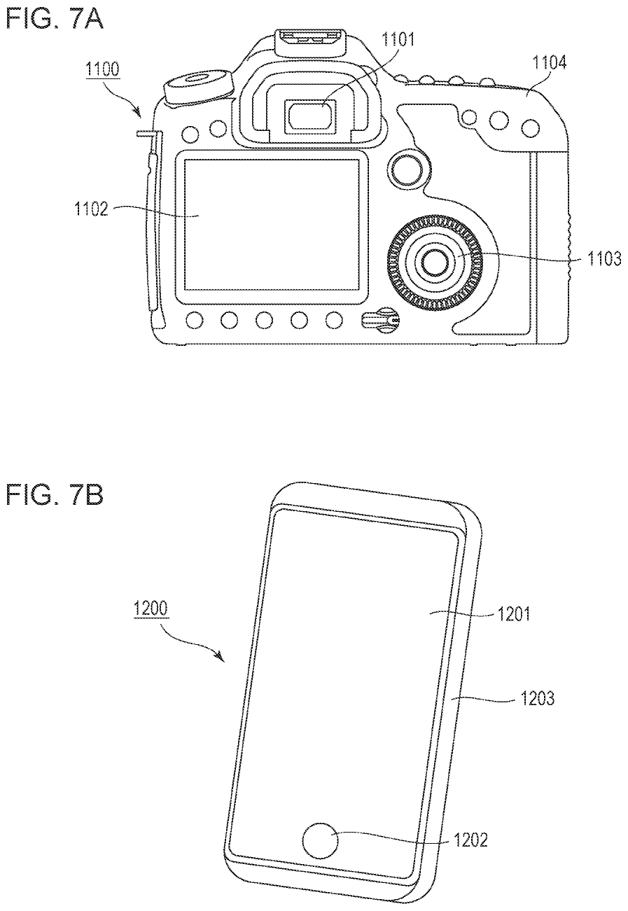

FIG. 7A is a schematic view of an example of an imaging apparatus according to the present embodiment. An imaging apparatus 1100 may include a viewfinder 1101, a rear display 1102, an operating unit 1103, and a housing 1104. The viewfinder 1101 may include the display apparatus according to the present embodiment. In such a case, the display apparatus may display environmental information, imaging instructions, and the like as well as an image to be captured. The environmental information may include the intensity of external light, the direction of external light, the travel speed of the photographic subject, the possibility that the photographic subject is shielded by a shielding material, and the like.

Because the appropriate timing for imaging is a short time, it is better to display information as soon as possible. Thus, a display apparatus including the organic light-emitting element according to the present embodiment can be used. This is because the organic light-emitting element has a high response speed. A display apparatus including the organic light-emitting element can be more suitably used than these apparatuses and liquid crystal displays that require a high display speed.

The imaging apparatus 1100 includes an optical unit (not shown). The optical unit has a plurality of lenses and focuses an image on an imaging element in the housing 1104. The focus of the lenses can be adjusted by adjusting their relative positions. This operation can also be automatically performed. The imaging apparatus may also be referred to as a photoelectric conversion apparatus. The photoelectric conversion apparatus can have, as an imaging method, a method of detecting a difference from a previous image or a method of cutting out a permanently recorded image, instead of taking an image one after another.

FIG. 7B is a schematic view of an example of electronic equipment according to the present embodiment. Electronic equipment 1200 includes a display unit 1201, an operating unit 1202, and a housing 1203. The housing 1203 may include a circuit, a printed circuit board including the circuit, a battery, and a communication unit. The operating unit 1202 may be a button or a touch panel response unit. The operating unit 1202 may be a biometric recognition unit that recognizes a fingerprint and releases the lock. Electronic equipment with a communication unit may also be referred to as communication equipment. The electronic equipment 1200 may have a lens and an imaging element and thereby further have a camera function. An image captured by the camera function is displayed on the display unit 1201. The electronic equipment 1200 may be a smartphone, a notebook computer, or the like.

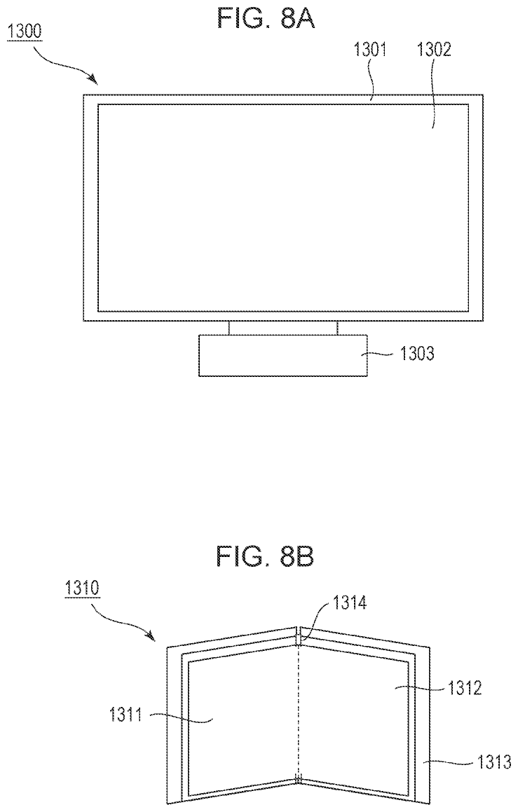

FIGS. 8A and 8B are schematic views of an example of the display apparatus according to the present embodiment. FIG. 8A illustrates a display apparatus, such as a television monitor or a PC monitor. A display apparatus 1300 includes a frame 1301 and a display unit 1302. The light-emitting element according to the present embodiment may be used for the display unit 1302. The frame 1301 and the display unit 1302 are supported by a base 1303. The base 1303 is not limited to the structure illustrated in FIG. 8A. The lower side of the frame 1301 may also serve as the base. The frame 1301 and the display unit 1302 may be bent. The radius of curvature may range from 5000 to 6000 mm.

FIG. 8B is a schematic view of another example of the display apparatus according to the present embodiment. A display apparatus 1310 in FIG. 8B is configured to be foldable and is a so-called foldable display apparatus. The display apparatus 1310 includes a first display unit 1311, a second display unit 1312, a housing 1313, and a folding point 1314. The first display unit 1311 and the second display unit 1312 may include the light-emitting element according to the present embodiment. The first display unit 1311 and the second display unit 1312 may be a single display apparatus without a joint. The first display unit 1311 and the second display unit 1312 can be divided by a folding point. The first display unit 1311 and the second display unit 1312 may display different images or one image.

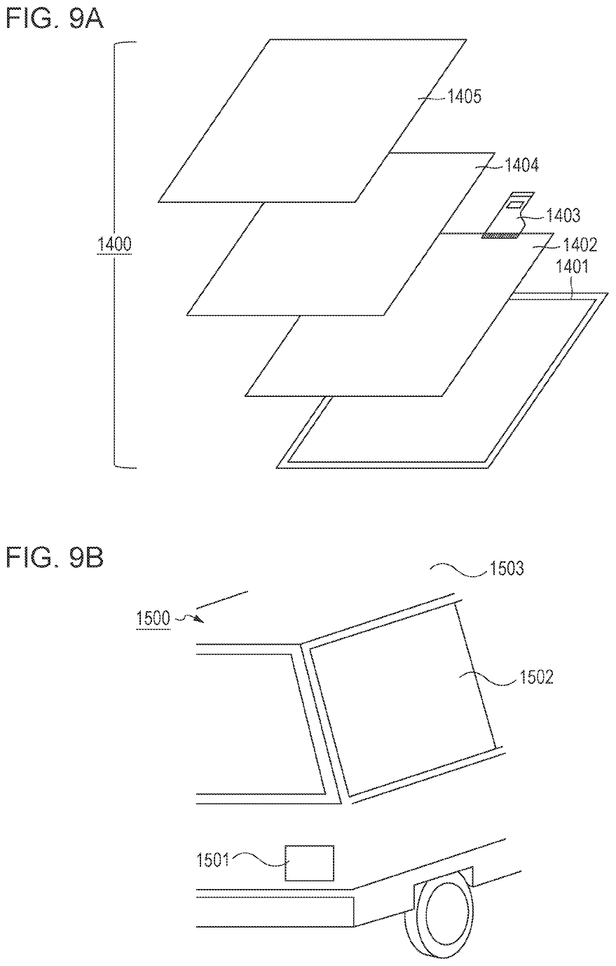

FIG. 9A is a schematic view of an example of a lighting apparatus according to the present embodiment. A lighting apparatus 1400 may include a housing 1401, a light source 1402, a circuit substrate 1403, an optical filter 1404 that transmits light emitted by the light source 1402, and a light-diffusing unit 1405. The light source 1402 may include the organic light-emitting element according to the present embodiment. The optical filter 1404 may be a filter that improves the color rendering properties of the light source. The light-diffusing unit 1405 can effectively diffuse light from the light source and widely spread light as in illumination. The optical filter 1404 and the light-diffusing unit 1405 may be provided on the light output side of the illumination. If necessary, a cover may be provided on the outermost side.

For example, the lighting apparatus is an interior lighting apparatus. The lighting apparatus may emit white light, neutral white light, or light of any color from blue to red. The lighting apparatus may have a light control circuit for controlling such light.

The lighting apparatus may include the organic light-emitting element according to the present embodiment and a power supply circuit coupled thereto. The power supply circuit is a circuit that converts an AC voltage to a DC voltage. White has a color temperature of 4200 K, and neutral white has a color temperature of 5000 K. The lighting apparatus may have a color filter.

The lighting apparatus according to the present embodiment may include a heat dissipation unit. The heat dissipation unit releases heat from the apparatus to the outside and may be a metal or liquid silicon with a high specific heat.

FIG. 9B is a schematic view of an automobile as an example of a moving body according to the present embodiment. The automobile has a taillight as an example of a lamp. An automobile 1500 may have a taillight 1501, which comes on when a brake operation or the like is performed.

The taillight 1501 may include the organic light-emitting element according to the present embodiment. The taillight 1501 may include a protective member for protecting the organic light-emitting element. The protective member may be formed of any transparent material with moderately high strength and can be formed of polycarbonate or the like. The polycarbonate may be mixed with a furan dicarboxylic acid derivative, an acrylonitrile derivative, or the like.

The automobile 1500 may have a body 1503 and a window 1502 on the body 1503. The window 1502 may be a transparent display as long as it is not a window for checking the front and rear of the automobile. The transparent display may include the organic light-emitting element according to the present embodiment. In such a case, constituent materials, such as electrodes, of the organic light-emitting element are transparent materials.

The moving body according to the present embodiment may be a ship, an aircraft, a drone, or the like. The moving body may include a body and a lamp provided on the body. The lamp may emit light to indicate the position of the body. The lamp includes the organic light-emitting element according to the present embodiment.

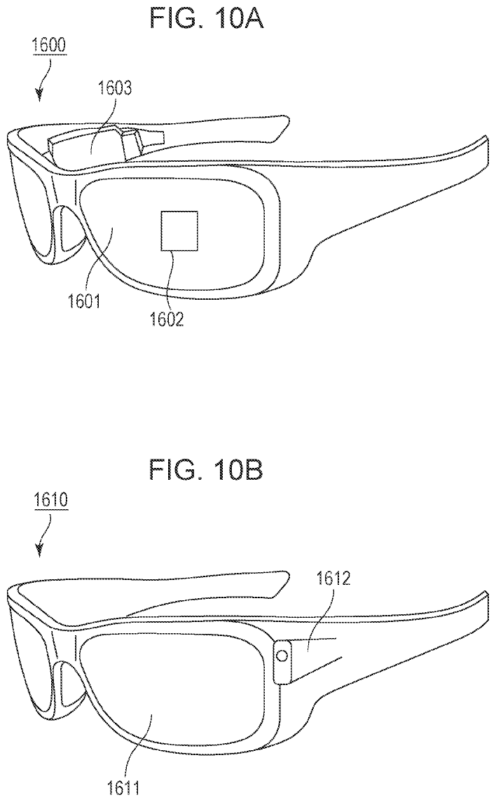

Application examples of the display apparatus according to each of the embodiments are described below with reference to FIGS. 10A and 10B. The display apparatus can be applied to a system that can be worn as a wearable device, such as smart glasses, a head-mounted display (HMD), or smart contact lenses. An imaging and displaying apparatus used in such an application example includes an imaging apparatus that can photoelectrically convert visible light and a display apparatus that can emit visible light.

FIG. 10A is a schematic view of an example of a wearable device according to an embodiment of the present disclosure. Glasses 1600 (smart glasses) according to one application example are described below with reference to FIG. 10A. An imaging apparatus 1602, such as a complementary metal-oxide semiconductor (CMOS) sensor or a single-photon avalanche photodiode (SPAD), is provided on the front side of a lens 1601 of the glasses 1600. The display apparatus according to one of the embodiments is provided on the back side of the lens 1601.

The glasses 1600 further include a controller 1603. The controller 1603 functions as a power supply for supplying power to the imaging apparatus 1602 and the display apparatus. The controller 1603 controls the operation of the imaging apparatus 1602 and the display apparatus. The lens 1601 has an optical system for focusing light on the imaging apparatus 1602.

FIG. 10B is a schematic view of another example of a wearable device according to an embodiment of the present disclosure. Glasses 1610 (smart glasses) according to one application example are described below with reference to FIG. 10B. The glasses 1610 have a controller 1612, which includes an imaging apparatus corresponding to the imaging apparatus 1602 of FIG. 10A and a display apparatus. A lens 1611 includes an optical system for projecting light from the imaging apparatus of the controller 1612 and the display apparatus, and an image is projected on the lens 1611. The controller 1612 functions as a power supply for supplying power to the imaging apparatus and the display apparatus and controls the operation of the imaging apparatus and the display apparatus.

The controller 1612 may include a line-of-sight detection unit for detecting the line of sight of the wearer. Infrared radiation may be used to detect the line of sight. An infrared radiation unit emits infrared light to an eyeball of a user who is gazing at a display image. Reflected infrared light from the eyeball is detected by an imaging unit including a light-receiving element to capture an image of the eyeball. A reduction unit for reducing light from the infrared radiation unit to a display unit in a plan view is provided to reduce degradation in image quality. The line of sight of the user for the display image is detected from the image of the eyeball captured by infrared imaging. Any known technique can be applied to line-of-sight detection using the image of the eyeball. For example, it is possible to use a line-of-sight detection method based on a Purkinje image obtained by reflection of irradiation light by the cornea. More specifically, a line-of-sight detection process based on a pupil-corneal reflection method is performed. The line of sight of the user is detected by calculating a line-of-sight vector representing the direction (rotation angle) of an eyeball on the basis of an image of a pupil and a Purkinje image included in a captured image of the eyeball using the pupil-corneal reflection method.

A display apparatus according to an embodiment of the present disclosure may include an imaging apparatus including a light-receiving element and may control a display image on the basis of line-of-sight information of a user from the imaging apparatus. More specifically, on the basis of the line-of-sight information, the display apparatus determines a first visibility region at which the user gazes and a second visibility region other than the first visibility region. The first visibility region and the second visibility region may be determined by the controller of the display apparatus or may be received from an external controller. In the display region of the display apparatus, the first visibility region may be controlled to have higher display resolution than the second visibility region. In other words, the second visibility region may have lower resolution than the first visibility region.

The display region has a first display region and a second display region different from the first display region, and the priority of the first display region and the second display region depends on the line-of-sight information. The first visibility region and the second visibility region may be determined by the controller of the display apparatus or may be received from an external controller. A region with a higher priority may be controlled to have higher resolution than another region. In other words, a region with a lower priority may have lower resolution.

The first visibility region or a region with a higher priority may be determined by artificial intelligence (AI). The A1 may be a model configured to estimate the angle of the line of sight and the distance to a target ahead of the line of sight from an image of an eyeball using the image of the eyeball and the direction in which the eyeball actually viewed in the image as teaching data. The A1 program may be stored in the display apparatus, the imaging apparatus, or an external device. The A1 program stored in an external device is transmitted to the display apparatus via communication.

For display control based on visual recognition detection, the present disclosure can be applied to smart glasses further having an imaging apparatus for imaging the outside. Smart glasses can display captured external information in real time.

As described above, an apparatus including the organic light-emitting element according to the present embodiment can be used to stably display a high-quality image for extended periods.

### EXAMPLES

The present disclosure is described below with exemplary embodiments. However, the present disclosure is not limited these exemplary embodiments.

### Exemplary Embodiment 1 (Synthesis of Exemplary Compound A4)

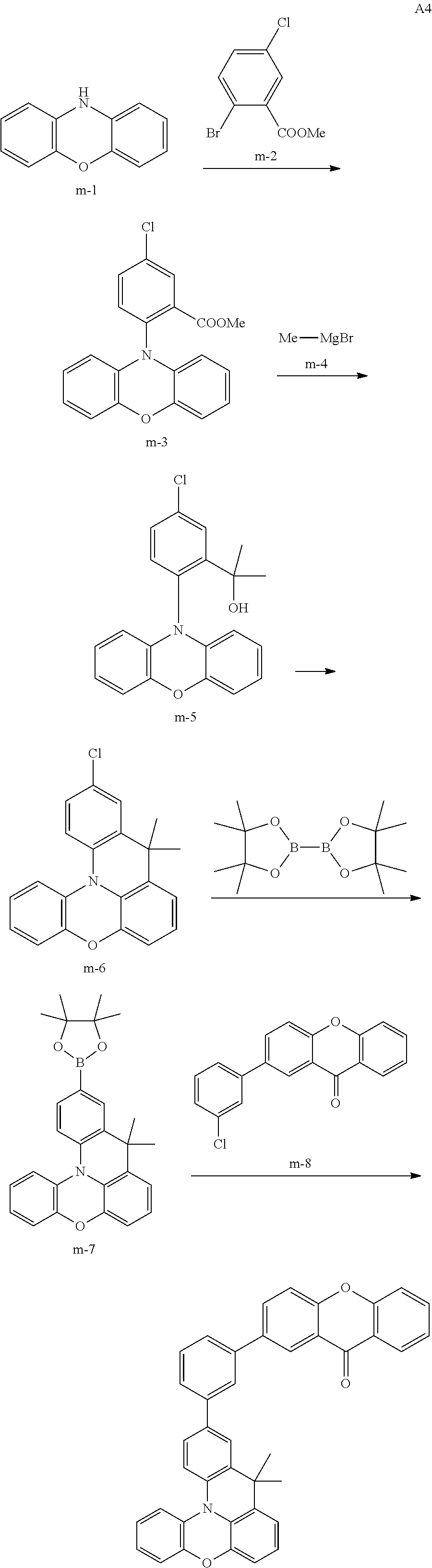

### (1) Synthesis of Compound m-3

A 200-ml recovery flask was charged with the following reagents and solvent.

Compound m-1: 5.0 g (27.3 mmol)
Compound m-2: 7.5 g (30.0 mmol)
Copper: 1.7 g (27.3 mmol)
Potassium carbonate: 3.8 g (27.3 mmol)
Sodium sulfate: 3.9 g (27.3 mmol)

### Nitrobenzene: 100 ml

The reaction solution was then heated with stirring at 220° C. for 7 hours under nitrogen. After completion of the reaction, the reaction solution was concentrated under reduced pressure, and ethyl acetate was added to the resulting residue. The residue was washed with aqueous ammonium chloride, and the organic layer was concentrated under reduced pressure to give a crude product. The crude product was then purified by silica gel column chromatography (chloroform) to give 7.5 g (yield: 78%) of a compound m-3 as a white solid.

### (2) Synthesis of Compound m-5

A 200-ml recovery flask was charged with the following reagent and solvent.

Compound m-3: 5.0 g (14.2 mmol)
Dehydrated tetrahydrofuran: 150 ml

Next, 42.6 ml (42.6 mmol) of a 1.0 M methyl magnesium bromide solution (tetrahydrofuran) represented by m-4 was added dropwise to the reaction solution under nitrogen at an internal temperature of 0° C. The reaction solution was then heated to room temperature and was stirred at this temperature (room temperature) for 5 hours. After completion of the reaction, toluene was added to the reaction solution, and the reaction solution was washed with saturated saline. The organic layer was concentrated under reduced pressure to give a crude product. The crude product was then purified by silica gel column chromatography (chloroform) to give 4.1 g (yield: 82%) of a compound m-5.

### (3) Synthesis of Compound m-6

A 200-ml recovery flask was charged with the following reagents and solvent.

Compound m-5: 4.0 g (11.4 mmol)
Polyphosphoric acid: 35 ml

The reaction solution was then heated with stirring at 205° C. for 2 hours under nitrogen. After completion of the reaction, the reaction solution was allowed to cool. Toluene was then added to the reaction solution, and the reaction solution was neutralized and washed with aqueous sodium carbonate. The organic layer was then concentrated under reduced pressure to give a crude product. The crude product was then purified by column chromatography (NH gel) to give 1.0 g (yield: 27%) of a compound m-6.

### (4) Synthesis of Compound m-7

A 200-ml recovery flask was charged with the following reagents and solvent.

Compound m-6: 1.0 g (3.0 mmol)
Bis(pinacolato)diboron: 0.9 g (3.6 mmol)
Bis(dibenzylideneacetone)palladium(0): 0.3 g (0.6 mmol)
2-dicyclohexylphosphino-2′,6′-dimethoxybiphenyl (S-Phos): 0.5 g (1.2 mmol) Potassium acetate: 0.9 g (9.0 mmol) Dehydrated 1,4-dioxane: 50 ml

The reaction solution was then heated with stirring at 125° C. for 7 hours under nitrogen. After completion of the reaction, the reaction solution was allowed to cool. Toluene was then added to the reaction solution, and the reaction solution was washed with saturated saline. The organic layer was then concentrated to give a crude product. The crude product was then purified by column chromatography (chloroform) to give 0.9 g (yield: 72%) of a compound m-7.

### (3) Synthesis of Compound A4

A 200-ml recovery flask was charged with the following reagents and solvents.

Compound m-7: 0.9 g (2.1 mmol)
Compound m-8: 0.8 g (2.5 mmol)
Bis(dibenzylideneacetone)palladium(0): 0.2 g (0.4 mmol)
2-dicyclohexylphosphino-2′,6′-dimethoxybiphenyl (S-Phos): 0.4 g (0.8 mmol) Potassium phosphate: 1.4 g (6.3 mmol)

### Toluene: 50 ml

### H₂O: 5 ml

The reaction solution was then heated and stirred under reflux in a nitrogen stream for 6 hours. After completion of the reaction, water was added to the product for separation. The product was dissolved in chloroform, was purified by column chromatography (chloroform:heptane), and was recrystallized in toluene/heptane to give 0.8 g (yield: 65%) of an exemplary compound A4 as a pale yellow solid.

The exemplary compound A4 was subjected to mass spectrometry with MALDI-TOF-MS (Autoflex LRF manufactured by Bruker). The measured value (m/z) was 569, which agreed with the value 569 calculated for C₄₀H₂₇NO₃.

### Exemplary Embodiments 2 to 10 (Synthesis of Exemplary Compounds)

Exemplary compounds according to Exemplary Embodiments 2 to 10 in Table 4 were synthesized in the same manner as in Exemplary Embodiment 1 except that the compounds m-1, m-2, and m-8 according to Exemplary Embodiment 1 were changed to the compounds shown in Table 4. Actual values (m/z) measured by mass spectrometry in the same manner as in Exemplary Embodiment 1 are also shown.

TABLE 4

| Exemplary | Exemplary | Raw material | Raw material | Raw material | |
|---|---|---|---|---|---|
| embodiment | compound | m-1 | m-2 | m-8 | m/z |
| 2 | A2 | 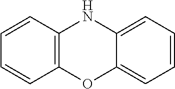 [SMILES: c1ccc2c(c1)Nc1ccccc1O2] | 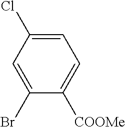 [SMILES: COC(=O)c1ccc(Cl)cc1Br] | 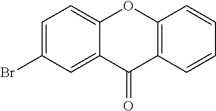 [SMILES: O=c1c2ccccc2oc2ccc(Br)cc12] | 493 |
| 3 | A7 | 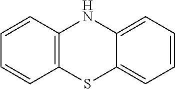 [SMILES: c1ccc2c(c1)Nc1ccccc1S2] | 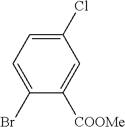 [SMILES: COC(=O)c1cc(Cl)ccc1Br] | 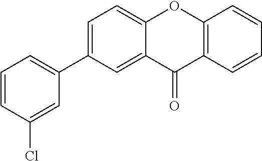 [SMILES: O=c1c2ccccc2oc2ccc(-c3cccc(Cl)c3)cc12] | 585 |
| 4 | A9 | 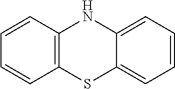 [SMILES: c1ccc2c(c1)Nc1ccccc1S2] | 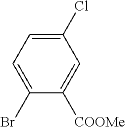 [SMILES: COC(=O)c1cc(Cl)ccc1Br] | 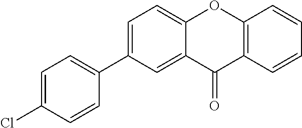 [SMILES: O=c1c2ccccc2oc2ccc(-c3ccc(Cl)cc3)cc12] | 585 |
| 5 | A27 | 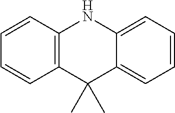 [SMILES: CC1(C)c2ccccc2Nc2ccccc21] | 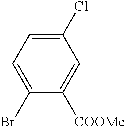 [SMILES: COC(=O)c1cc(Cl)ccc1Br] | 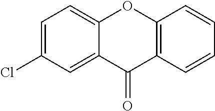 [SMILES: O=c1c2ccccc2oc2ccc(Cl)cc12] | 519 |
| 6 | A10 | 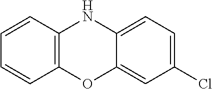 [SMILES: Clc1ccc2c(c1)Oc1ccccc1N2] | 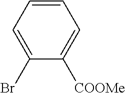 [SMILES: COC(=O)c1ccccc1Br] | 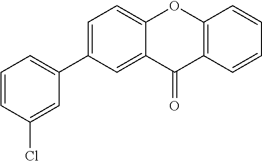 [SMILES: O=c1c2ccccc2oc2ccc(-c3cccc(Cl)c3)cc12] | 569 |
| 7 | A11 | 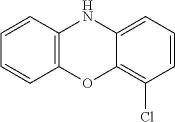 [SMILES: Clc1cccc2c1Oc1ccccc1N2] | 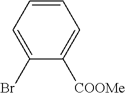 [SMILES: COC(=O)c1ccccc1Br] | 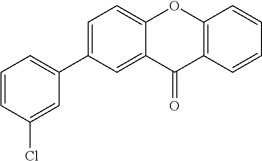 [SMILES: O=c1c2ccccc2oc2ccc(-c3cccc(Cl)c3)cc12] | 569 |
| 8 | A19 | 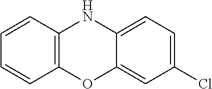 [SMILES: Clc1ccc2c(c1)Oc1ccccc1N2] | 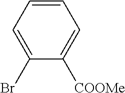 [SMILES: COC(=O)c1ccccc1Br] | 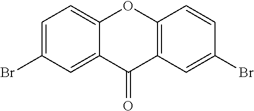 [SMILES: O=c1c2cc(Br)ccc2oc2ccc(Br)cc12] | 790 |
| 9 | A37 | 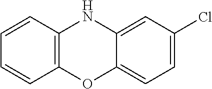 [SMILES: Clc1ccc2c(c1)Nc1ccccc1O2] | 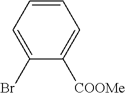 [SMILES: COC(=O)c1ccccc1Br] | 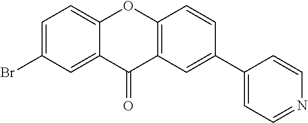 [SMILES: O=c1c2cc(Br)ccc2oc2ccc(-c3ccncc3)cc12] | 570 |
| 10 | A22 | 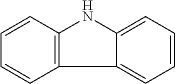 [SMILES: c1ccc2c(c1)[nH]c1ccccc12] | 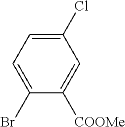 [SMILES: COC(=O)c1cc(Cl)ccc1Br] | 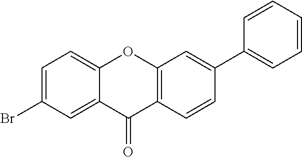 [SMILES: O=c1c2ccc(-c3ccccc3)cc2oc2ccc(Br)cc12] | 553 |

### Exemplary Embodiment 11 (Synthesis of Exemplary Compound B6)

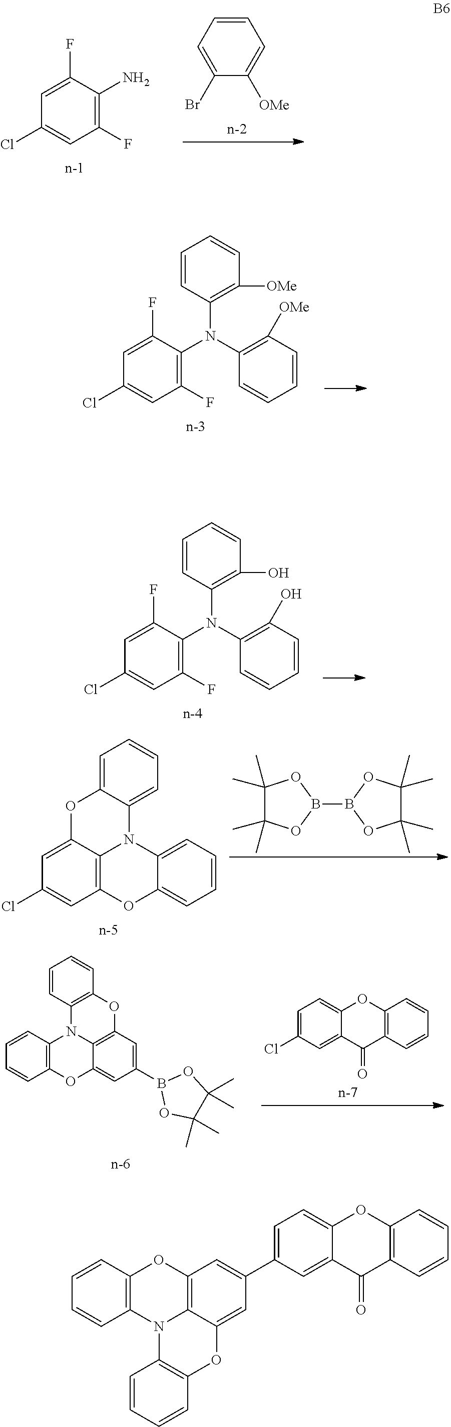

### (1) Synthesis of Compound n-3

A 500-ml recovery flask was charged with the following reagents and solvent.

Compound n-1: 10.0 g (61.1 mmol)
Compound n-2: 34.3 g (183.4 mmol)
Copper: 11.6 g (183.4 mmol)
Potassium carbonate: 25.4 g (183.4 mmol)
Sodium sulfate: 8.61 g (183.4 mmol)

### Nitrobenzene: 200 ml

The reaction solution was then heated with stirring at 220° C. for 7 hours under nitrogen. After completion of the reaction, the reaction solution was concentrated under reduced pressure, and ethyl acetate was added to the resulting residue. The residue was washed with aqueous ammonium chloride, and the organic layer was concentrated under reduced pressure to give a crude product. The crude product was then purified by column chromatography (chloroform) to give 16.8 g (yield: 73%) of a compound n-3 as a brownish white solid.

### (2) Synthesis of Compound n-4

A 200-ml recovery flask was charged with the following reagent and solvent.

Compound n-3: 15.0 g (39.9 mmol)
Pyridine hydrochloric acid: 220 g

The reaction solution was then heated and stirred under reflux in a nitrogen stream for 6 hours. After completion of the reaction, water was added to the product for separation. The product was dissolved in chloroform, was purified by column chromatography (chloroform:heptane), and was recrystallized in toluene/heptane to give 11.2 g (yield: 81%) of a compound n-4 as a white solid.

### (3) Synthesis of Compound n-5

A 500-ml recovery flask was charged with the following reagents and solvent.

Compound n-4: 10.0 g (28.8 mol)
Potassium carbonate: 11.9 g (86.3 mmol)

### N,N-dimethylformamide: 300 ml

The reaction solution was then heated under reflux with stirring for 7 hours under nitrogen. After completion of the reaction, the reaction solution was concentrated under reduced pressure, and ethyl acetate was added to the resulting residue. The residue was washed with aqueous ammonium chloride, and the organic layer was concentrated under reduced pressure to give a crude product. The crude product was then purified by column chromatography (chloroform) to give 6.0 g (yield: 68%) of a compound n-5 as a white solid.

### (4) Synthesis of Compound n-6

A 500-ml recovery flask was charged with the following reagents and solvent.

Compound n-5: 5.0 g (16.2 mmol)
Bis(pinacolato)diboron: 4.9 g (19.5 mmol)
Bis(dibenzylideneacetone)palladium(0): 1.8 g (3.2 mmol)
2-dicyclohexylphosphino-2′,6′-dimethoxybiphenyl (S-Phos): 2.7 g (6.5 mmol)
Potassium acetate: 4.8 g (48.7 mmol)
Dehydrated 1,4-dioxane: 250 ml

The reaction solution was then heated with stirring at 125° C. for 7 hours under nitrogen. After completion of the reaction, the reaction solution was allowed to cool. Toluene was then added to the reaction solution, and the reaction solution was washed with saturated saline. The organic layer was then concentrated to give a crude product. The crude product was then purified by column chromatography (chloroform) to give 4.5 g (yield: 65%) of a compound n-6.

### (5) Synthesis of Compound B4

A 200-ml recovery flask was charged with the following reagents and solvents.

Compound n-6: 1.0 g (2.5 mmol)
Compound n-7: 1.0 g (2.5 mmol)
Bis(dibenzylideneacetone)palladium(0): 0.3 g (0.5 mmol)
2-dicyclohexylphosphino-2′,6′-dimethoxybiphenyl (S-Phos): 0.4 g (1.0 mmol)
Potassium phosphate: 1.6 g (0.8 mmol)

### Toluene: 50 ml

### H₂O: 5 ml

The reaction solution was then heated and stirred under reflux in a nitrogen stream for 6 hours. After completion of the reaction, water was added to the product for separation. The product was dissolved in chloroform, was purified by column chromatography (chloroform:heptane), and was recrystallized in toluene/heptane to give 0.7 g (yield: 61%) of an exemplary compound B4 as a pale yellow solid.

The exemplary compound B4 was subjected to mass spectrometry in the same manner as in Exemplary Embodiment 1. The measured value (m/z) was 467, which agreed with the value 467 calculated for C₃₁H₁₇NO₄.

### Exemplary Embodiments 12 to 20 (Synthesis of Exemplary Compounds)

Exemplary compounds according to Exemplary Embodiments 12 to 20 in Table 5 were synthesized in the same manner as in Exemplary Embodiment 11 except that the compounds n-5 and n-7 according to Exemplary Embodiment 11 were changed to the compounds shown in Table 5. Actual values (m/z) measured by mass spectrometry in the same manner as in Exemplary Embodiment 11 are also shown.

TABLE 5

| Exemplary | Exemplary | Raw material | Raw material | |
|---|---|---|---|---|
| embodiment | compound | n-7 | n-8 | m/z |
| 12 | B3 | 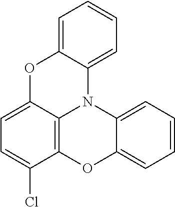 [SMILES: Clc1ccc2c3c1Oc1ccccc1N3c1ccccc1O2] | 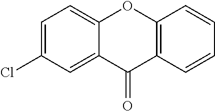 [SMILES: O=c1c2ccccc2oc2ccc(Cl)cc12] | 467 |
| 13 | B1 | 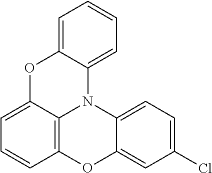 [SMILES: Clc1ccc2c(c1)Oc1cccc3c1N2c1ccccc1O3] | 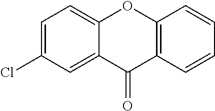 [SMILES: O=c1c2ccccc2oc2ccc(Cl)cc12] | 467 |
| 14 | B2 | 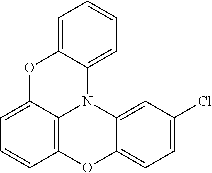 [SMILES: Clc1ccc2c(c1)N1c3ccccc3Oc3cccc(c31)O2] | 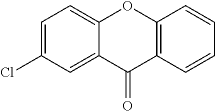 [SMILES: O=c1c2ccccc2oc2ccc(Cl)cc12] | 467 |
| 15 | B10 | 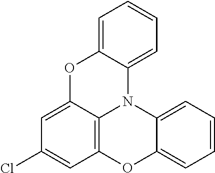 [SMILES: Clc1cc2c3c(c1)Oc1ccccc1N3c1ccccc1O2] | 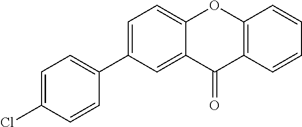 [SMILES: O=c1c2ccccc2oc2ccc(-c3ccc(Cl)cc3)cc12] | 543 |
| 16 | B12 | 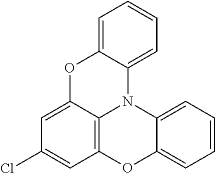 [SMILES: Clc1cc2c3c(c1)Oc1ccccc1N3c1ccccc1O2] | 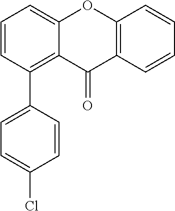 [SMILES: O=c1c2ccccc2oc2cccc(-c3ccc(Cl)cc3)c12] | 543 |
| 17 | B17 | 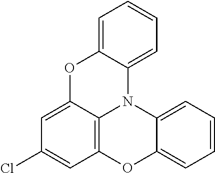 [SMILES: Clc1cc2c3c(c1)Oc1ccccc1N3c1ccccc1O2] | 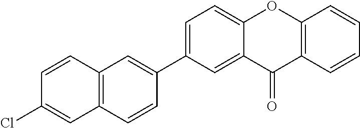 [SMILES: O=c1c2ccccc2oc2ccc(-c3ccc4cc(Cl)ccc4c3)cc12] | 593 |
| 18 | B21 | 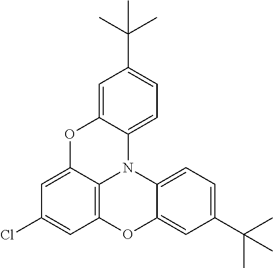 [SMILES: CC(C)(C)c1ccc2c(c1)Oc1cc(Cl)cc3c1N2c1ccc(C(C)(C)C)cc1O3] | 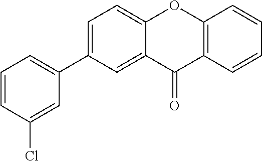 [SMILES: O=c1c2ccccc2oc2ccc(-c3cccc(Cl)c3)cc12] | 655 |
| 19 | B27 | 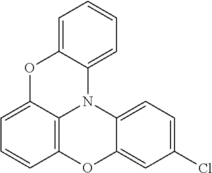 [SMILES: Clc1ccc2c(c1)Oc1cccc3c1N2c1ccccc1O3] | 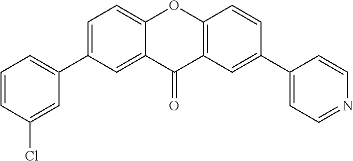 [SMILES: O=c1c2cc(-c3ccncc3)ccc2oc2ccc(-c3cccc(Cl)c3)cc12] | 620 |
| 20 | B13 | 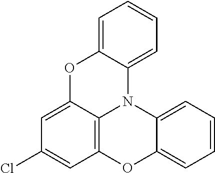 [SMILES: Clc1cc2c3c(c1)Oc1ccccc1N3c1ccccc1O2] | 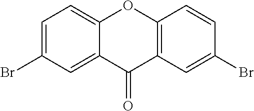 [SMILES: O=c1c2cc(Br)ccc2oc2ccc(Br)cc12] | 738 |

### Comparative Examples 1 and 2 (Synthesis of Comparative Compounds)

Organic compounds of comparative compounds 1 and 2 were synthesized in accordance with the following synthesis scheme. The comparative compound 1 is a compound described in Patent Literature 1, and the comparative compound 2 is a compound similar to a compound described in Patent Literature 2.

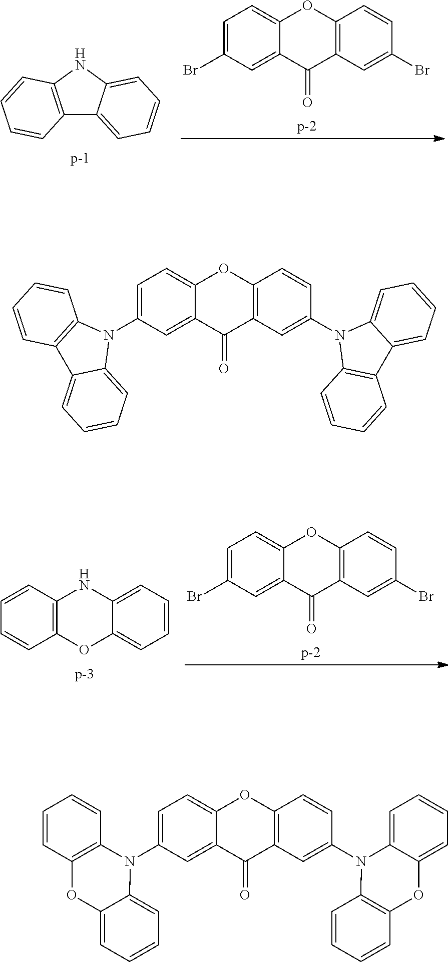

### (1) Synthesis of Comparative Compound 1

A 200-ml recovery flask was charged with the following reagents and solvent.

Compound p-1: 1.0 g (2.8 mmol)
Compound p-2: 1.2 g (7.1 mmol)

### Pd(dba)₂: 0.3 g

xphos: 0.5 g
tBuONa: 0.7 g (7.1 mmol)

### Xylene: 50 ml

The reaction solution was then heated and stirred under reflux in a nitrogen stream for 6 hours. After completion of the reaction, water was added to the product for separation. The product was dissolved in chloroform, was purified by column chromatography (chloroform:heptane), and was recrystallized in toluene/heptane to give 0.7 g (yield: 45%) of the comparative compound 1 as a white solid.

The comparative compound 1 was subjected to mass spectrometry in the same manner as in Exemplary Embodiment 1. The measured value (m/z) was 526, which agreed with the value 526 calculated for C₃₇H₂₂N₂O₂.

### (1) Synthesis of Comparative Compound 2

A 200-ml recovery flask was charged with the following reagents and solvent.

Compound p-3: 1.0 g (2.8 mmol)
Compound p-2: 1.3 g (7.1 mmol)

### Pd(dba)₂: 0.3 g

xphos: 0.5 g
tBuONa: 0.7 g (7.1 mmol)

### Xylene: 50 ml

The reaction solution was then heated and stirred under reflux in a nitrogen stream for 6 hours. After completion of the reaction, water was added to the product for separation. The product was dissolved in chloroform, was purified by column chromatography (chloroform:heptane), and was recrystallized in toluene/heptane to give 0.9 g (yield: 55%) of the comparative compound 2 as a white solid.

The comparative compound 2 was subjected to mass spectrometry in the same manner as in Exemplary Embodiment 1. The measured value (m/z) was 558, which agreed with the value 558 calculated for C₃₇H₂₂N₂O₄.

### Exemplary Embodiment 21

An organic light-emitting element of a bottom emission type was produced. The organic light-emitting element included a positive electrode, a hole-injection layer, a hole-transport layer, an electron-blocking layer, a light-emitting layer, a hole-blocking layer, an electron-transport layer, an electron-injection layer, and a negative electrode sequentially formed on a substrate.

First, an ITO film was formed on a glass substrate and was subjected to desired patterning to form an ITO electrode (positive electrode). The ITO electrode had a thickness of 100 nm. The substrate on which the ITO electrode was formed was used as an ITO substrate in the following process. Vacuum deposition was then performed by resistance heating in a vacuum chamber at 1.33×10⁻⁴ Pa to continuously form an organic compound layer and an electrode layer shown in Table 6 on the ITO substrate. The counter electrode (a metal electrode layer, a negative electrode) had an electrode area of 3 mm².

TABLE 6

| | | | | Film |
|---|---|---|---|---|
| | | | | thickness |
| | Material | | | (nm) |
| Negative electrode | Al | | | 100 |
| Electron-injection layer (EIL) | LiF | | | 1 |
| Electron-transport layer (ETL) | ET2 | | | 20 |
| Hole-blocking layer (HBL) | ET11 | | | 20 |
| Light-emitting layer (EML) | Host | A4 | Weight ratio | 20 |
| | Guest | AA11 | A4:AA11 = | |
| | | | 90:10 | |

| | | |
|---|---|---|
| Electron-blocking layer (EBL) | HT19 | 15 |
| Hole-transport layer (HTL) | HT3 | 30 |
| hole-injection layer (HIL) | HT16 | 5 |

Characteristics of the organic light-emitting element were measured and evaluated. The light-emitting element had a maximum emission wavelength of 522 nm and a maximum external quantum efficiency (E.Q.E.) of 13%. A continuous operation test was performed at a current density of 100 mA/cm² to measure the time (LT95) when the luminance degradation rate reached 5%. When the LT95 of Comparative Example 1 was set to 1.0, the luminance degradation rate ratio of the present exemplary embodiment (LT95 of Exemplary Embodiment 21/LT95 of Comparative Example 1) was 1.4. With respect to measuring apparatuses, the current-voltage characteristics were measured with a microammeter “4140B” manufactured by Hewlett-Packard Co., and the luminous brightness was measured with “BM7” manufactured by Topcon Corporation.

### Exemplary Embodiments 22 to 38, Comparative Examples 3 and 4

Organic light-emitting elements were produced in the same manner as in Exemplary Embodiment 21 except that the compounds shown in Table 7 were used. Characteristics of the elements were measured and evaluated in the same manner as in Exemplary Embodiment 21. Table 7 shows the results.

TABLE 7

| | | | | | | | | | Luminance |
|---|---|---|---|---|---|---|---|---|---|
| | | | | EML | | | | E.Q.E | degradation |
| | HIL | HTL | EBL | Host | Guest | HBL | ETL | [%] | rate ratio |
| Exemplary | HT16 | HT3 | HT19 | A1 | AA26 | ET12 | ET15 | 13 | 1.5 |
| Embodiment 22 | | | | | | | | | |
| Exemplary | HT16 | HT2 | HT15 | A4 | AA27 | ET12 | ET2 | 14 | 1.4 |
| Embodiment 23 | | | | | | | | | |
| Exemplary | HT16 | HT6 | HT14 | A4 | AA30 | ET17 | ET3 | 15 | 1.3 |
| Embodiment 24 | | | | | | | | | |
| Exemplary | HT16 | HT3 | HT19 | A5 | CC17 | ET11 | ET2 | 12 | 1.2 |
| Embodiment 25 | | | | | | | | | |
| Exemplary | HT16 | HT3 | HT19 | A14 | GD10 | ET11 | ET2 | 10 | 1.1 |
| Embodiment 26 | | | | | | | | | |
| Exemplary | HT16 | HT3 | HT19 | A15 | HH1 | ET11 | ET15 | 12 | 1.5 |
| Embodiment 27 | | | | | | | | | |
| Exemplary | HT16 | HT3 | HT19 | B1 | BB26 | ET12 | ET2 | 13 | 1.5 |
| Embodiment 28 | | | | | | | | | |
| Exemplary | HT16 | HT2 | HT15 | B2 | BB25 | ET12 | ET15 | 13 | 1.4 |
| Embodiment 29 | | | | | | | | | |
| Exemplary | HT16 | HT3 | HT19 | B3 | BB27 | ET12 | ET15 | 12 | 1.2 |
| Embodiment 30 | | | | | | | | | |
| Exemplary | HT16 | HT2 | HT15 | B9 | BB29 | ET11 | ET2 | 13 | 1.4 |
| Embodiment 31 | | | | | | | | | |
| Exemplary | HT16 | HT3 | HT19 | B12 | DD31 | ET12 | ET15 | 12 | 1.1 |
| Embodiment 32 | | | | | | | | | |
| Exemplary | HT16 | HT2 | HT15 | B21 | DD27 | ET12 | ET2 | 13 | 1.2 |
| Embodiment 33 | | | | | | | | | |
| Exemplary | HT16 | HT2 | HT15 | B1 | GD10 | ET11 | ET2 | 11 | 1.1 |
| Embodiment 34 | | | | | | | | | |
| Exemplary | HT16 | HT3 | HT19 | B17 | RD7 | ET12 | ET15 | 9 | 1.2 |
| Embodiment 35 | | | | | | | | | |
| Exemplary | HT16 | HT3 | HT19 | A27 | HH19 | ET11 | ET15 | 13 | 1.4 |
| Embodiment 36 | | | | | | | | | |
| Exemplary | HT16 | HT3 | HT19 | B27 | HH21 | ET12 | ET2 | 14 | 1.3 |
| Embodiment 37 | | | | | | | | | |
| Exemplary | HT16 | HT2 | HT15 | B10 | II3 | ET12 | ET15 | 13 | 1.4 |
| Embodiment 38 | | | | | | | | | |
| Comparative | HT16 | HT3 | HT19 | Comparative | GD10 | ET11 | ET2 | 8 | 1.0 |
| Example 3 | | | | compound | | | | | |
| | | | | 1-A | | | | | |
| Comparative | HT16 | HT3 | HT19 | Comparative | GD10 | ET11 | ET2 | 5 | 0.9 |
| Example 4 | | | | compound | | | | | |
| | | | | 1-B | | | | | |

Table 7 shows that the maximum external quantum efficiency (E.Q.E.) of Comparative Examples 3 and 4 was 8% and 5%, respectively, and the light-emitting elements according to the present embodiments had higher light emission efficiency. This is because the exemplary compounds according to the present embodiments had lower T1 energy. Furthermore, the light-emitting elements according to the present embodiments had a longer life. This is because the exemplary compounds according to the present embodiments had better film properties and higher bond stability. Thus, the exemplary compounds according to the present embodiments can be used to provide elements with high efficiency and durability.

### Exemplary Embodiment 39

An organic light-emitting element was produced in the same manner as in Exemplary Embodiment 21 except that the organic compound layer and the electrode layer shown in Table 8 were continuously formed.

TABLE 8

| | | | | Film |
|---|---|---|---|---|
| | | | | thickness |
| | Material | | | (nm) |
| Negative electrode | Al | | | 100 |
| Electron-injection layer (EIL) | LiF | | | 1 |
| Electron-transport layer (ETL) | ET2 | | | 20 |
| Hole-blocking layer (HBL) | ET11 | | | 20 |
| Light-emitting layer (EML) | Host | A4 | Weight ratio | 20 |
| | Guest | AA22 | A4:AA22:EM30 = | |
| | Assist | EM30 | 60:10:30 | |

| | | |
|---|---|---|
| Electron-blocking layer (EBL) | HT19 | 15 |
| Hole-transport layer (HTL) | HT3 | 30 |
| Hole-injection layer (HIL) | HT16 | 5 |

Characteristics of the organic light-emitting element were measured and evaluated in the same manner as in Exemplary Embodiment 21. The light-emitting element had a green emission color and a maximum external quantum efficiency (E.Q.E.) of 18%. In a continuous operation test performed in the same manner as in Exemplary Embodiment 21, the luminance degradation rate ratio of Exemplary Embodiment 39 was 2.5 when the LT95 of Comparative Example 3 was set to 1.0.

### Exemplary Embodiments 40 to 63

Organic light-emitting elements were produced in the same manner as in Exemplary Embodiment 39 except that the compounds shown in Table 9 were used. Characteristics of the light-emitting elements were measured and evaluated in the same manner as in Exemplary Embodiment 39. Table 9 shows the results.

TABLE 9

| | | | | EML | | | | | E.Q.E |
|---|---|---|---|---|---|---|---|---|---|
| | HIL | HTL | EBL | Host | Guest | Assist | HBL | ETL | [%] |
| Exemplary | HT16 | HT3 | HT19 | B11 | BB24 | EM29 | ET26 | ET3 | 17 |
| Embodiment 39 | | | | | | | | | |
| Exemplary | HT16 | HT2 | HT15 | A23 | BB16 | EM35 | ET13 | ET2 | 16 |
| Embodiment 40 | | | | | | | | | |
| Exemplary | HT16 | HT2 | HT15 | B5 | BB25 | EM37 | ET13 | ET2 | 17 |
| Embodiment 41 | | | | | | | | | |
| Exemplary | HT16 | HT3 | HT19 | A28 | DD5 | EM13 | ET16 | ET15 | 15 |
| Embodiment 42 | | | | | | | | | |
| Exemplary | HT16 | HT3 | HT19 | A48 | DD31 | EM30 | ET16 | ET15 | 18 |
| Embodiment 43 | | | | | | | | | |
| Exemplary | HT16 | HT3 | HT19 | B19 | DD27 | B6 | ET17 | ET15 | 15 |
| Embodiment 44 | | | | | | | | | |
| Exemplary | HT16 | HT3 | HT19 | A32 | EE1 | EM39 | ET13 | ET2 | 15 |
| Embodiment 45 | | | | | | | | | |
| Exemplary | HT16 | HT2 | HT15 | A8 | EE2 | GD10 | ET15 | ET3 | 14 |
| Embodiment 46 | | | | | | | | | |
| Exemplary | HT16 | HT3 | HT19 | A5 | FF2 | ET15 | ET15 | ET15 | 16 |
| Embodiment 47 | | | | | | | | | |
| Exemplary | HT16 | HT2 | HT15 | A4 | FF23 | ET16 | ET2 | ET2 | 17 |
| Embodiment 48 | | | | | | | | | |
| Exemplary | HT16 | HT3 | HT19 | A4 | FF1 | EM16 | ET26 | ET3 | 13 |
| Embodiment 49 | | | | | | | | | |
| Exemplary | HT16 | HT2 | HT15 | B6 | GG3 | EM16 | ET13 | ET2 | 13 |
| Embodiment 50 | | | | | | | | | |
| Exemplary | HT16 | HT2 | HT15 | B18 | HH25 | A4 | ET13 | ET2 | 14 |
| Embodiment 51 | | | | | | | | | |
| Exemplary | HT16 | HT3 | HT19 | B1 | GG21 | EM35 | ET16 | ET15 | 17 |
| Embodiment 52 | | | | | | | | | |
| Exemplary | HT16 | HT3 | HT19 | A7 | DD46 | EM37 | ET16 | ET15 | 16 |
| Embodiment 53 | | | | | | | | | |
| Exemplary | HT16 | HT2 | HT15 | B5 | DD35 | EM30 | ET15 | ET3 | 14 |
| Embodiment 54 | | | | | | | | | |
| Exemplary | HT16 | HT3 | HT19 | B2 | DD31 | EM13 | ET15 | ET15 | 19 |
| Embodiment 55 | | | | | | | | | |
| Exemplary | HT16 | HT2 | HT15 | B3 | DD9 | EM28 | ET2 | ET2 | 18 |
| Embodiment 56 | | | | | | | | | |
| Exemplary | HT16 | HT3 | HT19 | B9 | DD8 | EM28 | ET26 | ET3 | 18 |
| Embodiment 57 | | | | | | | | | |
| Exemplary | HT16 | HT2 | HT15 | EM13 | HH1 | A4 | ET13 | ET2 | 17 |
| Embodiment 58 | | | | | | | | | |
| Exemplary | HT16 | HT3 | HT19 | EM13 | II12 | A11 | ET26 | ET3 | 16 |
| Embodiment 59 | | | | | | | | | |
| Exemplary | HT16 | HT2 | HT15 | EM11 | DD37 | B10 | ET13 | ET2 | 17 |
| Embodiment 60 | | | | | | | | | |
| Exemplary | HT16 | HT2 | HT15 | EM11 | DD46 | B11 | ET13 | ET2 | 17 |
| Embodiment 61 | | | | | | | | | |
| Exemplary | HT16 | HT3 | HT19 | EM13 | B6 | EM29 | ET13 | ET15 | 12 |
| Embodiment 62 | | | | | | | | | |
| Exemplary | HT16 | HT3 | HT19 | EM10 | A1 | EM30 | ET13 | ET15 | 13 |
| Embodiment 63 | | | | | | | | | |

The present disclosure can provide an organic compound with high T1 energy and stability. Thus, such an organic compound can be used to provide an organic light-emitting element with good emission properties and lifetime characteristics.

While the present disclosure has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2021-205221 filed Dec. 17, 2021, which is hereby incorporated by reference herein in its entirety.

What is claimed is:

1. An organic compound represented by the following formula [1]: 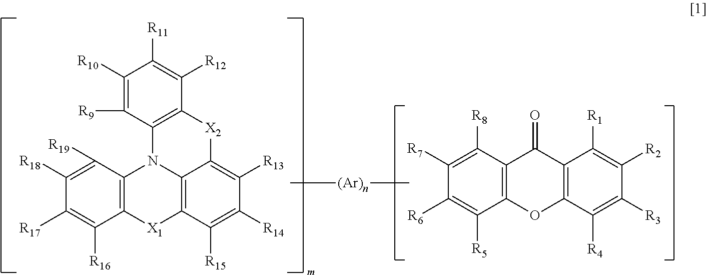
wherein R₁ to R₁₉ are independently selected from the group consisting of a hydrogen atom, a deuterium atom, a halogen atom, a substituted or unsubstituted alkyl group, a substituted or unsubstituted alkoxy group, a substituted or unsubstituted amino group, a substituted or unsubstituted aryloxy group, a substituted or unsubstituted heteroaryloxy group, a substituted or unsubstituted silyl group, a substituted or unsubstituted aromatic hydrocarbon group, a substituted or unsubstituted heterocyclic group, and a cyano group, and R₉ and R₁₉ are optionally bonded together,
in the case that R₉ and R₁₉ are bonded together, the bonding is selected from the group consisting of direct bonding, bonding via an oxygen atom, bonding via a sulfur atom, and bonding via CR₂₀R₂₁, wherein R₂₀ and R₂₁ independently have the same meaning as R₁ to R₁₉,
X₁ and X₂ independently denote an oxygen atom, a sulfur atom, CR₂₀R₂₁, or a single bond,
Ar denotes a substituted or unsubstituted aromatic hydrocarbon group or a substituted or unsubstituted heterocyclic group,
n denotes an integer in the range of 0 to 2, and when n is 0, any one of R₁ to R₈ is directly bonded to any one of R₉ to R₁₉, and
m denotes an integer in the range of 1 to 4.

2. The organic compound according to claim 1, wherein m is 1, n is 1, and any one of R₁ to R₁₉ is bonded to any one of R₂, R₃, R₆, and R₇ via the Ar.

3. The organic compound according to claim 1, wherein m is 1, n is 0, and any one of R₁ to R₁₉ is directly bonded to any one of R₂, R₃, R₆, and R₇.

4. The organic compound according to claim 1, wherein at least one of X₁ and X₂ is CR₂₀R₂₁.

5. The organic compound according to claim 1, wherein at least one of X₁ and X₂ denotes an oxygen atom or a sulfur atom.

6. An organic light-emitting element comprising:
a positive electrode;
a negative electrode; and
one or more organic compound layers between the positive electrode and the negative electrode,
wherein at least one layer of the organic compound layers contains the organic compound according to claim 1.

7. The organic light-emitting element according to claim 6, wherein at least one layer of the organic compound layers is a light-emitting layer.

8. The organic light-emitting element according to claim 7, wherein the light-emitting layer contains a phosphorescent material, and the organic compound constitutes 10% to 99% by mass of the light-emitting layer.

9. The organic light-emitting element according to claim 8, wherein the organic compound constitutes 50% to 99% by mass of the light-emitting layer as a host material.

10. The organic light-emitting element according to claim 8, wherein the organic compound constitutes 10% to 49% by mass of the light-emitting layer as an assist material.

11. The organic light-emitting element according to claim 8, wherein the phosphorescent material is an organometallic complex and has a tricyclic or higher polycyclic fused ring in a ligand thereof.

12. The organic light-emitting element according to claim 11, wherein the organometallic complex is represented by the following formula [2]:
Ir(L)ₘ(L′)ₙ(L″)ᵣ [2]
wherein L, L′, and L″ independently denote a different bidentate ligand,
m is an integer in the range of 1 to 3, and n and r are independently an integer in the range of 0 to 2, provided that m+n+r=3, and
Ir(L)ₘ is represented by any one of the following formulae [Ir-1] to [Ir-11]: 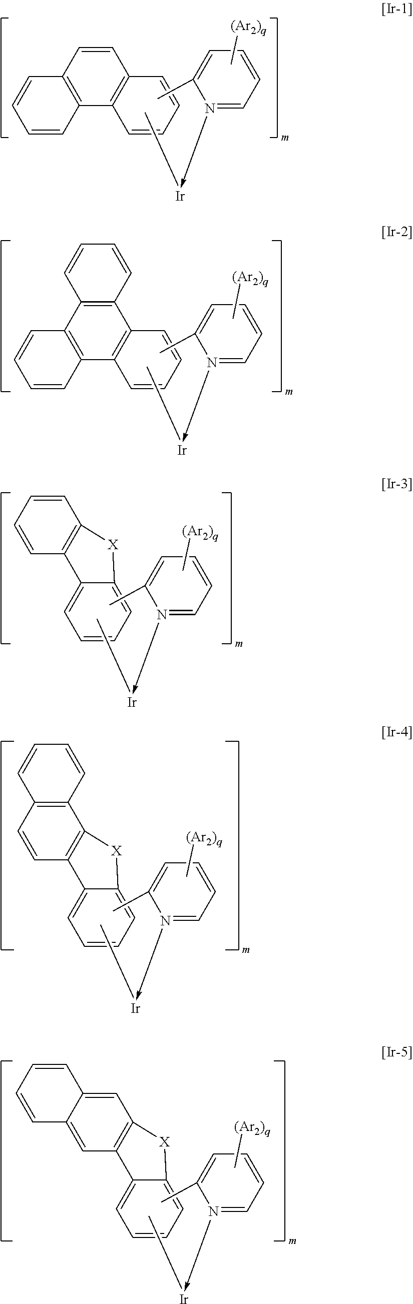 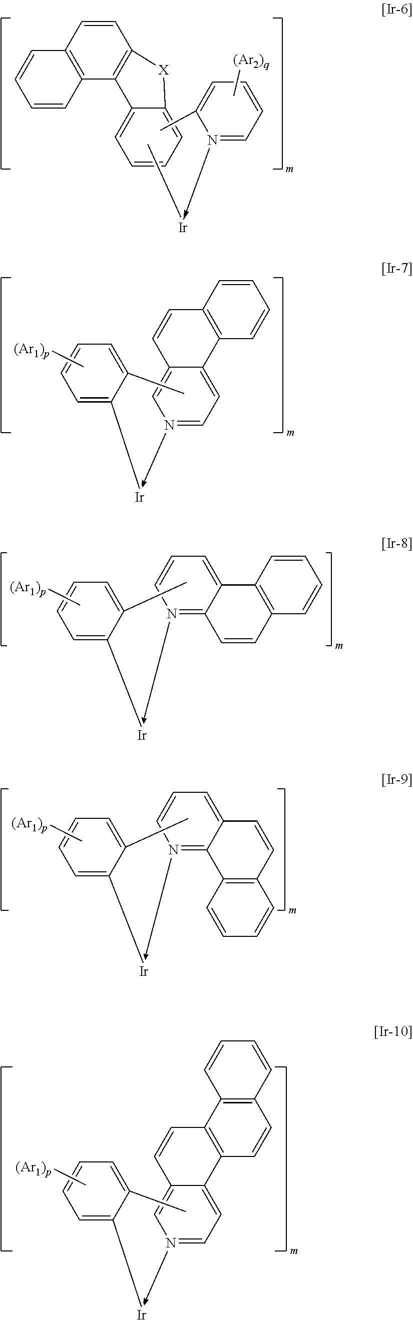 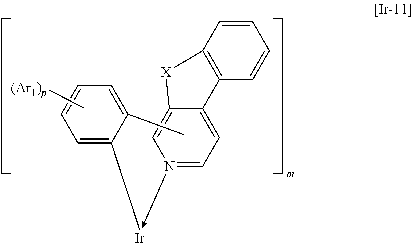
wherein Ar₁ and Ar₂ independently denote a substituted or unsubstituted aryl group or a substituted or unsubstituted heterocyclic group, p and q denote an integer in the range of 0 to 4, and X is selected from the group consisting of an oxygen atom, a sulfur atom, a substituted or unsubstituted carbon atom, and a substituted or unsubstituted nitrogen atom, and
L optionally further having a deuterium atom, a fluorine atom, a substituted or unsubstituted alkyl group, a deuterium-substituted alkyl group, an alkoxy group, a silyl group, a cyano group, a substituted or unsubstituted aryl group, and/or a substituted or unsubstituted heterocyclic group.

13. The organic light-emitting element according to claim 8, wherein the light-emitting layer contains the organic compound, the phosphorescent material, and a third component that is different from the organic compound and the phosphorescent material.

14. The organic light-emitting element according to claim 13, wherein the third component has at least a carbazole skeleton.

15. The organic light-emitting element according to claim 13, wherein the third component has at least an azine ring in a skeleton thereof.

16. The organic light-emitting element according to claim 13, wherein the third component has at least a xanthone skeleton.

17. The organic light-emitting element according to claim 7, wherein the light-emitting layer is a first light-emitting layer, the organic light-emitting element further includes a second light-emitting layer on the first light-emitting layer, and the second light-emitting layer has an emission color different from that of the first light-emitting layer.

18. The organic light-emitting element according to claim 17, wherein the organic light-emitting element emits white light.

19. A display apparatus comprising a plurality of pixels, wherein at least one of the plurality of pixels includes the organic light-emitting element according to claim 6 and a transistor coupled to the organic light-emitting element.

20. An image-forming apparatus comprising:
a photosensitive unit;
a light source configured to expose the photosensitive unit to light; and
a developing unit configured to develop a latent image formed on the photosensitive unit by the light source,
wherein the light source is an exposure light source for an electrophotographic image-forming apparatus, the exposure light source including the organic light-emitting element according to claim 6.

21. A photoelectric conversion apparatus comprising:
an optical unit with a plurality of lenses;
an imaging element configured to receive light passing through the optical unit; and
a display unit configured to display an image taken by the imaging element,
wherein the display unit includes the organic light-emitting element according to claim 6.

22. Electronic equipment comprising:
a display unit including the organic light-emitting element according to claim 6;
a housing in which the display unit is provided; and
a communication unit provided in the housing and configured to communicate with an outside.

23. Alighting apparatus comprising:
a light source including the organic light-emitting element according to claim 6; and
a light-diffusing unit or an optical filter that transmits light emitted by the light source.

24. A moving body comprising:
a lamp including the organic light-emitting element according to claim 6; and
a body to which the lamp is provided.
